(19) United States
(12) Patent Application Publication
(10) Pub. No.: US 2022/0104493 A1
(43) Pub. Date: Apr. 7, 2022

(54) HERBICIDAL COMPOUNDS

(71) Applicant: SYNGENTA CROP PROTECTION AG, Basel (CH)

(72) Inventors: James Nicholas SCUTT, Bracknell, Berkshire (GB); Nigel James WILLETTS, Bracknell, Berkshire (GB); John Stephen DELANEY, Bracknell, Berkshire (GB)

(73) Assignee: SYNGENTA CROP PROTECTION AG, Basel (CH)

(21) Appl. No.: 17/428,433

(22) PCT Filed: Feb. 5, 2020
(86) PCT No.: PCT/EP2020/052814

§ 371 Date: Aug. 4, 2021

(30) Foreign Application Priority Data
Feb. 6, 2019 (GB) 1901617.9

(51) Int. Cl. A01N 43/82; A01P 13/00; A01N 43/76; A01N 43/713; A01N 43/78; A01N 43/80; A01N 43/56

(52) U.S. Cl. CPC A01N 43/82; A01P 13/00; A01N 43/76; A01N 43/56; A01N 43/78; A01N 43/80; A01N 43/713

(57) ABSTRACT

Compounds of the formula (I) wherein the substituents are as defined in claim 1, useful as a pesticides, especially as herbicides.

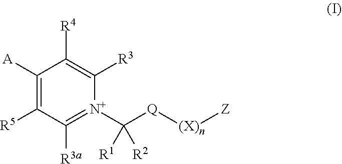

The present invention relates to herbicidally active pyridinium derivatives, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

The present invention is based on the finding that pyridinium derivatives of formula (I) as defined herein, exhibit surprisingly good herbicidal activity. Thus, according to the present invention there is provided the use of a compound of formula (I) or an agronomically acceptable salt or zwitterionic species thereof, as a herbicide:

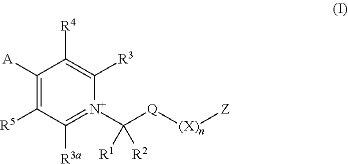

wherein
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O; and

Q is (CR^{1a}R^{2b})ₘ;

m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³, R^{3a}, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different,
and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;
and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;
n is 0 or 1;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2.

Certain compounds of formula (I) are known (or an agronomically acceptable salt or zwitterionic species thereof):

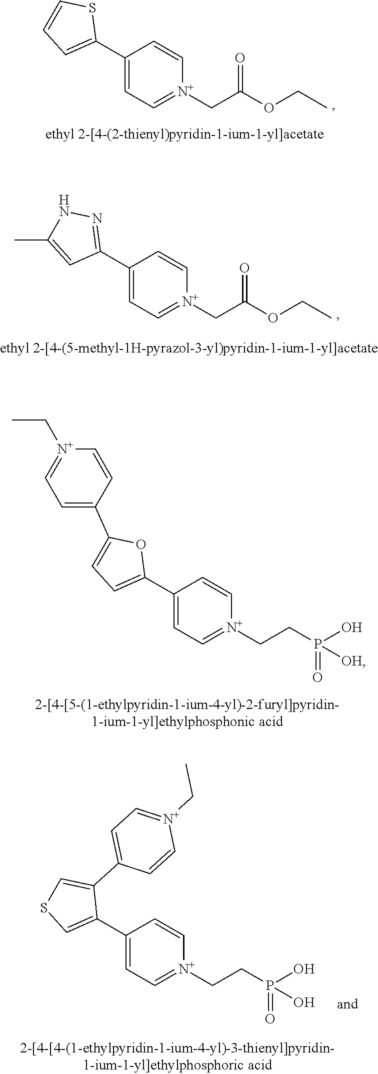

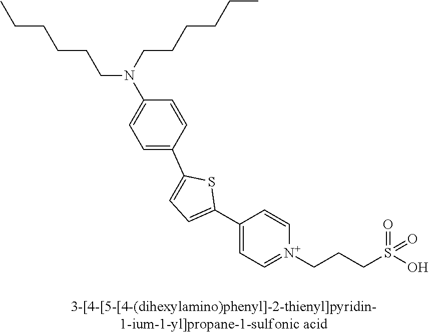 [SMILES: CCCCCCN(CCCCCC)c1ccc(-c2ccc(-c3cc[n+](CCCS(=O)(=O)O)cc3)s2)cc1]

Thus in a second aspect of the invention there is provided a compound of formula (I) wherein:
i) A is not selected from the group consisting of formula A-Ib to A-IIIb below

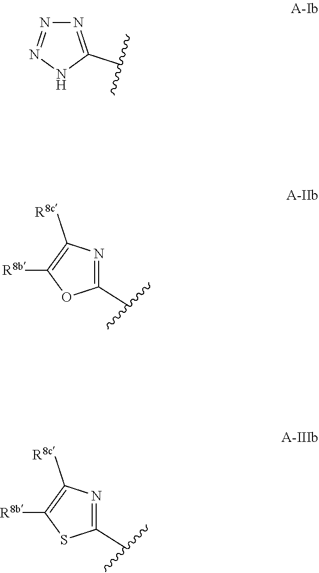

wherein each R^{8b′} is independently selected from the group consisting of phenyl, 4-methoxyphenyl, 4-butoxyphenyl, 4-fluorophenyl and methoxy, and each R^{8c′} is independently hydrogen or methyl;
or
ii) the compound of formula (I) is not selected from the group consisting of ethyl 2-[4-(2-thienyl)pyridin-1-ium-1-yl]acetate, ethyl 2-[4-(5-methyl-1H-pyrazol-3-yl)pyridin-1-ium-1-yl]acetate, 2-[4-[5-(I-ethylpyridin-1-ium-4-yl)-2-furyl]pyridin-1-ium-1-yl]ethylphosphonic acid, 2-[4-[4-(I-ethylpyridin-1-ium-4-yl)-3-thienyl]pyridin-1-ium-1-yl]ethylphosphonic acid and 3-[4-[5-[4-(dihexylamino)phenyl]-2-thienyl]pyridin-1-ium-1-yl]propane-1-sulfonic acid shown above

According to a third aspect of the invention there is provided an agrochemical composition comprising a herbicidally effective amount of a compound of formula (I) and an agrochemically-acceptable diluent or carrier. Such an agricultural composition may further comprise at least one additional active ingredient.

According to a fourth aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fifth aspect of the invention, there is provided the use of a compound of formula (I) as defined herein for pre-harvest desiccation in crops.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl (Me), ethyl (Et), n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁₋₄alkoxy include, but are not limited to, methoxy, ethoxy, propoxy, iso-propoxy and t-butoxy.

As used herein, the term “C₁-C₆haloalkyl” refers to a C₁-C₆alkyl radical as generally defined above substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₅haloalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₃haloalkyl radical as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical as generally defined above, and Rₐ is a C₁-C₃alkylene radical as generally defined above.

As used herein, the term “C₁-C₃alkoxyC₁-C₃alkoxy-” refers to a radical of the formula R_{b}—O—Rₐ—O— where R_{b} is a C₁-C₃alkyl radical as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical as generally defined above.

As used herein, the term “C₃-C₆alkynyloxy” refers to a radical of the formula —ORₐ where Rₐ is a C₃-C₆alkynyl radical as generally defined above.

As used herein, the term “hydroxyC₁-C₆alkyl” refers to a C₁-C₆alkyl radical as generally defined above substituted by one or more hydroxy groups.

As used herein, the term “C₁-C₆alkylcarbonyl” refers to a radical of the formula —C(O)Rₐ where Rₐ is a C₁-C₆alkyl radical as generally defined above.

As used herein, the term “C₁-C₆alkoxycarbonyl” refers to a radical of the formula —C(O)ORₐ where Rₐ is a C₁-C₆alkyl radical as generally defined above.

As used herein, the term “aminocarbonyl” refers to a radical of the formula —C(O)NH₂.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical as generally defined above substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, the term “N—C₃-C₆cycloalkylamino” refers to a radical of the formula —NHRₐ where Rₐ is a C₃-C₆cycloalkyl radical as generally defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 4- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

For example a compound of formula (I) wherein Z comprises an acidic proton, may exist as a zwitterion, a compound of formula (I-I), or as an agronomically acceptable salt, a compound of formula (I-II) as shown below:

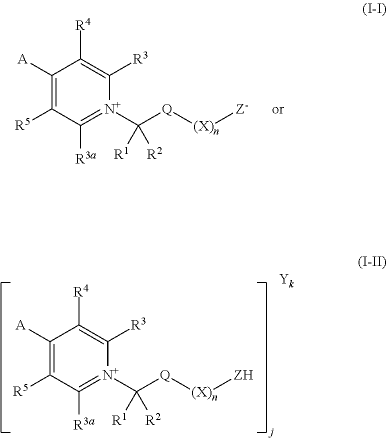

wherein, Y represents an agronomically acceptable anion and j and k represent integers that may be selected from 1, 2 or 3, dependent upon the charge of the respective anion Y.

A compound of formula (I) may also exist as an agronomically acceptable salt of a zwitterion, a compound of formula (I-Ill) as shown below:

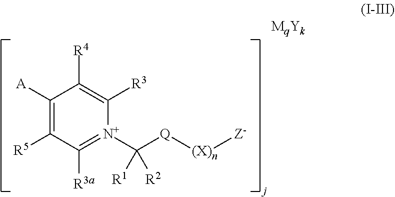

wherein, Y represents an agronomically acceptable anion, M represents an agronomically acceptable cation (in addition to the pyridinium cation) and the integers j, k and q may be selected from 1, 2 or 3, dependent upon the charge of the respective anion Y and respective cation M.

Thus where a compound of formula (I) is drawn in protonated form herein (for example a compound of formula (I-II)), the skilled person would appreciate that it could equally be represented in unprotonated or salt form with one or more relevant counter ions.

In one embodiment of the invention there is provided a compound of formula (I-II) wherein k is 2, j is 1 and Y is selected from the group consisting of halogen, trifluoroacetate and pentafluoropropionate. In this embodiment a nitrogen atom in the ring A may be protonated or a nitrogen atom comprised in R¹, R², Q or X may be protonated. Preferably, in a compound of formula (I-II), k is 2, j is 1 and Y is chloride, wherein a nitrogen atom in the ring comprising A is protonated.

Suitable agronomically acceptable salts of the present invention, represented by an anion Y, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I), wherein Z comprises an acidic proton, can be represented as either (I-I) or (I-II). For compounds of formula (I-II) emphasis is given to salts when Y is chloride, bromide, iodide, hydroxide, bicarbonate, acetate, pentafluoropropionate, triflate, trifluoroacetate, methylsulfate, tosylate and nitrate, wherein j and k are 1. Preferably, Y is chloride, bromide, iodide, hydroxide, bicarbonate, acetate, trifluoroacetate, methylsulfate, tosylate and nitrate, wherein j and k are 1. For compounds of formula (I-II) emphasis is also given to salts when Y is carbonate and sulfate, wherein j is 2 and k is 1, and when Y is phosphate, wherein j is 3 and k is 1.

Where appropriate compounds of formula (I) may also be in the form of (and/or be used as) an N-oxide. Compounds of formula (I) wherein m is 0 and n is 0 may be represented by a compound of formula (I-Ia) as shown below:

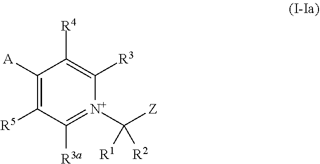

wherein R¹, R², R³, R^{3a}, R⁴, R⁵, A and Z are as defined for compounds of formula (I).

Compounds of formula (I) wherein m is 1 and n is 0 may be represented by a compound of formula (I-Ib) as shown below:

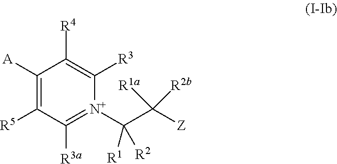

wherein R¹, R², R^{1a}, R^{2b}, R³, R^{3a}, R⁴, R⁵, A and Z are as defined for compounds of formula (I).

Compounds of formula (I) wherein m is 2 and n is 0 may be represented by a compound of formula (I-Ic) as shown below:

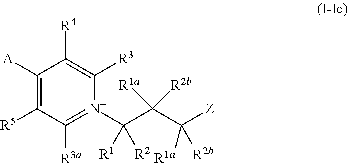

wherein R¹, R², R^{1a}, R^{2b}, R³, R^{3a}, R⁴, R⁵, A and Z are as defined for compounds of formula (I).

Compounds of formula (I) wherein m is 3 and n is 0 may be represented by a compound of formula (I-Id) as shown below:

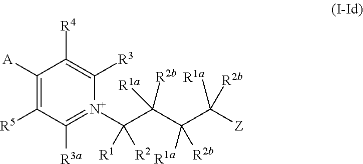

wherein R¹, R², R^{1a}, R^{2b}, R³, R^{3a}, R⁴, R⁵, A and Z are as defined for compounds of formula (I).

The following list provides definitions, including preferred definitions, for substituents n, m, r, A, Q, X, Z, R¹, R², R^{1a}, R², R², R³, R³, R⁴, R⁵, R⁶, R⁷, R^{7b}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ with reference to the compounds of formula (I) according to the invention. For any one of these substituents, any of the definitions given below may be combined with any definition of any other substituent given below or elsewhere in this document.

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵. Preferably, R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵. More preferably, R¹ is selected from the group consisting of hydrogen, halogen, C₁-C₆alkyl, C₁-C₆fluoroalkyl, —OR⁷ and —N(R^{7a})₂. Even more preferably, R¹ is selected from the group consisting of hydrogen, halogen and C₁-C₆alkyl. Even more preferably still, R¹ is hydrogen or C₁-C₆alkyl. Yet even more preferably still, R¹ is hydrogen or methyl. Most preferably R¹ is hydrogen.

R² is selected from the group consisting of hydrogen, halogen, C₁-C₆alkyl and C₁-C₆haloalkyl. Preferably, R² is selected from the group consisting of hydrogen, halogen, C₁-C₆alkyl and C₁-C₆fluoroalkyl. More preferably, R² is hydrogen or C₁-C₆alkyl. Even more preferably, R² is hydrogen or methyl. Most preferably R² is hydrogen.

Wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl. Preferably, when R¹ is selected from the group consisting of —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵, R² is selected from the group consisting of hydrogen and methyl.

Alternatively, R¹ and R² together with the carbon atom to which they are attached form a C₃-C₆cycloalkyl ring or a 3- to 6-membered heterocyclyl, which comprises 1 or 2 heteroatoms individually selected from N and O. Preferably, R¹ and R² together with the carbon atom to which they are attached form a C₃-C₆cycloalkyl ring. More preferably, R¹ and R² together with the carbon atom to which they are attached form a cyclopropyl ring.

In one embodiment R¹ and R² are hydrogen.

In another embodiment R¹ is methyl and R² is hydrogen.

In another embodiment R¹ is methyl and R² is methyl.

Q is (CR^{1a}R^{2b})ₘ.
m is 0, 1, 2 or 3. Preferably, m is 0, 1 or 2. More preferably, m is 0 or 1. Most preferably, m is 0.

Each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵. Preferably, each R^{1a} and R^{2b} are independently selected from the group consisting of hydrogen, halogen, C₁-C₆alkyl, C₁-C₆fluoroalkyl, —OH, —NH₂ and —NHR⁷. More preferably, each Ria and R^{2b} are independently selected from the group consisting of hydrogen, C₁-C₆alkyl, —OH and —NH₂. Even more preferably, each R^{1a} and R^{2b} are independently selected from the group consisting of hydrogen, methyl, —OH and —NH₂. Even more preferably still, each R^{1a} and R^{2b} are independently selected from the group consisting of hydrogen and methyl. Most preferably R^{1a} and R^{2b} are hydrogen.

In another embodiment each R^{1a} and R^{2b} are independently selected from the group consisting of hydrogen and C₁-C₆alkyl.

Alternatively, each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O. Preferably, each R^{1a} and R^{2b} together with the carbon atom to which they are attached form a C₃-C₆cycloalkyl ring. More preferably, each R^{1a} and R^{2b} together with the carbon atom to which they are attached form a cyclopropyl ring.

R³, R^{3a}, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂. Preferably, R³, R^{3a}, R⁴ and R⁵ are independently selected from the group consisting of hydrogen, halogen, cyano, C₁-C₆alkyl, C₁-C₆fluoroalkyl, C₁-C₆fluoroalkoxy, C₁-C₆alkoxy, C₃-C₆cycloalkyl and —N(R⁶)₂. More preferably, R³, R^{3a}, R⁴ and R⁵ are independently selected from the group consisting of hydrogen, halogen, cyano, C₁-C₆alkyl and C₁-C₆fluoroalkyl. Even more preferably, R³, R^{3a}, R⁴ and R⁵ are independently selected from the group consisting of hydrogen, chloro, fluoro, bromo, cyano, methyl and trifluoromethyl. Even more preferably still, R³, R^{3a}, R⁴ and R⁵ are independently selected from the group consisting of hydrogen, chloro, fluoro, bromo and methyl. Yet even more preferably still, R³, R^{3a}, R⁴ and R⁵ are independently selected from the group consisting of hydrogen, chloro and fluoro. Most preferably, R³, R^{3a}, R⁴ and R⁵ are hydrogen.

In one embodiment R³ and R^{3a} are hydrogen, and R⁴ and R⁵ are independently selected from the group consisting of hydrogen, bromo, chloro, fluoro and —S(O)₂Me (preferably, hydrogen, chloro and fluoro).

Each R⁶ is independently selected from hydrogen and C₁-C₆alkyl. Preferably, each R⁶ is independently selected from hydrogen and methyl.

Each R⁷ is independently selected from the group consisting of C₁-C₆alkyl, —S(O)₂R¹⁵, —C(O)R¹⁵, —C(O)OR¹⁵ and —C(O)NR¹⁶R¹⁷. Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl. Most preferably, each R⁷ is methyl.

Each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a}. Preferably, each R^{7a} is independently —C(O)R¹⁵ or —C(O)NR¹⁶R¹⁷.

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different. Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

Alternatively, R^{7b} and R^{7c} together with the nitrogen atom to which they are attached form a 4- to 6-membered heterocyclyl ring which optionally comprises one additional heteroatom individually selected from N, O and S. Preferably, R^{7b} and R^{7c} together with the nitrogen atom to which they are attached form a 5- to 6-membered heterocyclyl ring which optionally comprises one additional heteroatom individually selected from N and O. More preferably, R^{7b} and R^{7c} together with the nitrogen atom to which they are attached form an pyrrolidyl, oxazolidinyl, imidazolidinyl, piperidyl, piperazinyl or morpholinyl group.

A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different.

Preferably, A is a heteroaryl selected from the group consisting of 1,2,4-oxadiazol-5-yl, thiadiazol-5-yl, 1,2,4-thiadiazol-5-yl, thiadiazol-4-yl, 1,2,4-thiadiazol-3-yl, 1,2,5-thiadiazol-3-yl, 1,3,4-thiadiazol-2-yl, 1,3,4-oxadiazol-2-yl, 1,2,4-oxadiazol-3-yl, 1,2,5-oxadiazol-3-yl, 1,2,4-triazol-3-yl, 1,2,4-triazol-5-yl, triazol-4-yl, triazol-5-yl, 2-methyltetrazol-5-yl, 1-methyltetrazol-5-yl, thiazol-2-yl, thiazol-4-yl, isothiazol-5-yl, isothiazol-4-yl, isothiazol-3-yl, oxazol-2-yl, oxazol-4-yl, isoxazol-3-yl, isoxazol-5-yl, imidazol-5-yl, imidazol-2-yl, 3-furyl, 2-furyl, 3-thienyl, pyrazol-5-yl, pyrazol-3-yl and 2-thienyl wherein the heteroaryl may, where feasible, be optionally substituted by 1, 2 or 3 R⁸ substituents, which may be the same or different.

More preferably, A is selected from the group consisting of formula A-I to A-XXXV below

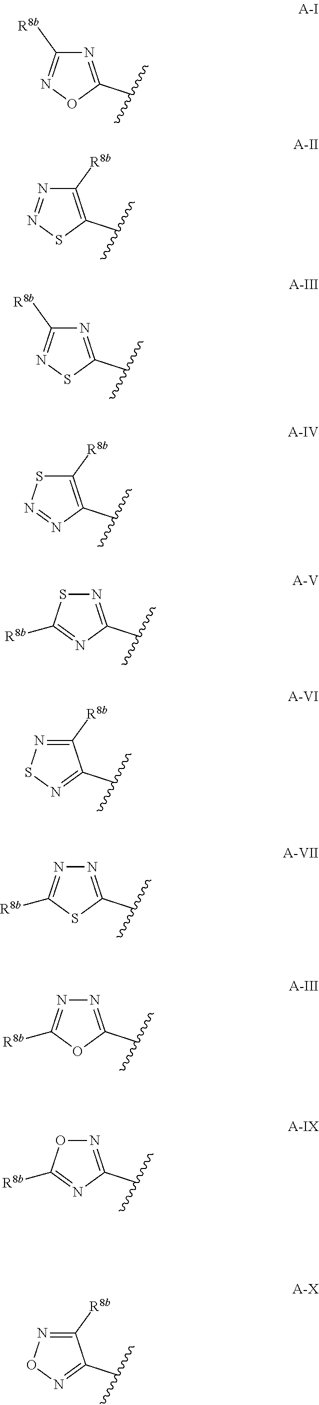

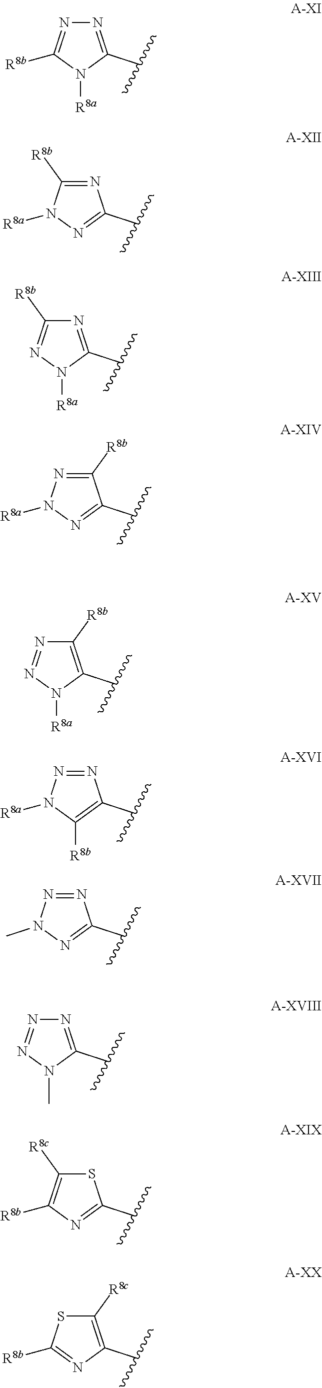

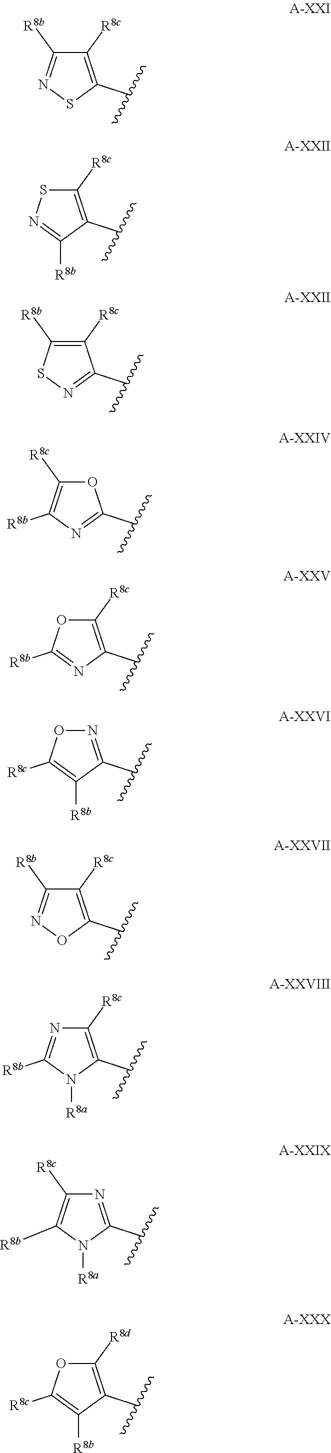

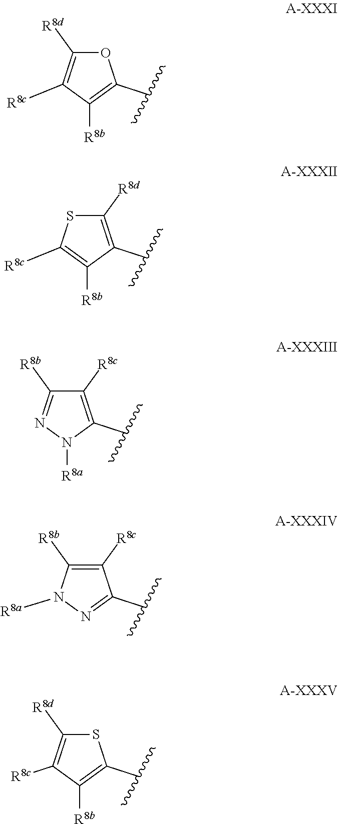

wherein the jagged line defines the point of attachment to a compound of formula (I), and R^{8a}, R^{8b}, R^{8c}, R^{8d}, R¹⁰, R¹⁵, R¹⁶, R¹⁷ and r are as defined herein. R^{8a}, R^{8b}, R^{8c}, R^{8d} are examples of R⁸ wherein the subscript letter a, b, c and d are used to denote positions within individual heterocycles (A-I to A-XXXV).

Even more preferably, A is selected from the group consisting of formula A-I to A-XXXII below

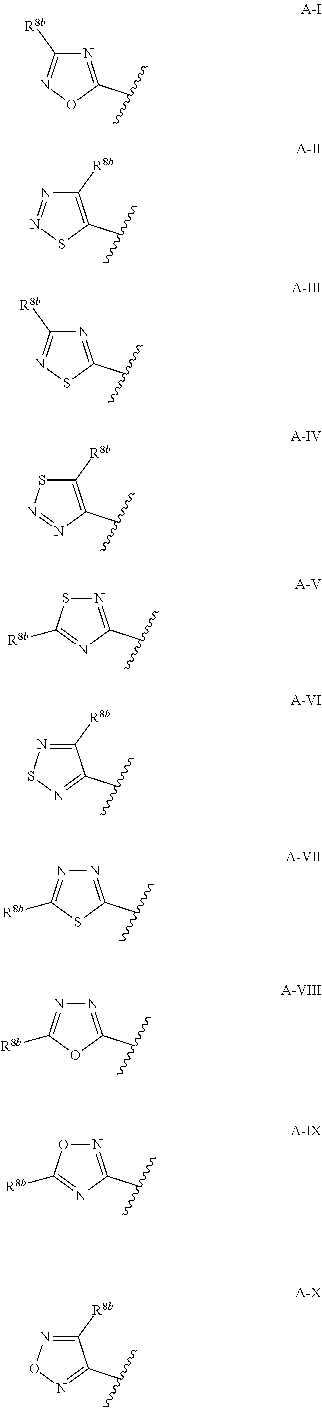

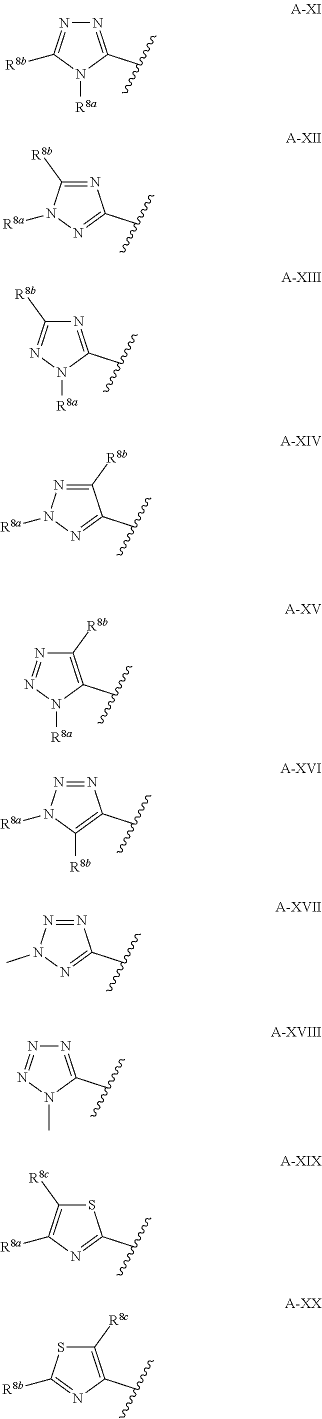

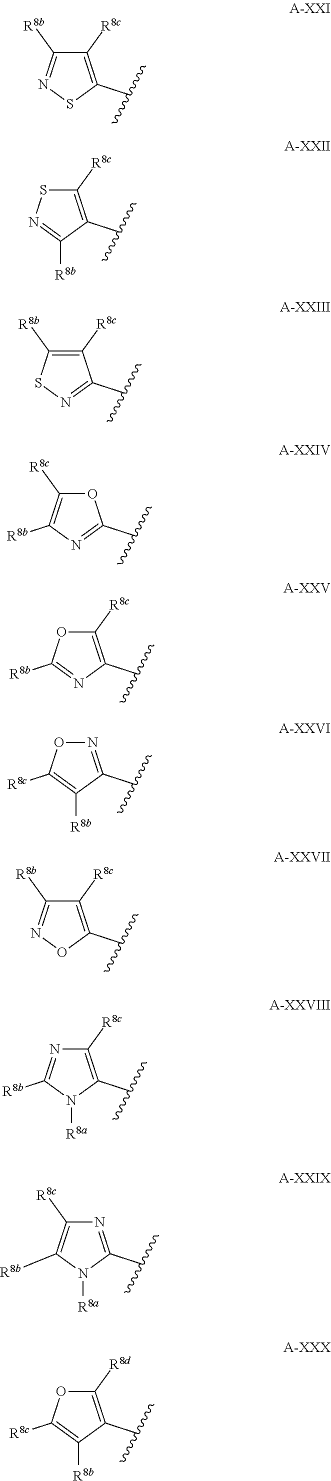

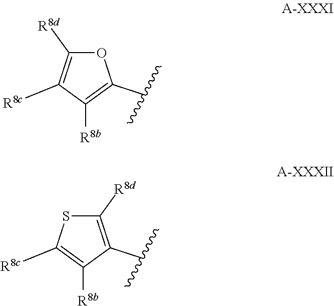

wherein the jagged line defines the point of attachment to a compound of formula (I), and R^{8a}, R^{8b}, R^{8c}, R^{8d}, R¹⁰, R¹¹, R¹⁶, R¹⁷ and r are as defined herein.

Even more preferably still, A is selected from the group consisting of formula A-1 to A-X, A-XVII, A-XVIII, A-XIX, A-XXIII, A-XXIV and AXXVII below

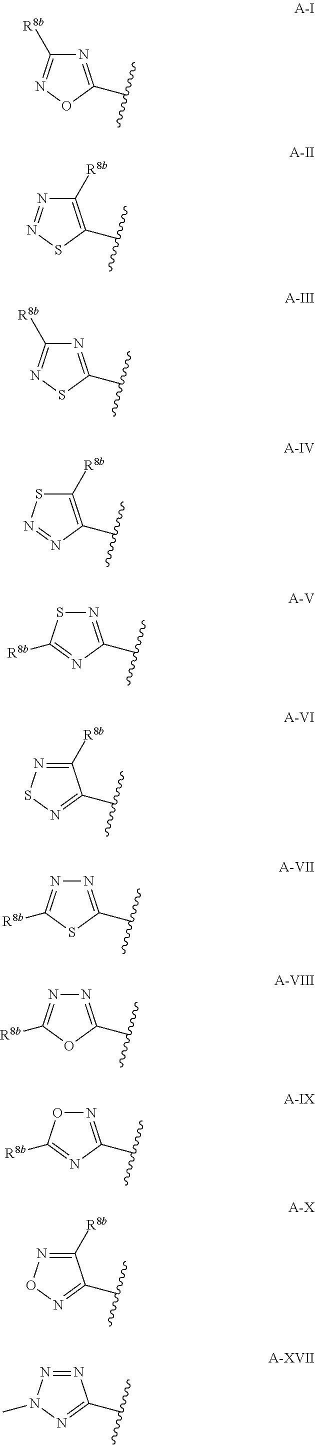

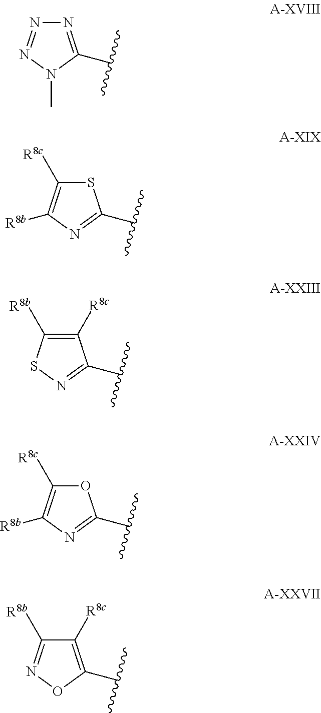

wherein the jagged line defines the point of attachment to a compound of formula (I), and
R^{8a}, R^{8b}, R^{8c}, R^{8d}, R¹⁰, R¹⁵, R¹⁶, R¹⁷ and r are as defined herein.

Yet even more preferably still, A is selected from the group consisting of formula A-I to A-III below

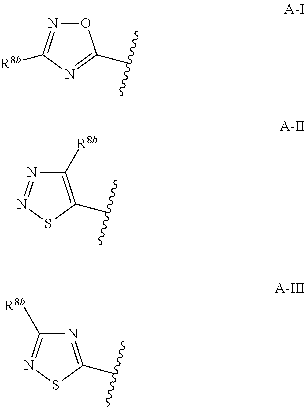

wherein the jagged line defines the point of attachment to a compound of formula (I), and each R^{8b} and R¹⁶ and R¹⁷ are as defined herein.

Further more preferably still, A is selected from the group consisting of formula A-Ia to A-Xa below

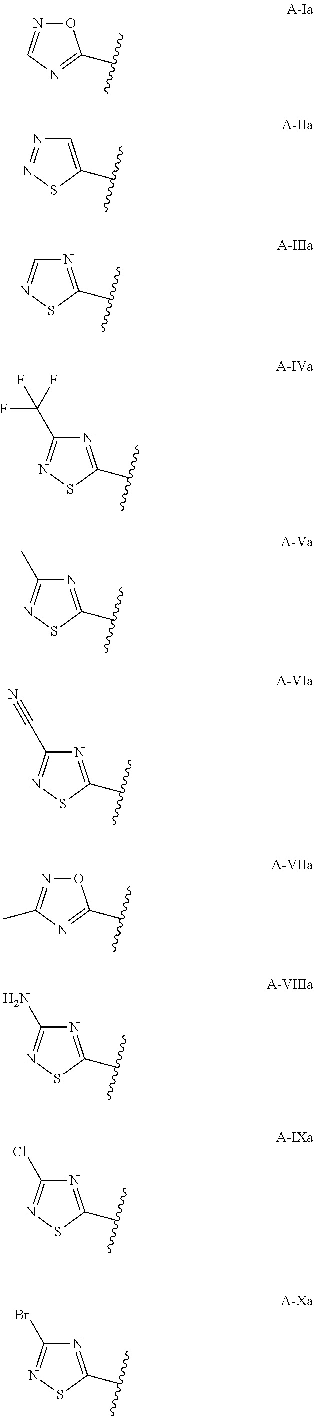

wherein the jagged line defines the point of attachment to a compound of formula (I).

In one embodiment, A is selected from the group consisting of formula A-Ia to A-XXXXVIIa below

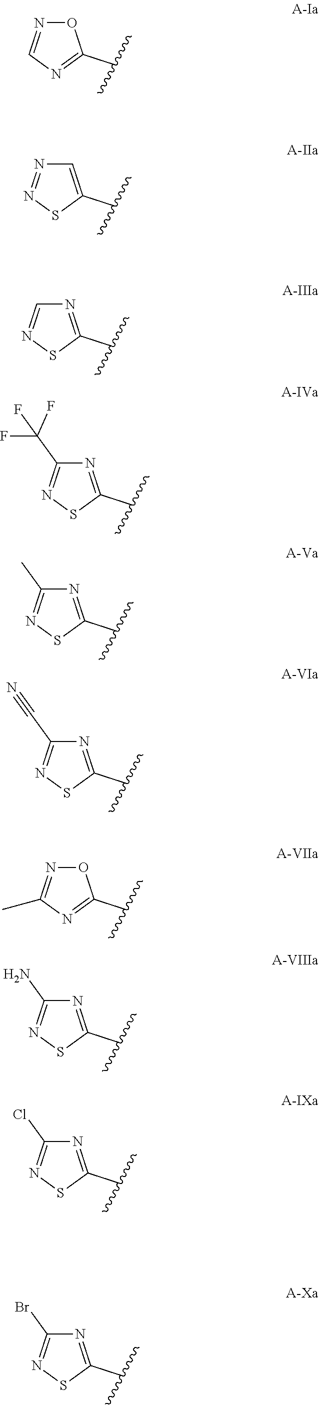

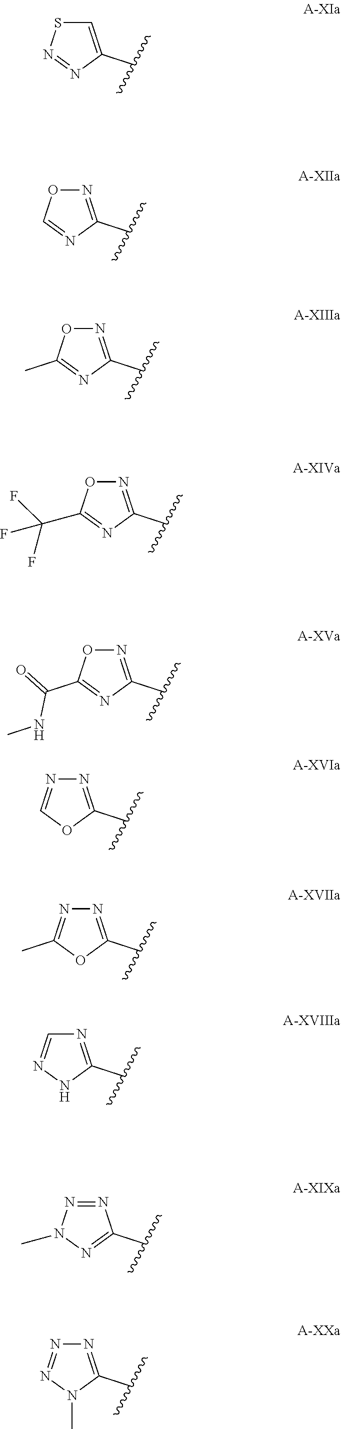

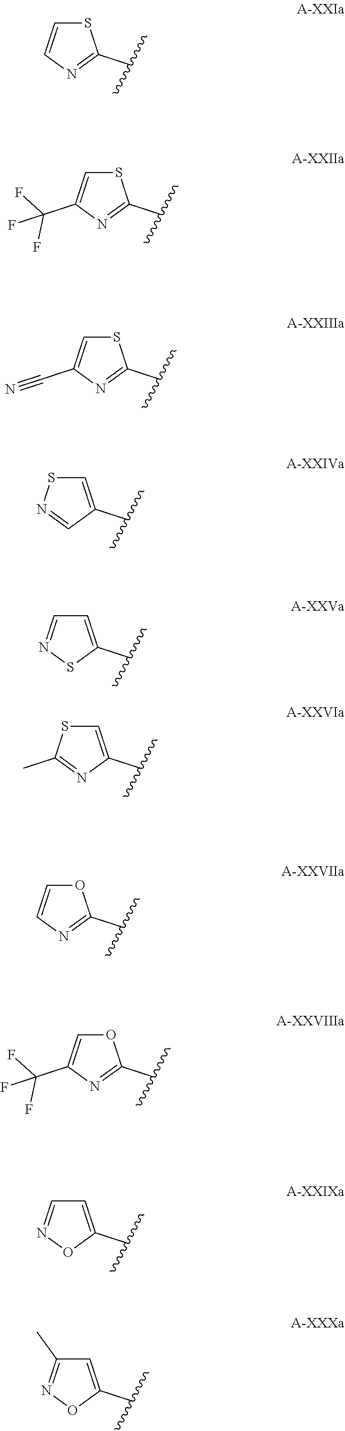

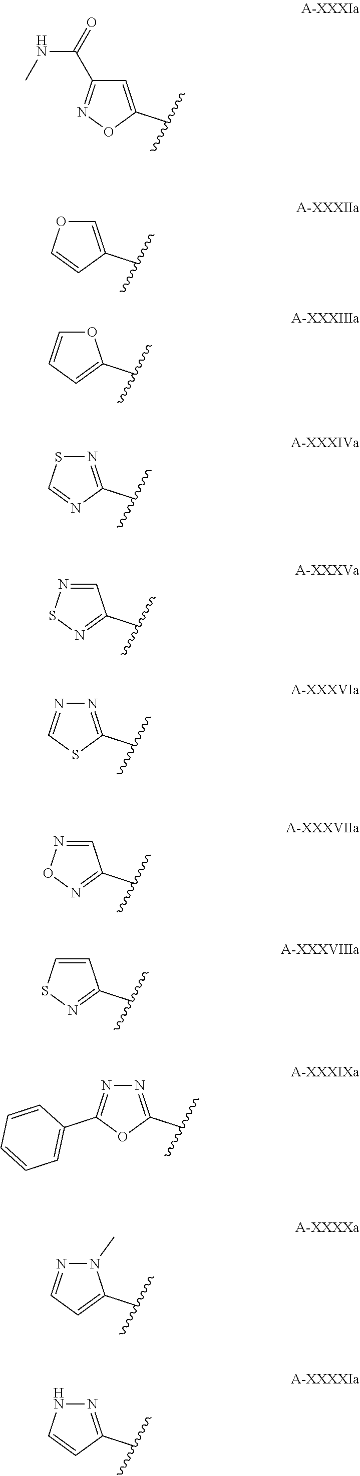

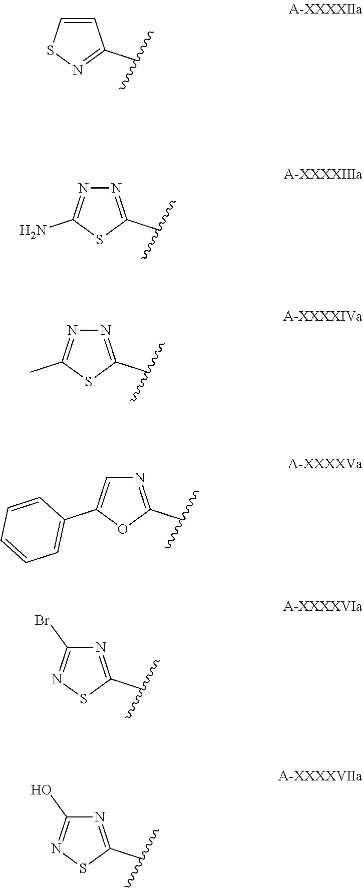

wherein the jagged line defines the point of attachment to a compound of formula (I).

When A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different.

Preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl and a 5- or 6-membered heteroaryl, which comprises 1, 2, 3 or 4 heteroatoms individually selected from N, O and S, and wherein said phenyl or heteroaryl are optionally substituted by 1 or 2 R⁹ substituents, which may be the same or different.

More preferably, when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₁-C₃alkoxyC₁-C₃alkyl-, hydroxyC₁-C₆alkyl-, C₁-C₃alkoxyC₁-C₃alkoxy- and C₁-C₆haloalkoxy.

Even more preferably, when A is substituted on one or more ring carbon atoms, each R⁸ is independently selected from the group consisting of halogen, nitro, cyano, —NH₂, —OH, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl.

Even more preferably still, when A is substituted on one or more ring carbon atoms, each R⁸ is independently selected from the group consisting of halogen, cyano, —NH₂, —OH, —C(O)NR¹⁶R¹⁷, C₁-C₆alkyl and C₁-C₆haloalkyl.

Yet even more preferably still, when A is substituted on one or more ring carbon atoms, each R⁸ is independently selected from the group consisting of halogen, cyano, —NH₂, —C(O)NR¹⁶R¹⁷, C₁-C₆alkyl and C₁-C₆haloalkyl.

Further more preferably still, each R⁸ is independently selected from the group consisting of bromo, chloro, fluoro, cyano, —NH₂, —C(O)NH₂, —C(O)NHMe, —C(O)N(Me)₂, methyl and trifluoromethyl.

Most preferably, when A is substituted on one or more ring carbon atoms, each R⁸ is independently selected from the group consisting of bromo, —NH₂, —C(O)NHMe, methyl and trifluoromethyl.

In one embodiment, when A is substituted on one or more ring carbon atoms, each R⁸ is independently selected from the group consisting of bromo, cyano, —NH₂, —C(O)NHMe, methyl, trifluoromethyl and phenyl (preferably, each R⁸ is independently selected from the group consisting of —C(O)NHMe, methyl and trifluoromethyl).

When A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy. Preferably, R⁸ is selected from the group consisting of —OR⁷, C₁-C₆alkyl and C₁-C₆haloalkyl. More preferably, each R⁸ is C₁-C₆alky or C₁-C₆haloalkyl. Even more preferably still, R⁸ is C₁-C₆alky. Most preferably R⁸ is methyl.

When A is selected from the group consisting of formula A-1 to A-XXXV, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, bromo, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, bromo, —NH₂, —C(O)NHMe, methyl and trifluoromethyl. Even more preferably still, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-1 to A-XXXII, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, bromo, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, bromo, —NH₂, —C(O)NHMe, methyl and trifluoromethyl. Even more preferably still, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-1 to A-X, A-XVII, A-XVIII, A-XIX, A-XXIII, A-XXIV and AXXVII, R^{8a} (substituted on a ring nitrogen atom) is selected from the group consisting of hydrogen, C₁-C₆alkyl and C₁-C₆haloalkyl, and each R^{8b}, R^{8c} and R^{8d} (substituted on a ring carbon atom) are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl. Preferably, R^{8a} is hydrogen or C₁-C₆alkyl and each R^{8b}, R^{8c} and R^{8d} are independently selected from the group consisting of hydrogen, halogen, cyano, —NH₂, —C(O)NR¹⁶R¹⁷, C₁-C₆alkyl and C₁-C₆haloalkyl. More preferably, R^{8a} is hydrogen or methyl and each R^{8b}, R^{8c} and R^{8d} are independently selected from the group consisting of hydrogen, bromo, chloro, fluoro, cyano, —NH₂, —C(O)NH₂, —C(O)NHMe, —C(O)N(Me)₂, methyl and trifluoromethyl. Even more preferably, R^{8a} is hydrogen or methyl and each R^{8b}, R^{8c} and R^{8d} are independently selected from the group consisting of hydrogen, bromo, —NH₂, —C(O)NHMe, methyl and trifluoromethyl. Even more preferably still, R^{8a} is hydrogen or methyl and each R^{8b}, R^{8c} and R^{8d} are independently selected from the group consisting of hydrogen, —C(O)NHMe, methyl and trifluoromethyl.

When A is selected from the group consisting of formula A-1 to A-III, each R^{8b} (substituted on a ring carbon atom) is independently selected from the group consisting of hydrogen, halogen, cyano, —NH₂, —C(O)NR¹⁶R¹⁷, C₁-C₆alkyl and C₁-C₆haloalkyl. Preferably, each R^{8b} is independently selected from the group consisting of hydrogen, bromo, chloro, fluoro, cyano, —NH₂, —C(O)NH₂, —C(O)NHMe, —C(O)N(Me)₂, methyl and trifluoromethyl. More preferably, each R^{8b} is independently selected from the group consisting of hydrogen, bromo, —NH₂, —C(O)NHMe, methyl and trifluoromethyl. Even more preferably, each R^{8b} is independently selected from the group consisting of hydrogen, —C(O)NHMe, methyl and trifluoromethyl.

Each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy. Preferably, each R⁹ is independently selected from the group consisting of halogen, cyano, —N(R⁶)₂, C₁-C₄alkyl, C₁-C₄alkoxy, C₁-C₄haloalkyl and C₁-C₄haloalkoxy. More preferably, each R⁹ is independently selected from the group consisting of halogen, C₁-C₄alkyl, C₁-C₄alkoxy and C₁-C₄haloalkyl. Even more preferably, each R⁹ is independently selected from the group consisting of halogen and C₁-C₄alkyl.

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 substituents, which may be the same or different, selected from R⁹, and wherein the aforementioned CR¹R², Q and Z moieties may be attached at any position of said cycloalkyl, phenyl, heteroaryl or heterocyclyl moieties.

Preferably, X is selected from the group consisting of phenyl and a 4- to 6-membered heterocyclyl, which comprises 1 or 2 heteroatoms individually selected from N and O, and wherein said phenyl or heterocyclyl moieties are optionally substituted by 1 or 2 substituents, which may be the same or different, selected from R⁹, and wherein the aforementioned CR¹R², Q and Z moieties may be attached at any position of said phenyl or heterocyclyl moieties.

More preferably, X is a 4- to 6-membered heterocyclyl, which comprises 1 or 2 heteroatoms individually selected from N and O, and wherein said heterocyclyl moieties is optionally substituted by 1 or 2 substituents, which may be the same or different, selected from R⁹, and wherein the aforementioned CR¹R², Q and Z moieties may be attached at any position of said heterocyclyl moiety.

In one embodiment, X is a 5-membered heterocyclyl, which comprises 1 heteroatom, wherein said heteroatom is N, and wherein the aforementioned CR¹R², Q and Z moieties may be attached at any position of said heterocyclyl moiety. Preferably, X is a 5-membered heterocyclyl, which comprises 1 heteroatom, wherein said heteroatom is N, and wherein the aforementioned CR¹R² and Q moieties are attached adjacent to the N atom and the Z moiety is attached to the N atom.

In another embodiment, X is phenyl optionally substituted by 1 or 2 substituents, which may be the same or different, selected from R⁹, and wherein the aforementioned CR¹R², Q and Z moieties may be attached at any position of said phenyl moiety. Preferably, X is phenyl and the aforementioned CR¹R² and Q moieties are attached in a position para to the Z moiety.

n is 0 or 1. Preferably, n is 0.

Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole.

Preferably, Z is selected from the group consisting of —C(O)OR¹⁰, —C(O)NHOR¹¹, —OC(O)NHOR¹¹, —NR⁶C(O)NHOR¹¹, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHC(O)R¹⁸, —S(O)₂NHS(O)₂R¹², —OS(O)₂NHS(O)₂R¹², —OS(O)₂NHC(O)R¹⁸, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰) and —NR⁶P(O)(R¹³)(OR¹⁰).

More preferably, Z is selected from the group consisting of —C(O)OR¹⁰, —C(O)NHOR¹¹, —C(O)NHS(O)₂R¹², —S(O)₂OR¹⁰, —OS(O)₂OR¹⁰, —NR⁶S(O)₂OR¹⁰, —NHS(O)₂R¹⁴, —S(O)OR¹⁰ and —P(O)(R¹³)(OR¹⁰).

Even more preferably Z is selected from the group consisting of —C(O)OR¹⁰, —C(O)NHS(O)₂R¹², —S(O)₂OR¹⁰, —OS(O)₂OR¹⁰ and —P(O)(R¹³)(OR¹⁰).

Even more preferably still Z is selected from the group consisting of —C(O)OH, —C(O)OCH₃, —C(O)OCH₂CH₃, —C(O)OCH(CH₃)₂, —C(O)OC(CH₃)₃, —C(O)OCH₂C₆H₅, —C(O)OC₆H₅, —C(O)NHS(O)₂CH₃, —S(O)₂OH, —OS(O)₂OH, —P(O)(OH)(OH), —P(O)(OH)(OCH₂CH₃), —P(O)(OH)(OCH₃), —P(O)(OCH₃)(OCH₃), —P(O)(OCH₂CH₃)(OCH₂CH₃), —P(O)(CH₃)(OH) and —P(O)(CH₃)(OCH₂CH₃).

Yet even more preferably still, Z is selected from the group consisting of —C(O)OH, —C(O)OCH₃, —C(O)OCH₂CH₃, —C(O)OC(CH₃)₃, —C(O)NHS(O)₂CH₃, —S(O)₂OH, —OS(O)₂OH, —P(O)(OH)(OH), —P(O)(OH)(OCH₂CH₃), —P(O)(OH)(OCH₃), —P(O)(OCH₃)(OCH₃), —P(O)(OCH₂CH₃)(OCH₂CH₃), —P(O)(CH₃)(OH) and —P(O)(CH₃)(OCH₂CH₃).

Further more preferably still, Z is selected from the group consisting of —C(O)OH, —C(O)NHS(O)₂CH₃, —S(O)₂OH, —OS(O)₂OH, —P(O)(OH)(OH), —P(O)(OH)(OCH₂CH₃), —P(O)(OH)(OCH₃) and —P(O)(CH₃)(OH).

Most preferably Z is —C(O)OH or —S(O)₂OH.

In one embodiment Z is selected from the group consisting of —C(O)OR¹⁰, —C(O)NHS(O)₂R¹², —S(O)₂OR¹⁰, and —P(O)(R¹³)(OR¹⁰) (preferably, —C(O)OH, —C(O)OCH₃, —C(O)OCH₂CH₃, —C(O)NHS(O)₂CH₃, —S(O)₂OH, —P(O)(OH)(OH), —P(O)(OH)(OCH₂CH₃), —P(O)(CH₃)(OH) and —P(O)(CH₃)(OCH₂CH₃)).

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different. Preferably, R¹⁰ is selected from the group consisting of hydrogen, C₁-C₆alkyl, phenyl and benzyl. More preferably, R¹⁰ is selected from the group consisting of hydrogen and C₁-C₆alkyl. Even more preferably, R¹⁰ is selected from the group consisting of hydrogen, methyl, ethyl and tert-butyl. Most preferably, R¹⁰ is hydrogen.

R¹¹ is selected from the group consisting of hydrogen, C₁-C₆alkyl and phenyl, and wherein said phenyl is optionally substituted by 1, 2 or 3 R⁹ substituents, which may be the same or different. Preferably, R¹¹ is selected from the group consisting of hydrogen, C₁-C₆alkyl and phenyl. More preferably, R¹¹ is selected from the group consisting of hydrogen and C₁-C₆alkyl. Even more preferably, R¹¹ is C₁-C₆alkyl. Most preferably, R¹¹ is methyl.

R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different. Preferably, R¹² is selected from the group consisting of C₁-C₆alkyl, C₁-C₆haloalkyl, C₁-C₆alkoxy, —OH, —N(R⁶)₂ and phenyl. More preferably, R¹² is selected from the group consisting of C₁-C₆alkyl, C₁-C₆haloalkyl and —N(R⁶)₂. Even more preferably, R¹² is selected from the group consisting of methyl, —N(Me)₂ and trifluoromethyl. Most preferably, R¹² is methyl.

R¹³ is selected from the group consisting of —OH, C₁-C₆alkyl, C₁-C₆alkoxy and phenyl. Preferably R¹³ is selected from the group consisting of —OH, C₁-C₆alkyl and C₁-C₆alkoxy. More preferably, R¹³ is selected from the group consisting of —OH and C₁-C₆alkoxy. Even more preferably, R¹³ is selected from the group consisting of —OH, methoxy and ethoxy. Most preferably, R¹³ is —OH.

R¹⁴ is C₁-C₆haloalkyl. Preferably, R¹⁴ is trifluoromethyl.

R¹⁵ is selected from the group consisting of C₁-C₆alkyl and phenyl, and wherein said phenyl is optionally substituted by 1, 2 or 3 R⁹ substituents, which may be the same or different. Preferably, R¹⁵ is selected from the group consisting of C₁-C₆alkyl and phenyl. More preferably, R¹⁵ is C₁-C₆alkyl. Most preferably R¹⁵ is methyl.

R^{15a} is phenyl, wherein said phenyl is optionally substituted by 1, 2 or 3 R⁹ substituents, which may be the same or different. Preferably, R^{15a} is phenyl optionally substituted by 1 R⁹ substituent. More preferably, R^{15a} is phenyl.

R¹⁶ and R¹⁷ are independently selected from the group consisting of hydrogen and C₁-C₆alkyl. Preferably, R¹⁶ and R¹⁷ are independently selected from the group consisting of hydrogen and methyl.

Alternatively, R¹⁶ and R¹⁷ together with the nitrogen atom to which they are attached form a 4- to 6-membered heterocyclyl ring which optionally comprises one additional heteroatom individually selected from N, O and S. Preferably, R¹⁶ and R¹⁷ together with the nitrogen atom to which they are attached form a 5- to 6-membered heterocyclyl ring which optionally comprises one additional heteroatom individually selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different. Preferably, R¹⁸ is selected from the group consisting of hydrogen, C₁-C₆alkyl, C₁-C₆haloalkyl, C₁-C₆alkoxy, —N(R⁶)₂ and phenyl. More preferably, R¹⁸ is selected from the group consisting of hydrogen, C₁-C₆alkyl and C₁-C₆haloalkyl. Further more preferably, R¹⁸ is selected from the group consisting of C₁-C₆alkyl and C₁-C₆haloalkyl. Most preferably, R¹⁸ is methyl or trifluoromethyl.

### r is 0, 1 or 2. Preferably, r is 0 or 2.

In a set of preferred embodiments, in a compound according to formula (I) of the invention,

R¹ is hydrogen or C₁-C₆alkyl;
R² is hydrogen or methyl;
Q is (CR^{1a}R^{2b})ₘ;
m is 0, 1 or 2;
R^{1a} and R^{2b} are independently selected from the group consisting of hydrogen, C₁-C₆alkyl, —OH and —NH₂;
R³, R^{3a}, R⁴ and R⁵ are independently selected from the group consisting of hydrogen, chloro, fluoro, bromo and methyl;
each R⁶ is independently selected from hydrogen and methyl;
each R⁷ is C₁-C₆alkyl;

A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different;

when A is substituted on one or more ring carbon atoms, each R⁸ is independently selected from the group consisting of halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;
and/or
when A is substituted on a ring nitrogen atom, R⁸ is C₁-C₆alky or C₁-C₆haloalkyl; and
n is 0;
Z is selected from the group consisting of —C(O)OR¹⁰, —C(O)NHS(O)₂R¹², —S(O)₂OR¹⁰, —OS(O)₂OR¹⁰ and —P(O)(R¹³)(OR¹⁰);
R¹⁰ is selected from the group consisting of hydrogen, C₁-C₆alkyl, phenyl and benzyl;
R¹² is selected from the group consisting of C₁-C₆alkyl, C₁-C₆haloalkyl and —N(R⁶)₂;
R¹³ is selected from the group consisting of —OH, C₁-C₆alkyl and C₁-C₆alkoxy;
R¹⁵ is C₁-C₆alkyl;
R¹⁶ and R¹⁷ are independently selected from the group consisting of hydrogen and methyl; and
r is 0 or 2.

More preferably,

R¹ is hydrogen or methyl;
R² is hydrogen or methyl;
Q is (CR^{1a}R^{2b})ₘ;
m is 0 or 1;
R^{1a} and R^{2b} are independently selected from the group consisting of hydrogen and methyl;
R³, R^{3a}, R⁴ and R⁵ are independently selected from the group consisting of hydrogen, chloro and fluoro; A is a heteroaryl selected from the group consisting of 1,2,4-oxadiazol-5-yl, thiadiazol-5-yl, 1,2,4-thiadiazol-5-yl, thiadiazol-4-yl, 1,2,4-thiadiazol-3-yl, 1,2,5-thiadiazol-3-yl, 1,3,4-thiadiazol-2-yl, 1,3,4-oxadiazol-2-yl, 1,2,4-oxadiazol-3-yl, 1,2,5-oxadiazol-3-yl, 1,2,4-triazol-3-yl, 1,2,4-triazol-5-yl, triazol-4-yl, triazol-5-yl, 2-methyltetrazol-5-yl, 1-methyltetrazol-5-yl, thiazol-2-yl, thiazol-4-yl, isothiazol-5-yl, isothiazol-4-yl, isothiazol-3-yl, oxazol-2-yl, oxazol-4-yl, isoxazol-3-yl, isoxazol-5-yl, imidazol-5-yl, imidazol-2-yl, 3-furyl, 2-furyl, 3-thienyl, pyrazol-5-yl, pyrazol-3-yl and 2-thienyl wherein the heteroaryl may, where feasible, be optionally substituted by 1, 2 or 3 R⁸ substituents, which may be the same or different;
when A is substituted on one or more ring carbon atoms, each R⁸ is independently selected from the group consisting of halogen, cyano, —NH₂, —C(O)NR¹⁶R¹⁷, C₁-C₆alkyl and C₁-C₆haloalkyl;
and/or
when A is substituted on a ring nitrogen atom, R⁸ is C₁-C₆alkyl; and
n is 0;
Z is selected from the group consisting of —C(O)OH, —C(O)OCH₃, —C(O)OCH₂CH₃, —C(O)OCH(CH₃)₂, —C(O)OC(CH₃)₃, —C(O)OCH₂C₆H₅, —C(O)OC₆H₅, —C(O)NHS(O)₂CH₃, —S(O)₂OH, —OS(O)₂OH, —P(O)(OH)(OH), —P(O)(OH)(OCH₂CH₃), —P(O)(OH)(OCH₃), —P(O)(OCH₃)(OCH₃), —P(O)(OCH₂CH₃)(OCH₂CH₃), —P(O)(CH₃)(OH) and —P(O)(CH₃)(OCH₂CH₃); and
R¹⁶ and R¹⁷ are independently selected from the group consisting of hydrogen and methyl.

In a further set of preferred embodiments, the compound according to formula (I) is selected from a compound of formula (I-a), (I-b), (I-c), (I-d), (I-e) or (I-f),

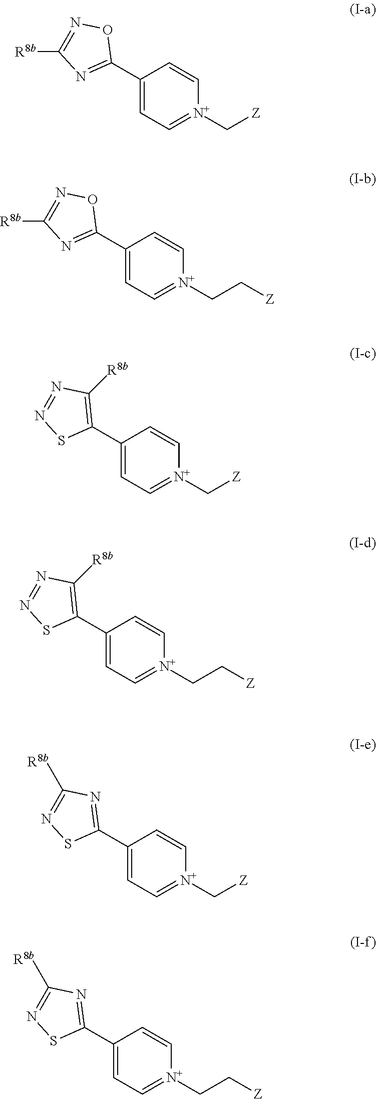

wherein in a compound of formula (I-a), (I-b), (I-c), (I-d), (I-e) and (I-f),
each R^{8b} is independently selected from the group consisting of hydrogen, bromo, chloro, fluoro, cyano, —NH₂, —C(O)NH₂, —C(O)NHMe, —C(O)N(Me)₂, methyl and trifluoromethyl; and
Z is selected from the group consisting of —C(O)OH, —C(O)OCH₃, —C(O)OCH₂CH₃, —C(O)OC(CH₃)₃, —C(O)NHS(O)₂CH₃, —S(O)₂OH, —OS(O)₂OH, —P(O)(OH)(OH), —P(O)(OH)(OCH₂CH₃), —P(O)(OH)(OCH₃), —P(O)(OCH₃)(OCH₃), —P(O)(OCH₂CH₃)(OCH₂CH₃), —P(O)(CH₃)(OH) and —P(O)(CH₃)(OCH₂CH₃).

In a further more preferred set of embodiments, the compound according to formula (I) is selected from a compound of formula (I-aa), (I-bb), (I-cc), (I-dd), (I-ee) or (I-ff),

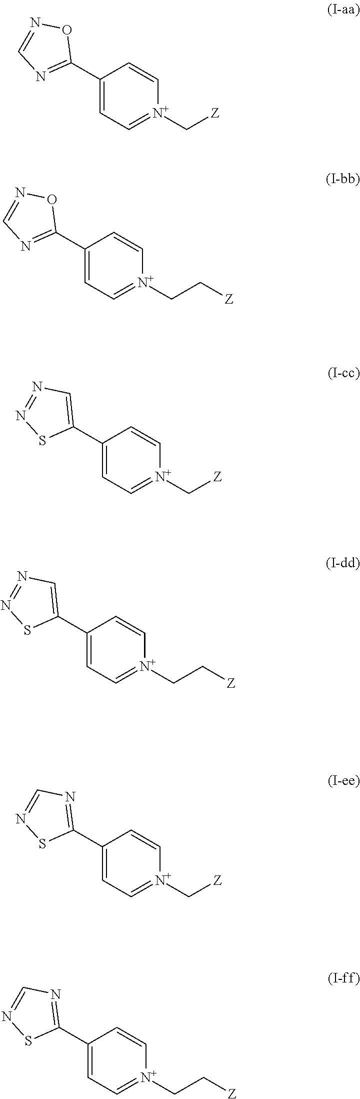

wherein in a compound of formula (I-aa), (I-bb), (I-cc), (I-dd), (I-ee) and (I-ff),

### Z is —C(O)OH or —S(O)₂OH.

In a another set of preferred embodiments, the compound according to formula (I) is selected from a compound of formula (I-h), (I-k) or (I-m),

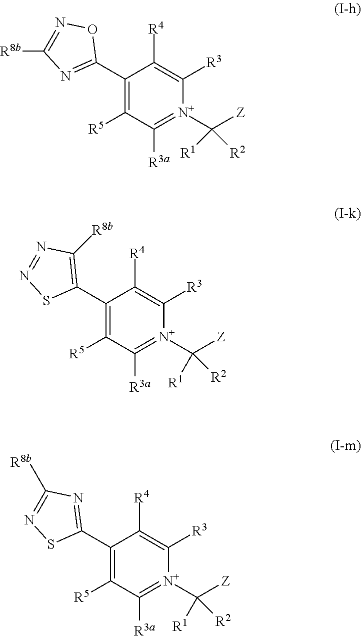

wherein in a compound of formula (I-h), (I-k) or (I-m),
R¹ is hydrogen or methyl;
R² is hydrogen or methyl;
R³, R^{3a}, R⁴ and R⁵ are independently selected from the group consisting of hydrogen, chloro, fluoro, bromo, cyano, methyl and trifluoromethyl;
each R⁶ is independently hydrogen or methyl;
each R^{8b} is independently selected from the group consisting of hydrogen, halogen, cyano, —NH₂, —C(O)NR¹⁶R¹⁷, C₁-C₆alkyl and C₁-C₆haloalkyl;
Z is selected from the group consisting of —C(O)OR¹⁰, —C(O)NHS(O)₂R¹², —S(O)₂OR¹⁰, and —P(O)(R¹³)(OR¹⁰);
R¹⁰ is selected from the group consisting of hydrogen and C₁-C₆alkyl;
R¹² is selected from the group consisting of C₁-C₆alkyl, C₁-C₆haloalkyl and —N(R⁶)₂;
R¹³ is selected from the group consisting of —OH, C₁-C₆alkyl and C₁-C₆alkoxy; and
R¹⁶ and R¹⁷ are independently selected from the group consisting of hydrogen and methyl.

In one set of embodiments, the compound according to formula (I) is selected from a compound A1 to A101 listed in Table A.

It should be understood that compounds of formula (I) may exist/be manufactured in ‘procidal form’, wherein they comprise a group ‘G’. Such compounds are referred to herein as compounds of formula (I-IV).

G is a group which may be removed in a plant by any appropriate mechanism including, but not limited to, metabolism and chemical degradation to give a compound of formula (I-I), (I-II) or (I-III) wherein Z contains an acidic proton, for example see the scheme below:

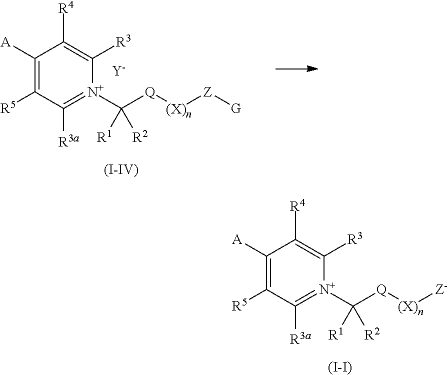

Whilst such G groups may be considered as ‘procidal’, and thus yield active herbicidal compounds once removed, compounds comprising such groups may also exhibit herbicidal activity in their own right. In such cases in a compound of formula (I-IV), Z-G may include but is not limited to, any one of (G1) to (G7) below and E indicates the point of attachment to the remaining part of a compound of formula (I):

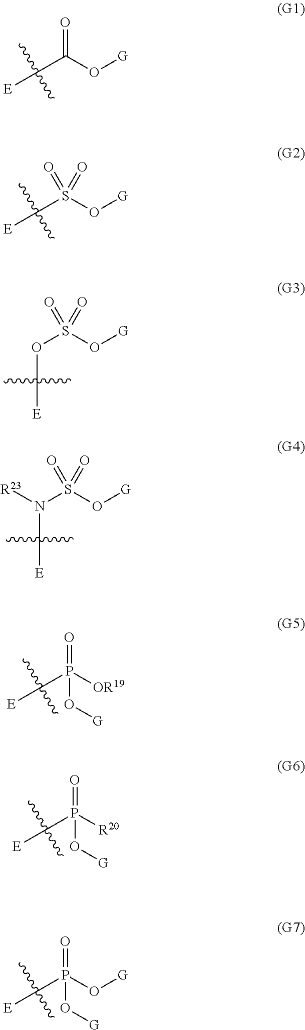

In embodiments where Z-G is (G1) to (G7), G, R¹⁹, R²⁰, R²¹, R²² and R²³ are defined as follows:

G is C₁-C₆alkyl, C₂-C₆alkenyl, C₂-C₆alkynyl, —C(R²¹R²²)OC(O)R¹⁹, phenyl or phenyl-C₁-C₄alkyl-, wherein said phenyl moiety is optionally substituted by 1 to 5 substituents independently selected from halo, cyano, nitro, C₁-C₆alkyl, C₁-C₆haloalkyl or C₁-C₆alkoxy.

R¹⁹ is C₁-C₆alkyl or phenyl,
R²⁰ is hydroxy, C₁-C₆alkyl, C₁-C₆alkoxy or phenyl,
R²¹ is hydrogen or methyl,
R²² is hydrogen or methyl,
R²³ is hydrogen or C₁-C₆alkyl.

The compounds in Tables 1 to 27 below illustrate the compounds of the invention. The skilled person would understand that the compounds of formula (I) may exist as an agronomically acceptable salt, a zwitterion or an agronomically acceptable salt of a zwitterion as described hereinbefore.

TABLE 1

| Com- | | | | | | | |
|---|---|---|---|---|---|---|---|
| pound | | | | | | | |
| number | R³ | R^{3a} | R⁴ | R⁵ | Z | m | Q |
| 1.001 | H | H | H | H | —C(O)OH | 0 | — |
| 1.002 | H | H | H | H | —C(O)OMe | 0 | — |
| 1.003 | H | H | H | H | —C(O)NHOMe | 0 | — |
| 1.004 | H | H | H | H | —OC(O)NHOMe | 0 | — |
| 1.005 | H | H | H | H | —NHC(O)NHOMe | 0 | — |
| 1.006 | H | H | H | H | —NMeC(O)NHOMe | 0 | — |
| 1.007 | H | H | H | H | —C(O)NHS(O)₂Me | 0 | — |
| 1.008 | H | H | H | H | —OC(O)NHS(O)₂Me | 0 | — |
| 1.009 | H | H | H | H | —NHC(O)NHS(O)₂Me | 0 | — |
| 1.010 | H | H | H | H | —NMeC(O)NHS(O)₂Me | 0 | — |
| 1.011 | H | H | H | H | —S(O)₂OH | 0 | — |
| 1.012 | H | H | H | H | —OS(O)₂OH | 0 | — |
| 1.013 | H | H | H | H | —NHS(O)₂OH | 0 | — |
| 1.014 | H | H | H | H | —NMeS(O)₂OH | 0 | — |
| 1.015 | H | H | H | H | —S(O)OH | 0 | — |
| 1.016 | H | H | H | H | —OS(O)OH | 0 | — |
| 1.017 | H | H | H | H | —NHS(O)OH | 0 | — |
| 1.018 | H | H | H | H | —NMeS(O)OH | 0 | — |
| 1.019 | H | H | H | H | —NHS(O)₂CF₃ | 0 | — |
| 1.020 | H | H | H | H | —S(O)₂NHC(O)Me | 0 | — |
| 1.021 | H | H | H | H | —OS(O)₂NHC(O)Me | 0 | — |
| 1.022 | H | H | H | H | —NHS(O)₂NHC(O)Me | 0 | — |
| 1.023 | H | H | H | H | —NMeS(O)₂NHC(O)Me | 0 | — |
| 1.024 | H | H | H | H | —P(O)(OH)(OMe) | 0 | — |
| 1.025 | H | H | H | H | —P(O)(OH)(OH) | 0 | — |
| 1.026 | H | H | H | H | —OP(O)(OH)(OMe) | 0 | — |
| 1.027 | H | H | H | H | —OP(O)(OH)(OH) | 0 | — |
| 1.028 | H | H | H | H | —NHP(O)(OH)(OMe) | 0 | — |
| 1.029 | H | H | H | H | —NHP(O)(OH)(OH) | 0 | — |
| 1.030 | H | H | H | H | —NMeP(O)(OH)(OMe) | 0 | — |
| 1.031 | H | H | H | H | —NMeP(O)(OH)(OH) | 0 | — |
| 1.032 | H | H | H | H | —tetrazole | 0 | — |
| 1.033 | H | H | H | H | —S(O)₂OH | 1 | CH(NH₂) |
| 1.033 | H | H | H | H | —C(O)OH | 1 | CH(NH₂) |
| 1.035 | H | H | H | H | —S(O)₂OH | 2 | CH(OH)CH₂ |
| 1.036 | H | H | H | H | —C(O)OH | 2 | CH(OH)CH₂ |
| 1.037 | H | H | H | H | —S(O)₂OH | 1 | CH(OH) |
| 1.038 | H | H | H | H | —C(O)OH | 1 | CH(OH) |
| 1.039 | H | H | H | H | —C(O)NHCN | 0 | — |
| 1.040 | H | H | H | H | —OC(O)NHCN | 0 | — |
| 1.041 | H | H | H | H | —NHC(O)NHCN | 0 | — |
| 1.042 | H | H | H | H | —NMeC(O)NHCN | 0 | — |
| 1.043 | H | H | H | H | —S(O)₂NHCN | 0 | — |
| 1.044 | H | H | H | H | —OS(O)₂NHCN | 0 | — |
| 1.045 | H | H | H | H | —NHS(O)₂NHCN | 0 | — |
| 1.046 | H | H | H | H | —NMeS(O)₂NHCN | 0 | — |
| 1.047 | H | H | H | H | —S(O)₂NHS(O)₂Me | 0 | — |
| 1.048 | H | H | H | H | —OS(O)₂NHS(O)₂Me | 0 | — |
| 1.049 | H | H | H | H | —NHS(O)₂NHS(O)₂Me | 0 | — |
| 1.050 | H | H | H | H | —NMeS(O)₂NHS(O)₂Me | 0 | — |
| 1.051 | H | H | H | H | —P(O)H(OH) | 0 | — |
| 1.052 | H | H | H | H | —N(OH)C(O)Me | 0 | — |
| 1.053 | H | H | H | H | —ONHC(O)Me | 0 | — |

This table discloses 53 specific compounds (1.001 to 1.053) of the formula (T-1):

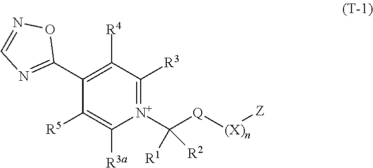

Wherein m, Q, R³, R^{3a}, R⁴, R⁵ and Z are as defined in Table 1, R¹ and R² are hydrogen and n is 0.

This table discloses 49 specific compounds 2.001 to 2.049) of the formula (T-2):

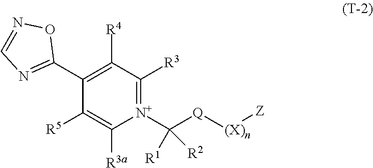

Wherein m, Q, R³, R^{3a}, R⁴, R⁵ and Z are as defined in Table 2, R¹ and R² are hydrogen and n is 0.

TABLE 2

| Com- | | | | | | | |
|---|---|---|---|---|---|---|---|
| pound | | | | | | | |
| number | R³ | R^{3a} | R⁴ | R⁵ | Z | m | Q |
| 2.001 | H | H | H | H | —C(O)OH | 1 | CH₂ |
| 2.002 | H | H | H | H | —C(O)OMe | 1 | CH₂ |
| 2.003 | H | H | H | H | —C(O)NHOMe | 1 | CH₂ |
| 2.004 | H | H | H | H | —OC(O)NHOMe | 1 | CH₂ |
| 2.005 | H | H | H | H | —NHC(O)NHOMe | 1 | CH₂ |
| 2.006 | H | H | H | H | —NMeC(O)NHOMe | 1 | CH₂ |
| 2.007 | H | H | H | H | —C(O)NHS(O)₂Me | 1 | CH₂ |
| 2.008 | H | H | H | H | —OC(O)NHS(O)₂Me | 1 | CH₂ |
| 2.009 | H | H | H | H | —NHC(O)NHS(O)₂Me | 1 | CH₂ |
| 2.010 | H | H | H | H | —NMeC(O)NHS(O)₂Me | 1 | CH₂ |
| 2.011 | H | H | H | H | —S(O)₂OH | 1 | CH₂ |
| 2.012 | H | H | H | H | —OS(O)₂OH | 1 | CH₂ |
| 2.013 | H | H | H | H | —NHS(O)₂OH | 1 | CH₂ |
| 2.014 | H | H | H | H | —NMeS(O)₂OH | 1 | CH₂ |
| 2.015 | H | H | H | H | —S(O)OH | 1 | CH₂ |
| 2.016 | H | H | H | H | —OS(O)OH | 1 | CH₂ |
| 2.017 | H | H | H | H | —NHS(O)OH | 1 | CH₂ |
| 2.018 | H | H | H | H | —NMeS(O)OH | 1 | CH₂ |
| 2.019 | H | H | H | H | —NHS(O)₂CF₃ | 1 | CH₂ |
| 2.020 | H | H | H | H | —S(O)₂NHC(O)Me | 1 | CH₂ |
| 2.021 | H | H | H | H | —OS(O)₂NHC(O)Me | 1 | CH₂ |
| 2.022 | H | H | H | H | —NHS(O)₂NHC(O)Me | 1 | CH₂ |
| 2.023 | H | H | H | H | —NMeS(O)₂NHC(O)Me | 1 | CH₂ |
| 2.024 | H | H | H | H | —P(O)(OH)(OMe) | 1 | CH₂ |
| 2.025 | H | H | H | H | —P(O)(OH)(OH) | 1 | CH₂ |
| 2.026 | H | H | H | H | —OP(O)(OH)(OMe) | 1 | CH₂ |
| 2.027 | H | H | H | H | —OP(O)(OH)(OH) | 1 | CH₂ |
| 2.028 | H | H | H | H | —NHP(O)(OH)(OMe) | 1 | CH₂ |
| 2.029 | H | H | H | H | —NHP(O)(OH)(OH) | 1 | CH₂ |
| 2.030 | H | H | H | H | —NMeP(O)(OH)(OMe) | 1 | CH₂ |
| 2.031 | H | H | H | H | —NMeP(O)(OH)(OH) | 1 | CH₂ |
| 2.032 | H | H | H | H | —tetrazole | 1 | CH₂ |
| 2.033 | H | H | H | H | —S(O)₂OH | 2 | CH₂CH(NH₂) |
| 2.034 | H | H | H | H | —C(O)OH | 2 | CH₂CH(NH₂) |
| 2.035 | H | H | H | H | —C(O)NHCN | 1 | CH₂ |
| 2.036 | H | H | H | H | —OC(O)NHCN | 1 | CH₂ |
| 2.037 | H | H | H | H | —NHC(O)NHCN | 1 | CH₂ |
| 2.038 | H | H | H | H | —NMeC(O)NHCN | 1 | CH₂ |
| 2.039 | H | H | H | H | —S(O)₂NHCN | 1 | CH₂ |
| 2.040 | H | H | H | H | —OS(O)₂NHCN | 1 | CH₂ |
| 2.041 | H | H | H | H | —NHS(O)₂NHCN | 1 | CH₂ |
| 2.042 | H | H | H | H | —NMeS(O)₂NHCN | 1 | CH₂ |
| 2.043 | H | H | H | H | —S(O)₂NHS(O)₂Me | 1 | CH₂ |
| 2.044 | H | H | H | H | —OS(O)₂NHS(O)₂Me | 1 | CH₂ |
| 2.045 | H | H | H | H | —NHS(O)₂NHS(O)₂Me | 1 | CH₂ |
| 2.046 | H | H | H | H | —NMeS(O)₂NHS(O)₂Me | 1 | CH₂ |
| 2.047 | H | H | H | H | —P(O)H(OH) | 1 | CH₂ |
| 2.048 | H | H | H | H | —N(OH)C(O)Me | 1 | CH₂ |
| 2.049 | H | H | H | H | —ONHC(O)Me | 1 | CH₂ |

TABLE 3

| Compound | | | | | | | |
|---|---|---|---|---|---|---|---|
| number | R³ | R^{3a} | R⁴ | R⁵ | Z | m | Q |
| 3.001 | H | H | H | H | —C(O)OH | 2 | CH₂CH₂ |
| 3.002 | H | H | H | H | —C(O)OMe | 2 | CH₂CH₂ |
| 3.003 | H | H | H | H | —C(O)NHOMe | 2 | CH₂CH₂ |
| 3.004 | H | H | H | H | —OC(O)NHOMe | 2 | CH₂CH₂ |
| 3.005 | H | H | H | H | —NHC(O)NHOMe | 2 | CH₂CH₂ |
| 3.006 | H | H | H | H | —NMeC(O)NHOMe | 2 | CH₂CH₂ |
| 3.007 | H | H | H | H | —C(O)NHS(O)₂Me | 2 | CH₂CH₂ |
| 3.008 | H | H | H | H | —OC(O)NHS(O)₂Me | 2 | CH₂CH₂ |
| 3.009 | H | H | H | H | —NHC(O)NHS(O)₂Me | 2 | CH₂CH₂ |
| 3.010 | H | H | H | H | —NMeC(O)NHS(O)₂Me | 2 | CH₂CH₂ |
| 3.011 | H | H | H | H | —S(O)₂OH | 2 | CH₂CH₂ |
| 3.012 | H | H | H | H | —OS(O)₂OH | 2 | CH₂CH₂ |
| 3.013 | H | H | H | H | —NHS(O)₂OH | 2 | CH₂CH₂ |
| 3.014 | H | H | H | H | —NMeS(O)₂OH | 2 | CH₂CH₂ |
| 3.015 | H | H | H | H | —S(O)OH | 2 | CH₂CH₂ |
| 3.016 | H | H | H | H | —OS(O)OH | 2 | CH₂CH₂ |
| 3.017 | H | H | H | H | —NHS(O)OH | 2 | CH₂CH₂ |
| 3.018 | H | H | H | H | —NMeS(O)OH | 2 | CH₂CH₂ |
| 3.019 | H | H | H | H | —NHS(O)₂CF₃ | 2 | CH₂CH₂ |
| 3.020 | H | H | H | H | —S(O)₂NHC(O)Me | 2 | CH₂CH₂ |
| 3.021 | H | H | H | H | —OS(O)₂NHC(O)Me | 2 | CH₂CH₂ |
| 3.022 | H | H | H | H | —NHS(O)₂NHC(O)Me | 2 | CH₂CH₂ |
| 3.023 | H | H | H | H | —NMeS(O)₂NHC(O)Me | 2 | CH₂CH₂ |
| 3.024 | H | H | H | H | —P(O)(OH)(OMe) | 2 | CH₂CH₂ |
| 3.025 | H | H | H | H | —P(O)(OH)(OH) | 2 | CH₂CH₂ |
| 3.026 | H | H | H | H | —OP(O)(OH)(OMe) | 2 | CH₂CH₂ |
| 3.027 | H | H | H | H | —OP(O)(OH)(OH) | 2 | CH₂CH₂ |
| 3.028 | H | H | H | H | —NHP(O)(OH)(OMe) | 2 | CH₂CH₂ |
| 3.029 | H | H | H | H | —NHP(O)(OH)(OH) | 2 | CH₂CH₂ |
| 3.030 | H | H | H | H | —NMeP(O)(OH)(OMe) | 2 | CH₂CH₂ |
| 3.031 | H | H | H | H | —NMeP(O)(OH)(OH) | 2 | CH₂CH₂ |
| 3.032 | H | H | H | H | —tetrazole | 2 | CH₂CH₂ |
| 3.033 | H | H | H | H | —S(O)₂OH | 3 | CH₂CH₂CH(NH₂) |
| 3.034 | H | H | H | H | —C(O)OH | 3 | CH₂CH₂CH(NH₂) |
| 3.035 | H | H | H | H | —C(O)NHCN | 2 | CH₂CH₂ |
| 3.036 | H | H | H | H | —OC(O)NHCN | 2 | CH₂CH₂ |
| 3.037 | H | H | H | H | —NHC(O)NHCN | 2 | CH₂CH₂ |
| 3.038 | H | H | H | H | —NMeC(O)NHCN | 2 | CH₂CH₂ |
| 3.039 | H | H | H | H | —S(O)₂NHCN | 2 | CH₂CH₂ |
| 3.040 | H | H | H | H | —OS(O)₂NHCN | 2 | CH₂CH₂ |
| 3.041 | H | H | H | H | —NHS(O)₂NHCN | 2 | CH₂CH₂ |
| 3.042 | H | H | H | H | —NMeS(O)₂NHCN | 2 | CH₂CH₂ |
| 3.043 | H | H | H | H | —S(O)₂NHS(O)₂Me | 2 | CH₂CH₂ |
| 3.044 | H | H | H | H | —OS(O)₂NHS(O)₂Me | 2 | CH₂CH₂ |
| 3.045 | H | H | H | H | —NHS(O)₂NHS(O)₂Me | 2 | CH₂CH₂ |
| 3.046 | H | H | H | H | —NMeS(O)₂NHS(O)₂Me | 2 | CH₂CH₂ |
| 3.047 | H | H | H | H | —P(O)H(OH) | 2 | CH₂CH₂ |
| 3.048 | H | H | H | H | —N(OH)C(O)Me | 2 | CH₂CH₂ |
| 3.049 | H | H | H | H | —ONHC(O)Me | 2 | CH₂CH₂ |

This table discloses 49 specific compounds (3.001 to 3.049) of the formula (T-3):

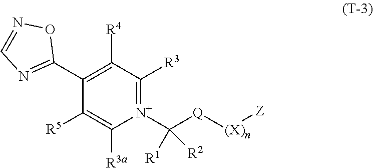

wherein m, Q, R³, R^{3a}, R⁴, R⁵ and Z are as defined in Table 3, R¹ and R² are hydrogen and n is 0.

### Table 4:

This table discloses 53 specific compounds (4.001 to 4.053) of the formula (T-4):

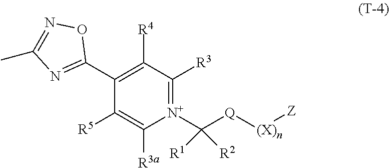

wherein m, Q, R³, R^{3a}, R⁴, R⁵ and Z are as defined above in Table 1, R¹ and R² are hydrogen and n is 0.

### Table 5:

This table discloses 49 specific compounds (5.001 to 5.049) of the formula (T-5):

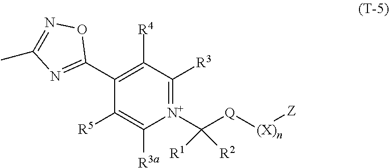

wherein m, Q, R³, R^{3a}, R⁴, R⁵ and Z are as defined above in Table 2, R¹ and R² are hydrogen and n is 0.

### Table 6:

This table discloses 49 specific compounds (6.001 to 6.049) of the formula (T-6):

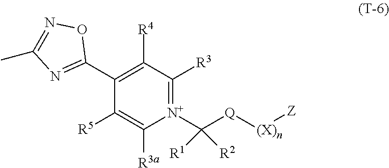

wherein m, Q, R³, R³¹, R⁴, R⁵ and Z are as defined above in Table 3, R¹ and R² are hydrogen and n is 0.

### Table 7:

This table discloses 53 specific compounds (7.001 to 7.053) of the formula (T-7):

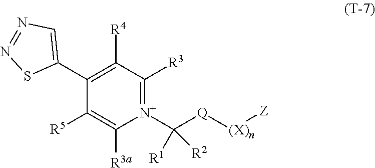

wherein m, Q, R³, R^{3a}, R⁴, R⁵ and Z are as defined above in Table 1, R¹ and R² are hydrogen and n is 0.

### Table 8:

This table discloses 49 specific compounds (8.001 to 8.049) of the formula (T-8):

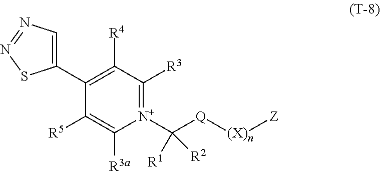

wherein m, Q, R³, R^{3a}, R⁴, R⁵ and Z are as defined above in Table 2, R¹ and R² are hydrogen and n is 0.

### Table 9:

This table discloses 49 specific compounds (9.001 to 9.049) of the formula (T-9):

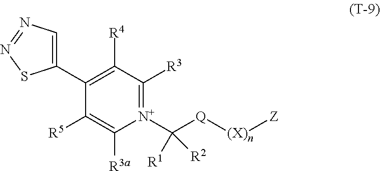

wherein m, Q, R³, R³¹, R⁴, R⁵ and Z are as defined above in Table 3, R¹ and R² are hydrogen and n is 0.

### Table 10:

This table discloses 53 specific compounds (10.001 to 10.053) of the formula (T-10):

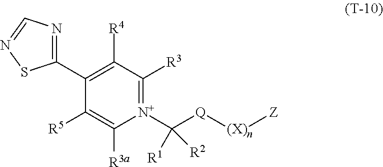

wherein m, Q, R³, R^{3a}, R⁴, R⁵ and Z are as defined above in Table 1, R¹ and R² are hydrogen and n is 0.

### Table 11:

This table discloses 49 specific compounds (11.001 to 11.049) of the formula (T-11):

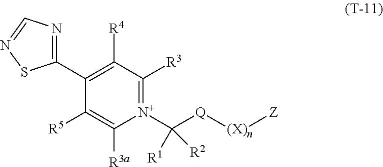

wherein m, Q, R³, R^{3a}, R⁴, R⁵ and Z are as defined above in Table 2, R¹ and R² are hydrogen and n is 0.

### Table 12:

This table discloses 49 specific compounds (12.001 to 12.049) of the formula (T-12):

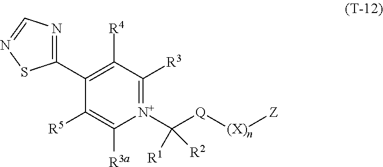

wherein m, Q, R³, R³¹, R⁴, R⁵ and Z are as defined above in Table 3, R¹ and R² are hydrogen and n is 0.

### Table 13:

This table discloses 53 specific compounds (13.001 to 13.053) of the formula (T-13):

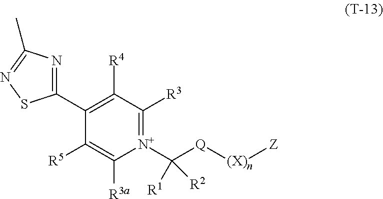

wherein m, Q, R³, R^{3a}, R⁴, R⁵ and Z are as defined above in Table 1, R¹ and R² are hydrogen and n is 0.

### Table 14:

This table discloses 49 specific compounds (14.001 to 14.049) of the formula (T-14):

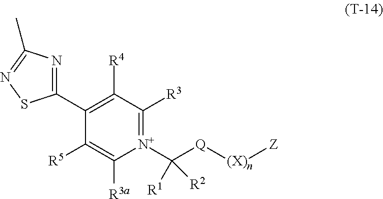

wherein m, Q, R³, R^{3a}, R⁴, R⁵ and Z are as defined above in Table 2, R¹ and R² are hydrogen and n is 0.

### Table 15:

This table discloses 49 specific compounds (15.001 to 15.049) of the formula (T-15):

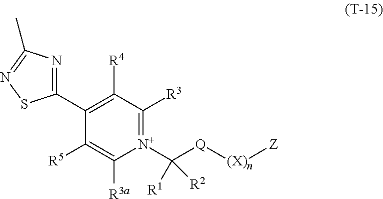

wherein m, Q, R³, R^{3a}, R⁴, R⁵ and Z are as defined above in Table 3, R¹ and R² are hydrogen and n is 0.

### Table 16:

This table discloses 53 specific compounds (16.001 to 16.053) of the formula (T-16):

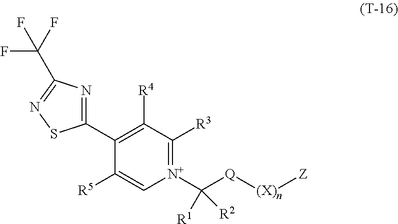

wherein m, Q, R³, R^{3a}, R⁴, R⁵ and Z are as defined above in Table 1, R¹ and R² are hydrogen and n is 0.

### Table 17:

This table discloses 49 specific compounds (17.001 to 17.049) of the formula (T-17):

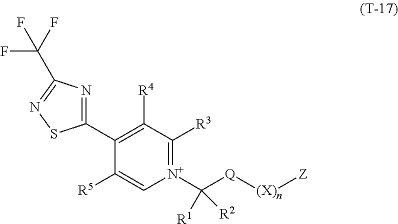

wherein m, Q, R³, R^{3a}, R⁴, R⁵ and Z are as defined above in Table 2, R¹ and R² are hydrogen and n is 0.

### Table 18:

This table discloses 49 specific compounds (18.001 to 18.049) of the formula (T-18):

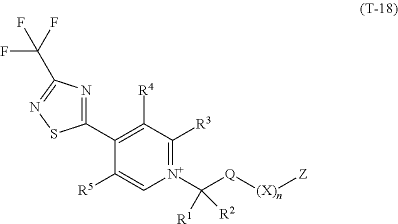

wherein m, Q, R³, R^{3a}, R⁴, R⁵ and Z are as defined above in Table 3, R¹ and R² are hydrogen and n is 0.

### Table 19:

This table discloses 53 specific compounds (19.001 to 19.053) of the formula (T-19):

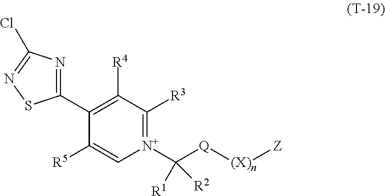

wherein m, Q, R³, R^{3a}, R⁴, R⁵ and Z are as defined above in Table 1, R¹ and R² are hydrogen and n is 0.

### Table 20:

This table discloses 49 specific compounds (20.001 to 20.049) of the formula (T-20):

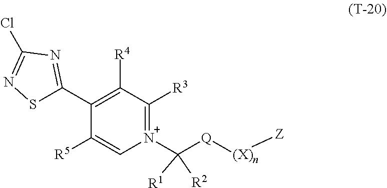

wherein m, Q, R³, R³¹, R⁴, R⁵ and Z are as defined above in Table 2, R¹ and R² are hydrogen and n is 0.

### Table 21:

This table discloses 49 specific compounds (21.001 to 21.049) of the formula (T-21):

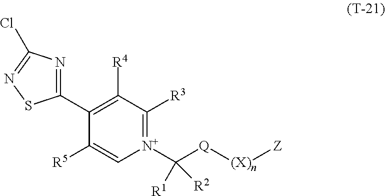

wherein m, Q, R³, R^{3a}, R⁴, R⁵ and Z are as defined above in Table 3, R¹ and R² are hydrogen and n is 0.

### Table 22:

This table discloses 53 specific compounds (22.001 to 22.053) of the formula (T-22):

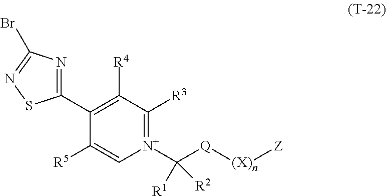

wherein m, Q, R³, R^{3a}, R⁴, R⁵ and Z are as defined above in Table 1, R¹ and R² are hydrogen and n is 0.

### Table 23:

This table discloses 49 specific compounds (23.001 to 23.049) of the formula (T-23):

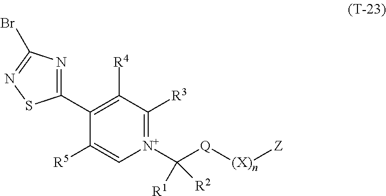

wherein m, Q, R³, R^{3a}, R⁴, R⁵ and Z are as defined above in Table 2, R¹ and R² are hydrogen and n is 0.

### Table 24:

This table discloses 49 specific compounds (24.001 to 24.049) of the formula (T-24):

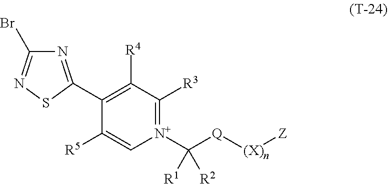

wherein m, Q, R³, R^{3a}, R⁴, R⁵ and Z are as defined above in Table 3, R¹ and R² are hydrogen and n is 0.

### Table 25:

This table discloses 53 specific compounds (25.001 to 25.053) of the formula (T-25):

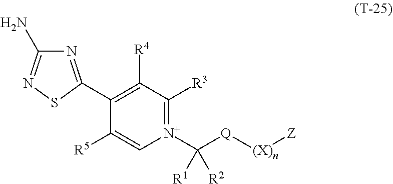

wherein m, Q, R³, R^{3a}, R⁴, R⁵ and Z are as defined above in Table 1, R¹ and R² are hydrogen and n is 0.

### Table 26:

This table discloses 49 specific compounds (26.001 to 26.049) of the formula (T-26):

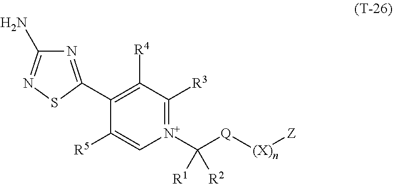

wherein m, Q, R³, R^{3a}, R⁴, R⁵ and Z are as defined above in Table 2, R¹ and R² are hydrogen and n is 0.

### Table 27:

This table discloses 49 specific compounds (27.001 to 27.049) of the formula (T-27):

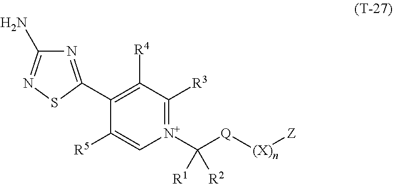

wherein m, Q, R³, R^{3a}, R⁴, R⁵ and Z are as defined above in Table 3, R¹ and R² are hydrogen and n is 0.

The compounds of the present invention may be prepared according to the following schemes in which the substituents n, m, r, A, Q, X, Z, R¹, R², R^{1a}, R^{2b}, R², R³, R^{3a}, R⁴, R⁵, R⁶, R⁷, R^{7a}, R^{7b}, R^{7c}, R⁸, R⁹, R¹⁰, R¹¹, R¹², R¹³, R¹⁴, R¹⁵, R^{15a}, R¹⁶, R¹⁷ and R¹⁸ are as defined hereinbefore unless explicitly stated otherwise. The compounds of the preceeding Tables 1 to 27 may thus be obtained in an analogous manner.

The compounds of formula (I) may be prepared by the alkylation of compounds of formula (X), wherein R³, R^{3a}, R⁴, R⁵ and A are as defined for compounds of formula (I), with a suitable alkylating agent of formula (W), wherein R¹, R², Q, X, n and Z are as defined for compounds of formula (I) and LG is a suitable leaving group, for example, halide or pseudohalide such as triflate, mesylate or tosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluroacetic acid at a temperature between −78° C. and 150° C. An alkylating agent of formula (W) may include, but is not limited to, bromoacetic acid, methyl bromoacetate, 3-bromopropionoic acid, methyl 3-bromopropionate, 2-bromo-N-methoxyacetamide, sodium 2-bromoethanesulphonate, 2,2-dimethylpropyl 2-(trifluoromethylsulfonyloxy)ethanesulfonate, 2-bromo-N-methanesulfonylacetamide, 3-bromo-N-methanesulfonylpropanamide, dimethoxyphosphorylmethyl trifluoromethanesulfonate, dimethyl 3-bromopropylphosphonate, 3-chloro-2,2-dimethyl-propanoic acid and diethyl 2-bromoethylphosphonate. Such alkylating agents and related compounds are either known in the literature or may be prepared by known literature methods. Compounds of formula (I) which may be described as esters of N-alkyl acids, which include, but are not limited to, esters of carboxylic acids, phosphonic acids, phosphinic acids, sulfonic acids and sulfinic acids, may be subsequently partially or fully hydrolysed by treament with a suitable reagent, for example, aqueous hydrochloric acid or trimethylsilyl bromide, in a suitable solvent at a suitable temperature between 0° C. and 100° C.

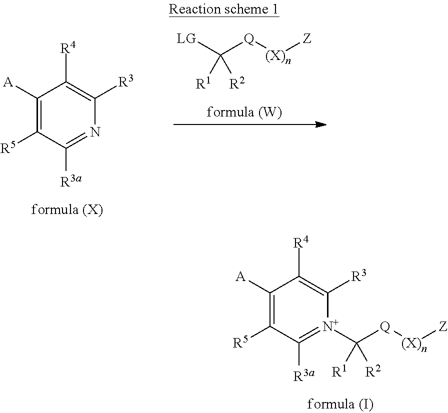

Additionally, compounds of formula (I) may be prepared by reacting compounds of formula (X), wherein R³, R^{3a}, R⁴, R⁵ and A are as defined for compounds of formula (I), with a suitably activated electrophilic alkene of formula (B), wherein Z is —S(O)₂OR¹⁰, —P(O)(R¹³)(OR¹⁰) or —C(O)OR¹⁰ and R¹, R², R^{1a}, R¹⁰ and R¹³ are as defined for compounds of formula (I), in a suitable solvent at a suitable temperature. Compounds of formula (B) are known in the literature, or may be prepared by known methods. Example reagents include, but are not limited to, acrylic acid, methacrylic acid, crotonic acid, 3,3-dimethylacrylic acid, methyl acrylate, ethene sulfonic acid, isopropyl ethylenesulfonate, 2,2-dimethylpropyl ethenesulfonate and dimethyl vinylphosphonate. The direct products of these reactions, which may be described as esters of N-alkyl acids, which include, but are not limited to, esters of carboxylic acids, phosphonic acids, phosphinic acids, sulfonic acids and sulfinic acids, may be subsequently partially or fully hydrolysed by treament with a suitable reagent in a suitable solvent at a suitable temperature, as described in reaction scheme 2.

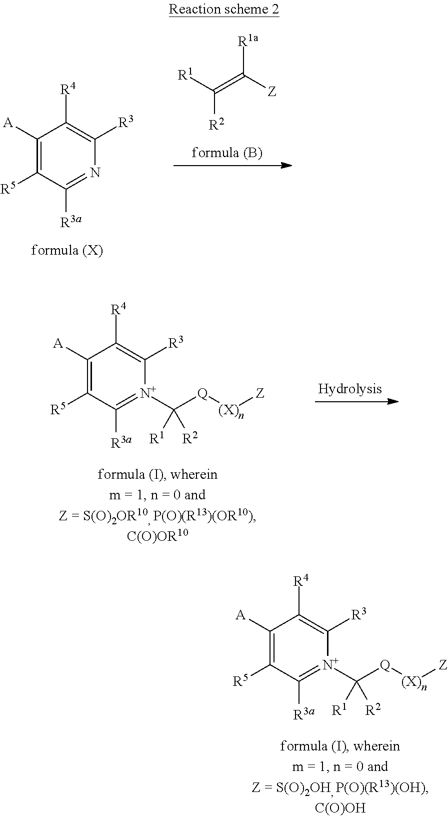

In a related reaction compounds of formula (I), wherein Q is C(R^{1a}R^{2b}), m is 1, 2 or 3, n=0 and Z is —S(O)₂OH, —OS(O)₂OH or —NR⁶S(O)₂OH, may be prepared by the reaction of compounds of formula (X), wherein R³, R^{3a}, R⁴, R⁵ and A are as defined for compounds of formula (I), with a cyclic alkylating agent of formula (E), (F) or (AF), wherein Y^{a} is C(R^{1a}R^{2b}), 0 or NR⁶ and R¹, R², Ria and R^{2b} are as defined for compounds of formula (I), in a suitable solvent at a suitable temperature, as described in reaction scheme 3. Suitable solvents and suitable temperatures are as previously described. An alkylating agent of formula (E) or (F) may include, but is not limited to, 1,3-propanesultone, 1,4-butanesultone, ethylenesulfate, 1,3-propylene sulfate and 1,2,3-oxathiazolidine 2,2-dioxide. Such alkylating agents and related compounds are either known in the literature or may be prepared by known literature methods.

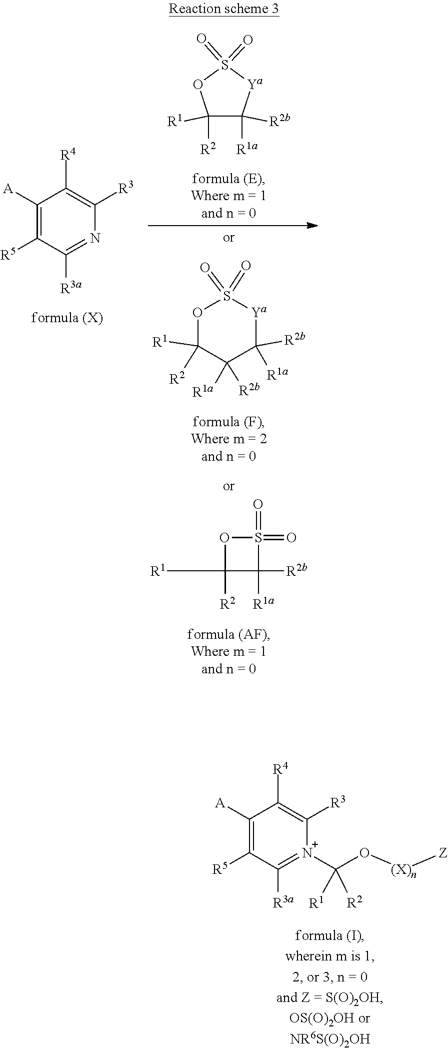

A compound of formula (I), wherein m is 0, n is 0 and Z is —S(O)₂OH, may be prepared from a compound of formula (I), wherein m is 0, n is 0 and Z is C(O)OR¹⁰, by treatment with trimethylsilylchlorosulfonate in a suitable solvent at a suitable temperature, as described in reaction scheme 4. Preferred conditions include heating the carboxylate precursor in neat trimethylsilylchlorosulfonate at a temperature between 25° C. and 150° C.

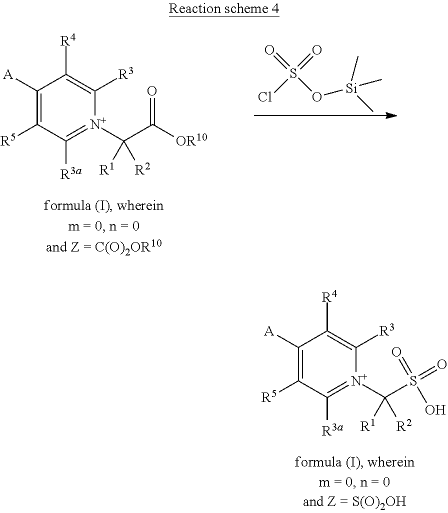

Furthermore, compounds of formula (I) may be prepared by reacting compounds of formula (X), wherein R³, R^{3a}, R⁴, R⁵ and A are as defined for compounds of formula (I), with a suitable alcohol of formula (WV), wherein R¹, R², Q, X, n and Z are as defined for compounds of formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 5. Such alcohols are either known in the literature or may be prepared by known literature methods.

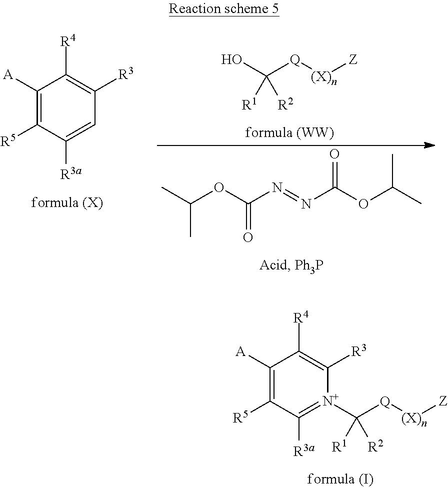

In another approach a compound of formula (I), wherein n, Q, Z, X, R¹, R², R³, R^{3a}, R⁴, R⁵ and A are as defined for compounds of formula (I), may be prepared from a compound of formula (R) and an oxidant, in a suitable solvent at a suitable temperature, as outlined in reaction scheme 6. Example oxidants include, but are not limited to, 2,3-dichloro-5,6-dicyano-1,4-benzoquinone, tetrachloro-p-benzoquinone, potassium permanganate, manganese dioxide, 2,2,6,6-tetramethyl-1-piperidinyloxy and bromine. Related reactions are known in the literature.

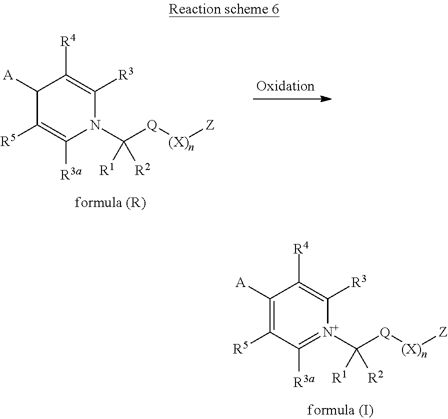

A compound of formula (R), wherein n, Q, Z, X, R¹, R², R³, R^{3a}, R⁴, R⁵ and A are as defined for compounds of formula (I), may be prepared from a compound of formula (S) and an organometallic of formula (T), wherein M′ includes, but is not limited to, organomagnesium, organolithium, organocopper and organozinc reagents, in a suitable solvent at a suitable temperature, optionally in the presence of an additional transition metal additive, as outlined in reaction scheme 7. Example conditions include treating a compound of formula (S) with a Grignard of formula (T), in the presence of 0.05-100 mol % copper iodide, in a solvent such as tetrahydrofuran at a temperature between −78° C. and 100° C. Organometallics of formula (T) are known in the literature, or may be prepared by known literature methods. Compounds of formula (S) may be prepared by analogous reactions to those for the preparation of compounds of formula (I) from a compound of formula (XX).

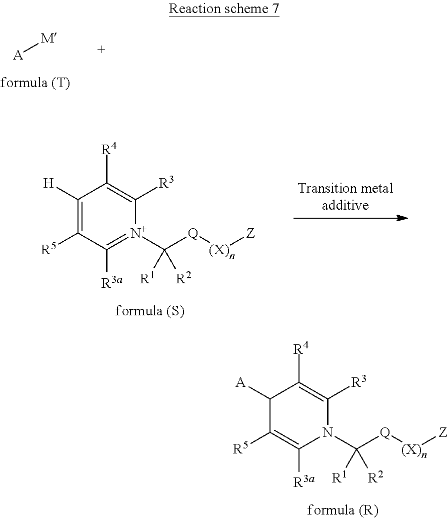

Biaryl pyridines of formula (X) are known in the literature or may be prepared using literature methods. Example methods include, but are not limited to, the transition metal cross-coupling of compounds of formula (H) and formula (J), or alternatively compounds of formula (K) and formula (L), in which compounds of formula (J) and formula (L), wherein M′ is either an organostannane, organoboronic acid or ester, organotrifluoroborate, organomagnesium, organocopper or organozinc, as outlined in reaction scheme 8. Hal is defined as a halogen or pseudo halogen, for example triflate, mesylate and tosylate. Such cross-couplings include Stille (for example Sauer, J.; Heldmann, D. K. Tetrahedron, 1998, 4297), Suzuki-Miyaura (for example Luebbers, T.; Flohr, A.; Jolidon, S.; David-Pierson, P.; Jacobsen, H.; Ozmen, L.; Baumann, K. Bioorg. Med. Chem. Lett., 2011, 6554), Negishi (for example Imahori, T.; Suzawa, K.; Kondo, Y. Heterocycles, 2008, 1057), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Compounds of formula (H), formula (K) and formula (L) are known in the literature, or may be prepared by known literature methods.

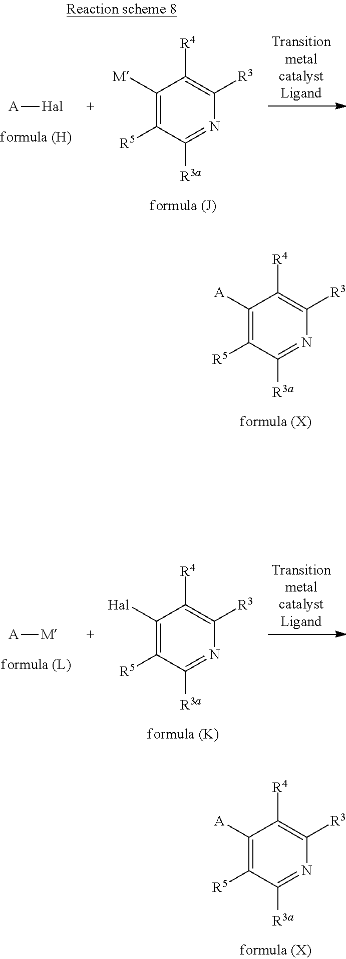

A compound of formula (J), wherein M′ is either an organostannane, organoboronic acid or ester, organotrifluoroborate, organomagnesium, organocopper or organozinc, may be prepared from a compound of formula (XX), wherein R³, R^{3a}, R⁴ and R⁵ are as defined for compounds of formula (I), by metalation, as outlined in reaction scheme 9. Similar reactions are known in the literature (for example Ramphal et al, WO2015/153683, Unsinn et al., Organic Letters, 15(5), 1128-1131; 2013, Sadler et al., Organic & Biomolecular Chemistry, 12(37), 7318-7327; 2014. Alternatively, an organometallic of formula (J) may be prepared from compounds of formula (K), wherein R³, R^{3a}, R⁴ and R⁵ are as defined for compounds of formula (I), and Hal is defined as a halogen or pseudo halogen, for example triflate, mesylate and tosylate, as described in scheme 9. Example conditions to prepare an compound of formula (J) wherein M′ is an organostannane, include treatment of a compound of formula (K) with lithium tributyl tin in an appropriate solvent at an appropriate temperature (for example see WO 2010/038465). Example conditions to prepare compound of formula (J) wherein M′ is an organoboronic acid or ester, include treatment of a compound of formula (K) with bis(pinacolato)diboron, in the presence of an appropriate transition metal catalyst, appropriate ligand, appropriate base, in an appropriate solvent at an appropriate temperature (for example KR 2015135626). Compounds of formula (K) and formula (XX) are either known in the literature or can be prepared by known methods.

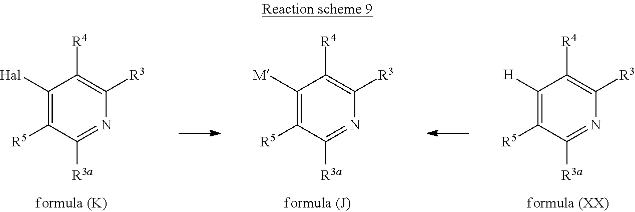

In an additional approach, outlined in scheme 10, biaryl pyridines of formula (X) may be prepared by classical ring synthesis approaches starting from a compound of formula (ZZ), wherein R³, R^{3a}, R⁴ and R⁵ are as defined for compounds of formula (I) and T is a functional group which can be converted through one or more chemical steps into a 5-membered heteroaryl A, wherein A is as defined for compounds of formula (I). Such functional groups include, but are not limited to, acid, ester, nitrile, amide, thioamide and ketone. Related transformations are known in the literature. Substituted pyridines may be prepared using methodology outlined in the literature.

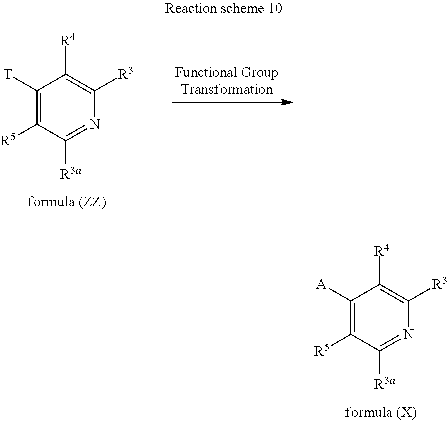

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations. The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

Emulsifiable concentrates:
active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%
Suspension concentrates:
active ingredient: 5 to 75%, preferably 10 to 50%
water: 94 to 24%, preferably 88 to 30%
surface-active agent: 1 to 40%, preferably 2 to 30%
Wettable powders:
active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)):—I+acetochlor, I+acifluorfen (including acifluorfen-sodium), I+aclonifen, I+ametryn, I+amicarbazone, I+aminopyralid, I+aminotriazole, I+atrazine, I+beflubutamid-M, I+bensulfuron (including bensulfuron-methyl), I+bentazone, I+bicyclopyrone, I+bilanafos, I+bispyribac-sodium, I+bixlozone, I+bromacil, I+bromoxynil, I+butachlor, I+butafenacil, I+carfentrazone (including carfentrazone-ethyl), I+cloransulam (including cloransulam-methyl), 1+chlorimuron (including chlorimuron-ethyl), I+chlorotoluron, I+chlorsulfuron, I+cinmethylin, I+clacyfos, I+clethodim, I+clodinafop (including clodinafop-propargyl), I+clomazone, I+clopyralid, I+cyclopyranil, I+cyclopyrimorate, I+cyclosulfamuron, I+cyhalofop (including cyhalofop-butyl), I+2,4-D (including the choline salt and 2-ethylhexyl ester thereof), I+2,4-DB, I+desmedipham, I+dicamba (including the aluminium, aminopropyl, bis-aminopropylmethyl, choline, dichloroprop, diglycolamine, dimethylamine, dimethylammonium, potassium and sodium salts thereof) I+diclosulam, I+diflufenican, I+diflufenzopyr, I+dimethachlor, I+dimethenamid-P, I+diquat dibromide, diuron, I+ethalfluralin, I+ethofumesate, I+fenoxaprop (including fenoxaprop-P-ethyl), I+fenoxasulfone, I+fenquinotrione, I+fentrazamide, I+flazasulfuron, I+florasulam, I+florpyrauxifen (including florpyrauxifen-benzyl), I+fluazifop (including fluazifop-P-butyl), I+flucarbazone (including flucarbazone-sodium), I+flufenacet, I+flumetsulam, I+flumioxazin, I+fluometuron, I+flupyrsulfuron (including flupyrsulfuron-methyl-sodium), I+fluroxypyr (including fluroxypyr-meptyl), I+fomesafen, I+foramsulfuron, I+glufosinate (including the ammonium salt thereof), I+glyphosate (including the diammonium, isopropylammonium and potassium salts thereof), I+halauxifen (including halauxifen-methyl), I+haloxyfop (including haloxyfop-methyl), I+hexazinone, I+hydantocidin, I+imazamox, I+imazapic, I+imazapyr, I+imazethapyr, I+indaziflam, I+iodosulfuron (including iodosulfuron-methyl-sodium), I+iofensulfuron (including iofensulfuron-sodium), I+ioxynil, I+isoproturon, I+isoxaflutole, I+lancotrione, I+MCPA, I+MCPB, I+mecoprop-P, I+mesosulfuron (including mesosulfuron-methyl), I+mesotrione, I+metamitron, I+metazachlor, I+methiozolin, I+metolachlor, I+metosulam, I+metribuzin, I+metsulfuron, I+napropamide, I+nicosulfuron, I+norflurazon, I+oxadiazon, I+oxasulfuron, I+oxyfluorfen, I+paraquat dichloride, I+pendimethalin, I+penoxsulam, I+phenmedipham, I+picloram, I+pinoxaden, I+pretilachlor, I+primisulfuron-methyl, I+prometryne, I+propanil, I+propaquizafop, I+propyrisulfuron, I+propyzamide, I+prosulfocarb, I+prosulfuron, I+pyraclonil, I+pyraflufen (including pyraflufen-ethyl), I+pyrasulfotole, I+pyridate, I+pyriftalid, I+pyrimisulfan, I+pyroxasulfone, I+pyroxsulam, I+quinclorac, I+quinmerac, I+quizalofop (including quizalofop-P-ethyl and quizalofop-P-tefuryl), I+rimsulfuron, I+saflufenacil, I+sethoxydim, I+simazine, I+S-metalochlor, I+sulfentrazone, I+sulfosulfuron, I+tebuthiuron, I+tefuryltrione, I+tembotrione, I+terbuthylazine, I+terbutryn, I+tetflupyrolimet, I+thiencarbazone, I+thifensulfuron, I+tiafenacil, I+tolpyralate, I+topramezone, I+tralkoxydim, I+triafamone, I+triallate, I+triasulfuron, I+tribenuron (including tribenuron-methyl), I+triclopyr, I+trifloxysulfuron (including trifloxysulfuron-sodium), I+trifludimoxazin, I+trifluralin, I+triflusulfuron, I+ethyl 2-[[3-[2-chloro-4-fluoro-5-[3-methyl-2,6-dioxo-4-(trifluoromethyl)pyrimidin-1-yl]phenoxy]-2-pyridyl]oxy]acetate, I+3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, I+4-hydroxy-1-methoxy-5-methyl-3-[4-(trifluoromethyl)-2-pyridyl]imidazolidin-2-one, I+4-hydroxy-1,5-dimethyl-3-[4-(trifluoromethyl)-2-pyridyl]imidazolidin-2-one, I+5-ethoxy-4-hydroxy-1-methyl-3-[4-(trifluoromethyl)-2-pyridyl]imidazolidin-2-one, I+4-hydroxy-1-methyl-3-[4-(trifluoromethyl)-2-pyridyl]imidazolidin-2-one, I+4-hydroxy-1,5-dimethyl-3-[1-methyl-5-(trifluoromethyl)pyrazol-3-yl]imidazolidin-2-one, I+(4R)₁-(5-tert-butylisoxazol-3-yl)-4-ethoxy-5-hydroxy-3-methyl-imidazolidin-2-one, I+3-[2-(3,4-dimethoxyphenyl)-6-methyl-3-oxo-pyridazine-4-carbonyl]bicyclo[3.2.1]octane-2,4-dione, I+2-[2-(3,4-dimethoxyphenyl)-6-methyl-3-oxo-pyridazine-4-carbonyl]-5-methyl-cyclohexane-1,3-dione, I+2-[2-(3,4-dimethoxyphenyl)-6-methyl-3-oxo-pyridazine-4-carbonyl]cyclohexane-1,3-dione, I+2-[2-(3,4-dimethoxyphenyl)-6-methyl-3-oxo-pyridazine-4-carbonyl]-5,5-dimethyl-cyclohexane-1,3-dione, I+6-[2-(3,4-dimethoxyphenyl)-6-methyl-3-oxo-pyridazine-4-carbonyl]-2,2,4,4-tetramethyl-cyclohexane-1,3,5-trione, I+2-[2-(3,4-dimethoxyphenyl)-6-methyl-3-oxo-pyridazine-4-carbonyl]-5-ethyl-cyclohexane-1,3-dione, I+2-[2-(3,4-dimethoxyphenyl)-6-methyl-3-oxo-pyridazine-4-carbonyl]-4,4,6,6-tetramethyl-cyclohexane-1,3-dione, I+2-[6-cyclopropyl-2-(3,4-dimethoxyphenyl)-3-oxo-pyridazine-4-carbonyl]-5-methyl-cyclohexane-1,3-dione, I+3-[6-cyclopropyl-2-(3,4-dimethoxyphenyl)-3-oxo-pyridazine-4-carbonyl]bicyclo[3.2.1]octane-2,4-dione, I+2-[6-cyclopropyl-2-(3,4-dimethoxyphenyl)-3-oxo-pyridazine-4-carbonyl]-5,5-dimethyl-cyclohexane-1,3-dione, I+6-[6-cyclopropyl-2-(3,4-dimethoxyphenyl)-3-oxo-pyridazine-4-carbonyl]-2,2,4,4-tetramethyl-cyclohexane-1,3,5-trione, I+2-[6-cyclopropyl-2-(3,4-dimethoxyphenyl)-3-oxo-pyridazine-4-carbonyl]cyclohexane-1,3-dione, I+4-[2-(3,4-dimethoxyphenyl)-6-methyl-3-oxo-pyridazine-4-carbonyl]-2,2,6,6-tetramethyl-tetrahydropyran-3,5-dione, I+4-[6-cyclopropyl-2-(3,4-dimethoxyphenyl)-3-oxo-pyridazine-4-carbonyl]-2,2,6,6-tetramethyl-tetrahydropyran-3,5-dione, I+4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate).

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include:—I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; 1+furilazole I+isoxadifen (including isoxadifen-ethyl); I+mefenpyr (including mefenpyr-diethyl); I+metcamifen and I+oxabetrinil.

Particularly preferred are mixtures of a compound of formula (I) with cyprosulfamide, isoxadifen (including isoxadifen-ethyl), cloquintocet (including cloquintocet-mexyl) and/or metcamifen.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Formulation Examples

Wettable powders

| | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

### Emulsifiable Concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of | 4% |
| ethylene oxide) | |
| Cyclohexanone | 30% |
| xylene mixture | 50% |

Emulsions of any required dilution, which can be used in plant protection, can be obtained from this concentrate by dilution with water.

Dusts

| | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruder Granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

### Coated Granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### List of Abbreviations

Boc=tert-butyloxycarbonyl
br=broad
CDCl₃=chloroform-d
CD₃OD=methanol-d
° C.=degrees Celsius
D₂O=water-d
DCM=dichloromethane
d=doublet
dd=double doublet
dt=double triplet
DMSO=dimethylsulfoxide
EtOAc=ethyl acetate
h=hour(s)
HCl=hydrochloric acid
HPLC=high-performance liquid chromatography (description of the apparatus and the methods used for HPLC are given below)
m=multiplet
M=molar
min=minutes
MHz=mega hertz
mL=millilitre
mp=melting point
ppm=parts per million
q=quartet
quin=quintet
rt=room temperature
s=singlet
t=triplet
THE=tetrahydrofuran

### LC/MS=Liquid Chromatography Mass Spectrometry

### Preparative Reverse Phase HPLC Method:

Compounds purified by mass directed preparative HPLC using ES+/ES− on a Waters FractionLynx Autopurification system comprising a 2767 injector/collector with a 2545 gradient pump, two 515 isocratic pumps, SFO, 2998 photodiode array (Wavelength range (nm): 210 to 400), 2424 ELSD and QDa mass spectrometer. A Waters Atlantis T3 5 micron 19×10 mm guard column was used with a Waters Atlantis T3 OBD, 5 micron 30×100 mm prep column.

Ionisation method: Electrospray positive and negative: Cone (V) 20.00, Source Temperature (° C.) 120, Cone Gas Flow (L/Hr.) 50

Mass range (Da): positive 100 to 800, negative 115 to 800.

The preparative HPLC was conducted using an 11.4 minute run time (not using at column dilution, bypassed with the column selector), according to the following gradient table:

| Time (mins) | Solvent A (%) | Solvent B (%) | Flow (ml/min) |
|---|---|---|---|
| 0.00 | 100 | 0 | 35 |
| 2.00 | 100 | 0 | 35 |
| 2.01 | 100 | 0 | 35 |
| 7.0 | 90 | 10 | 35 |
| 7.3 | 0 | 100 | 35 |
| 9.2 | 0 | 100 | 35 |
| 9.8 | 99 | 1 | 35 |
| 11.35 | 99 | 1 | 35 |
| 11.40 | 99 | 1 | 35 |

515 pump 0 ml/min Acetonitrile (ACD)
515 pump 1 ml/min 90% Methanol/10% Water (make up pump)
Solvent A: Water with 0.05% Trifluoroacetic Acid
Solvent B: Acetonitrile with 0.05% Trifluoroacetic Acid

### Preparation Examples

### Example 1: Preparation of 2-[4-[3-(trifluoromethyl)-1,2,4-thiadiazol-5-yl]pyridin-1-ium-1-yl]acetic acid 2,2,2-trifluoroacetate A1

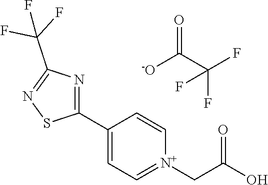

### Step 1: Preparation of 4-(4,4,5,5-tetramethyl-1,3,2-dioxaborolan-2-yl)-1,2,3,6-tetrahydropyridin-1-ium chloride

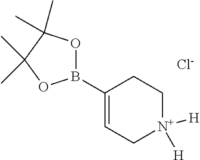

A mixture of tert-butyl 4-(4,4,5,5-tetramethyl-1,3,2-dioxaborolan-2-yl)-3,6-dihydro-2H-pyridine-1-carboxylate (2 g) and aqueous hydrochloric acid (4M in 1,4-dioxane, 19 mL) was stirred at room temperature overnight. The reaction mixture was concentrated and the residue was triturated with ether to give 4-(4,4,5,5-tetramethyl-1,3,2-dioxaborolan-2-yl)-1,2,3,6-tetrahydropyridin-1-ium chloride as a white solid.

1H NMR (400 MHz, D₂O) 6.28-6.21 (m, 1H), 3.69-3.62 (m, 2H), 3.21 (t, 2H), 2.41-2.24 (m, 2H), 1.12 (s, 12H) (NH proton missing)

### Step 2: Preparation of tert-butyl 2-[4-(4,4,5,5-tetramethyl-1,3,2-dioxaborolan-2-yl)-3,6-dihydro-2H-pyridin-1-yl]acetate

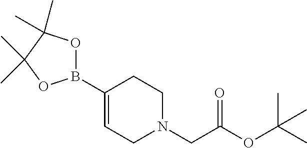 [SMILES: CC(C)(C)OC(=O)CN1CC=C(B2OC(C)(C)C(C)(C)O2)CC1]

A mixture of 4-(4,4,5,5-tetramethyl-1,3,2-dioxaborolan-2-yl)-1,2,3,6-tetrahydropyridin-1-ium chloride (1.89 g), acetonitrile (31.6 mL), potassium carbonate (2.62 g) and tert-butyl 2-chloroacetate (1.35 mL) was heated at 90° C. for 20 hours. The reaction mixture was cooled and partitioned between water and dichloromethane, then further extracted with dichloromethane (×2). The combined organic phase was dried over magnesium sulfate and concentrated to give tert-butyl 2-[4-(4,4,5,5-tetramethyl-1,3,2-dioxaborolan-2-yl)-3,6-dihydro-2H-pyridin-1-yl]acetate as a yellow gum.

1H NMR (400 MHz, CDCl₃) 6.52-6.43 (m, 1H), 3.24-3.15 (m, 4H), 2.68 (t, 2H), 2.34-2.26 (m, 2H), 1.46 (s, 9H), 1.25 (s, 12H)

### Step 3: Preparation of tert-butyl 2-[4-[3-(trifluoromethyl)-1,2,4-thiadiazol-5-yl]-3,6-dihydro-2H-pyridin-1-yl]acetate

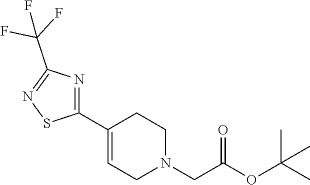 [SMILES: CC(C)(C)OC(=O)CN1CC=C(c2nc(C(F)(F)F)ns2)CC1]

A mixture of tert-butyl 2-[4-(4,4,5,5-tetramethyl-1,3,2-dioxaborolan-2-yl)-3,6-dihydro-2H-pyridin-1-yl]acetate (0.838 g), [1,1′-bis(diphenylphosphino)ferrocene]dichloropalladium(II) (0.155 g), 5-chloro-3-(trifluoromethyl)-1,2,4-thiadiazole (0.4 g), sodium carbonate (0.899 g), 1,4-dioxane (7.43 mL) and water (7.43 mL) was degassed with nitrogen then heated at 120° C. under microwave irradiation for 1 hour. The reaction mixture was cooled to room temperature then filtered through diatomaceous earth and partitioned between water and ethyl acetate. The organic phase was concentrated then purified by silica gel chromatography eluting with 0 to 50% ethyl acetate in cyclohexane to give tert-butyl 2-[4-[3-(trifluoromethyl)-1,2,4-thiadiazol-5-yl]-3,6-dihydro-2H-pyridin-1-yl]acetate.

1H NMR (400 MHz, CDCl₃) 6.83-6.99 (m, 1H), 3.44-3.50 (m, 2H), 3.30-3.33 (m, 2H), 2.90-2.96 (m, 2H), 2.69-2.75 (m, 2H), 1.48-1.51 (m, 9H)

### Step 4: Preparation of 2-[4-[3-(trifluoromethyl)-1,2,4-thiadiazol-5-yl]pyridin-1-ium-1-yl]acetic acid 2,2,2-trifluoroacetate A1

To a solution of tert-butyl 2-[4-[3-(trifluoromethyl)-1,2,4-thiadiazol-5-yl]-3,6-dihydro-2H-pyridin-1-yl]acetate (0.43 g) in 1,4-dioxane (6.6 mL) was added N-bromosuccinimide (0.294 g) in one portion followed by stirring at room temperature for 2 hours. To the reaction mixture was then added aqueous hydrochloric acid (2.4 mL, 4M in 1,4-dioxane) with additional stirring for 5 hours at room temperature. The reaction mixture was diluted with tert-butyl methyl ether (20 mL) and the resulting solid was filtered, washed with additional tert-butyl methyl ether then purified by preparative reverse phase HPLC (trifluoroacetic acid was present in the eluent) to afford 2-[4-[3-(trifluoromethyl)-1,2,4-thiadiazol-5-yl]pyridin-1-ium-1-yl]acetic acid 2,2,2-trifluoroacetate.

1H NMR (400 MHz, D₂O) 9.06 (d, 2H), 8.69 (d, 2H), 5.41 (s, 2H) (CO₂H proton missing)

### Example 2: Preparation of [4-(3-methyl-1,2,4-thiadiazol-5-yl)pyridin-1-ium-1-yl]methanesulfonate A2

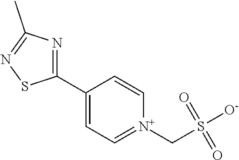 [SMILES: Cc1nsc(-c2cc[n+](CS(=O)(=O)[O-])cc2)n1]

### Step 1: Preparation of (NE)-N-[1-(dimethylamino)ethylidene]pyridine-4-carbothioamide

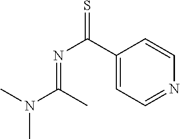

A mixture of pyridine-4-carbothioamide (1 g) and 1,1-dimethoxy-N,N-dimethyl-ethanamine (1.05 mL) were stirred together at room temperature for one hour. The reaction was concentrated to give (NE)-N-[1-(dimethylamino)ethylidene]pyridine-4-carbothioamide as a red gum which crystallised on standing.

1H NMR (400 MHz, CD₃OD) 8.55-8.51 (m, 2H), 8.11-8.08 (m, 2H), 3.37 (s, 3H), 3.26-3.21 (m, 3H), 2.52 (s, 3H)

### Step 2: Preparation of 3-methyl-5-(4-pyridyl)-1,2,4-thiadiazole

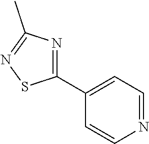

To a solution of (NE)-N-[1-(dimethylamino)ethylidene]pyridine-4-carbothioamide (1.5 g) and pyridine (1.23 mL) in ethanol (36 mL) at room temperature was added a second solution of hydroxylamine-O-sulfonic acid (0.9 g) in methanol (14 mL). The reaction mixture was stirred at room temperature for one hour then quenched with saturated aqueous sodium bicarbonate and extracted with dichloromethane. The organic phase was concentrated, triturated with hexane then dried to give 3-methyl-5-(4-pyridyl)-1,2,4-thiadiazole as a brown solid.

1H NMR (400 MHz, CD₃OD) 8.78-8.71 (m, 2H), 8.00-7.96 (m, 2H), 2.72 (s, 3H)

### Step 3: Preparation of methyl 2-[4-(3-methyl-1,2,4-thiadiazol-5-yl)pyridin-1-ium-1-yl]acetate bromide

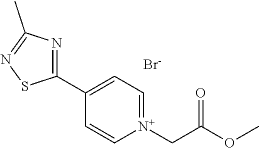 [SMILES: COC(=O)C[n+]1ccc(-c2nc(C)ns2)cc1 ; [Br-]]

A mixture of 3-methyl-5-(4-pyridyl)-1,2,4-thiadiazole (1.165 g), acetonitrile (20 mL) and methyl 2-bromoacetate (0.93 mL) was heated at 80° C. for 25 hours. The reaction mixture was partitioned between water and dichloromethane and the aqueous phase was concentrated to give methyl 2-[4-(3-methyl-1,2,4-thiadiazol-5-yl)pyridin-1-ium-1-yl]acetate bromide which was used without further purification.

### Step 4: Preparation of 2-[4-(3-methyl-1,2,4-thiadiazol-5-yl)pyridin-1-ium-1-yl]acetic acid chloride A59

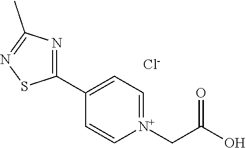 [SMILES: Cc1nsc(-c2cc[n+](CC(=O)O)cc2)n1 ; [Cl-]]

A solution of crude methyl 2-[4-(3-methyl-1,2,4-thiadiazol-5-yl)pyridin-1-ium-1-yl]acetate bromide (1.7 g) and 2M aqueous hydrochloric acid (45 mL) was heated at 50° C. for 4 hours. The reaction mixture was then concentrated to give 2-[4-(3-methyl-1,2,4-thiadiazol-5-yl)pyridin-1-ium-1-yl]acetic acid chloride.

1H NMR (400 MHz, D₂O) 8.93-8.89 (m, 2H), 8.51-8.46 (m, 2H), 5.37 (s, 2H), 2.67 (s, 3H) (CO₂H proton missing)

### Step 5: Preparation of [4-(3-methyl-1,2,4-thiadiazol-5-yl)pyridin-1-ium-1-yl]methanesulfonate A2

A mixture of 2-[4-(3-methyl-1,2,4-thiadiazol-5-yl)pyridin-1-ium-1-yl]acetic acid chloride (0.83 g) and trimethylsilyl chlorosulfonate (11 mL) was heated at 120° C. overnight. The reaction mixture was cooled to room temperature and partitioned between water and dichloromethane. The aqueous phase was then concentrated and purified by preparative reverse phase HPLC to afford [4-(3-methyl-1,2,4-thiadiazol-5-yl)pyridin-1-ium-1-yl]methanesulfonate as a white solid.

1H NMR (400 MHz, D₂O) 9.02-9.20 (m, 2H) 8.55-8.68 (m, 2H) 5.58-5.81 (m, 2H) 2.65-2.82 (m, 3H)

### Example 3: Preparation of [4-(thiadiazol-5-yl)pyridin-1-ium-1-yl]methanesulfonate A3

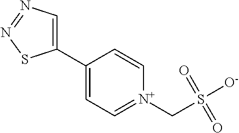 [SMILES: O=S(=O)([O-])C[n+]1ccc(-c2cnns2)cc1]

### Step 1: Preparation of methyl 2-diazo-3-oxo-3-(4-pyridyl)propanoate

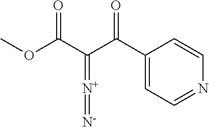

To a mixture of methyl 3-oxo-3-(4-pyridyl)propanoate (4 g) and 4-acetamidobenzenesulfonyl azide (6.082 g) in dichloromethane (130 mL) was added triethylamine (9.43 mL) drop wise at 0° C. The reaction mixture was slowly warmed to room temperature then stirred overnight. After filtration through silica, followed by and washing with dichloromethane, the filtrate was then concentrated to afford methyl 2-diazo-3-oxo-3-(4-pyridyl)propanoate as a yellow gum.

1H NMR (400 MHz, CDCl₃) 8.78-8.71 (m, 2H), 7.45-7.40 (m, 2H) 3.79 (s, 3H)

### Step 2: Preparation of methyl 5-(4-pyridyl)thiadiazole-4-carboxylate

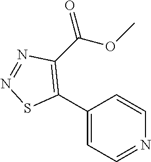

A mixture of methyl 2-diazo-3-oxo-3-(4-pyridyl)propanoate (4.58 g) and Lawesson's reagent (11.2 g) in toluene (112 mL) was heated at 120° C. overnight. The reaction mixture was concentrated then purified by silica gel chromatography eluting with 0 to 50% methanol in dichloromethane to give methyl 5-(4-pyridyl)thiadiazole-4-carboxylate which was used in the next step without further purification.

### Step 3: Preparation of 5-(4-pyridyl)thiadiazole-4-carboxylic acid

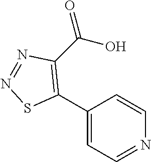 [SMILES: O=C(O)c1nnsc1-c1ccncc1]

A mixture of crude methyl 5-(4-pyridyl)thiadiazole-4-carboxylate (4 g) and 2M aqueous hydrochloric acid (150 mL) was heated at reflux for 3 hours. The reaction mixture was concentrated and partitioned between water and ethyl acetate. The aqueous layer was concentrated to give 5-(4-pyridyl)thiadiazole-4-carboxylic acid which was used in the next step without further purification.

### Step 4: Preparation of 5-pyridin-1-ium-4-ylthiadiazole 2,2,2-trifluoroacetate

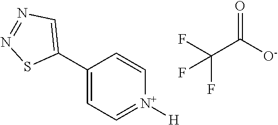 [SMILES: c1cc(-c2cnns2)cc[nH+]1 ; O=C([O-])C(F)(F)F]

A mixture of 5-(4-pyridyl)thiadiazole-4-carboxylic acid (3.35 g), water (5 mL) and 1,4-dioxane (80 mL) was heated at 100° C. overnight. The reaction mixture was concentrated and purified by preparative reverse phase HPLC (trifluoroacetic acid was present in the eluent) to afford 5-pyridin-1-ium-4-ylthiadiazole 2,2,2-trifluoroacetate as a white solid.

1H NMR (400 MHz, CD₃OD) 9.45 (s, 1H), 8.90-8.83 (m, 2H), 8.21-8.16 (m, 2H)

### Step 5: Preparation of methyl 2-[4-(thiadiazol-5-yl)pyridin-1-ium-1-yl]acetate bromide A8

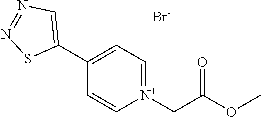 [SMILES: COC(=O)C[n+]1ccc(-c2cnns2)cc1 ; [Br-]]

A mixture of 5-pyridin-1-ium-4-ylthiadiazole 2,2,2-trifluoroacetate (0.5 g), acetonitrile (10 mL) and methyl 2-bromoacetate (0.44 mL) was heated at 80° C. for 25 hours. The reaction mixture was concentrated and partitioned between water and dichloromethane. The aqueous layer was concentrated to give methyl 2-[4-(thiadiazol-5-yl)pyridin-1-ium-1-yl]acetate bromide as a brown gum.

1H NMR (400 MHz, D₂O) 9.39-9.45 (m, 1H) 8.85-8.93 (m, 2H) 8.35-8.44 (m, 2H) 5.55 (s, 2H) 3.73-3.84 (m, 3H)

### Step 6: Preparation of 2-[4-(thiadiazol-5-yl)pyridin-1-ium-1-yl]acetic acid chloride A9

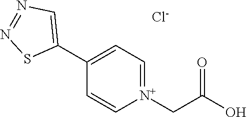 [SMILES: O=C(O)C[n+]1ccc(-c2cnns2)cc1 ; [Cl-]]

A mixture of methyl 2-[4-(thiadiazol-5-yl)pyridin-1-ium-1-yl]acetate bromide (0.46 g) and 2M aqueous hydrochloric acid (20 mL) was heated at 50° C. for 5 hours. The reaction mixture was concentrated to give 2-[4-(thiadiazol-5-yl)pyridin-1-ium-1-yl]acetic acid chloride.

1H NMR (400 MHz, D₂O) 9.38-9.43 (m, 1H) 8.83-8.90 (m, 2H) 8.33-8.40 (m, 2H) 5.40 (s, 2H) (CO₂H proton missing)

### Step 7: Preparation of [4-(thiadiazol-5-yl)pyridin-1-ium-1-yl]methanesulfonate A3

A mixture of 2-[4-(thiadiazol-5-yl)pyridin-1-ium-1-yl]acetic acid chloride (0.46 g) and trimethylsilyl chlorosulfonate (5.4 mL) was heated at 120° C. overnight. The reaction mixture was cooled to room temperature and partitioned between water and dichloromethane. The aqueous phase was concentrated then purified by preparative reverse phase HPLC to afford [4-(thiadiazol-5-yl)pyridin-1-ium-1-yl]methanesulfonate as a white solid.

1H NMR (400 MHz, D₂O) 9.41-9.49 (m, 1H) 8.95-9.04 (m, 2H) 8.37-8.49 (m, 2H) 5.68 (br s, 2H)

### Example 4: Preparation of 2-[4-(1,2,4-thiadiazol-5-yl)pyridin-1-ium-1-yl]ethanesulfonate A5

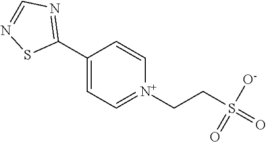 [SMILES: O=S(=O)([O-])CC[n+]1ccc(-c2ncns2)cc1]

A mixture of 5-(4-pyridyl)-1,2,4-thiadiazole (300 mg), sodium 2-bromoethanesulfonic acid (465 mg) and water (6 mL) was heated at 100° C. overnight. The reaction mixture was concentrated and purified by preparative reverse phase HPLC to afford 2-[4-(1,2,4-thiadiazol-5-yl)pyridin-1-ium-1-yl]ethanesulfonate

1H NMR (400 MHz, D₂O) 9.02-9.11 (m, 2H) 8.87-8.99 (m, 1H) 8.47-8.60 (m, 2H) 4.94-5.05 (m, 2H) 3.48-3.61 (m, 2H)

### Example 5: Preparation of 3-[4-(1,2,4-thiadiazol-5-yl)pyridin-1-ium-1-yl]propanoic acid chloride A7

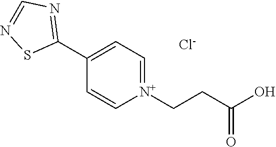 [SMILES: O=C(O)CC[n+]1ccc(-c2ncns2)cc1 ; [Cl-]]

### Step 1: Preparation of methyl 3-[4-(1,2,4-thiadiazol-5-yl)pyridin-1-ium-1-yl]propanoate 2,2,2-trifluoroacetate

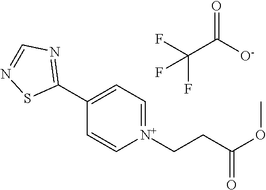 [SMILES: COC(=O)CC[n+]1ccc(-c2ncns2)cc1 ; O=C([O-])C(F)(F)F]

A mixture of 5-(4-pyridyl)-1,2,4-thiadiazole (0.3 g), acetonitrile (6 mL) and methyl 3-bromopropionate (0.31 mL) was heated at 80° C. for 25 hours. The reaction mixture was cooled and partitioned between water and dichloromethane. The aqueous layer was concentrated then purified by preparative reverse phase HPLC to afford methyl 3-[4-(1,2,4-thiadiazol-5-yl)pyridin-1-ium-1-yl]propanoate 2,2,2-trifluoroacetate as a white solid

1H NMR (400 MHz, D₂O) 9.07 (d, 2H), 8.94 (s, 1H), 8.54 (d, 2H), 4.90 (t, 2H), 3.60 (s, 3H), 3.18 (t, 2H)

### Step 2: Preparation of 3-[4-(1,2,4-thiadiazol-5-yl)pyridin-1-ium-1-yl]propanoic acid chloride A7

A mixture of methyl 3-[4-(1,2,4-thiadiazol-5-yl)pyridin-1-ium-1-yl]propanoate (50 mg) and 2M aqueous hydrochloric acid (1 mL) was heated at 50° C. for 5 hours. The reaction mixture was concentrated to give 3-[4-(1,2,4-thiadiazol-5-yl)pyridin-1-ium-1-yl]propanoic acid chloride as a white solid.

1H NMR (400 MHz, D₂O) 9.05 (d, 2H), 8.92 (s, 1H), 8.51 (d, 2H), 4.87 (t, 2H), 3.15 (t, 2H) (CO₂H proton missing)

### Example 6: Preparation of [4-(1,2,4-thiadiazol-5-yl)pyridin-1-ium-1-yl]methanesulfonate A4

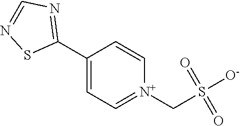 [SMILES: O=S(=O)([O-])C[n+]1ccc(-c2ncns2)cc1]

### Step 1: Preparation of tert-butyl 4-(1,2,4-thiadiazol-5-yl)-3,6-dihydro-2H-pyridine-1-carboxylate

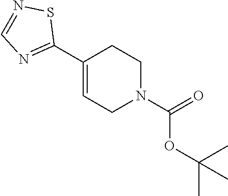 [SMILES: CC(C)(C)OC(=O)N1CC=C(c2ncns2)CC1]

To a mixture of 5-bromo-1,2,4-thiadiazole (5 g), 1,4-dioxane (50 mL) and water (17 mL) was added tert-butyl 4-(4,4,5,5-tetramethyl-1,3,2-dioxaborolan-2-yl)-3,6-dihydro-2H-pyridine-1-carboxylate (10 g), cesium carbonate (15 g) and 1,1′-bis(diphenylphosphino)ferrocene-palladium(II)dichloride dichloromethane complex (2.5 g). The mixture was purged with nitrogen then heated at 95° C. for 19 hours. The mixture was filtered through celite and the filtrate was concentrated and purified by silica gel chromatography eluting with 0 to 30% ethyl acetate in cyclohexane to give tert-butyl 4-(1,2,4-thiadiazol-5-yl)-3,6-dihydro-2H-pyridine-1-carboxylate as a yellow liquid.

¹H NMR (400 MHz, CDCl₃) 8.61 (s, 1H), 6.81 (br s, 1H), 4.18 (br d, 2H), 3.68 (br t, 2H), 2.68 (br dd, 2H), 1.49 (s, 9H)

### Step 2: Preparation of 5-(1,2,3,6-tetrahydropyridin-4-yl)-1,2,4-thiadiazole hydrochloride

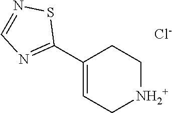 [SMILES: C1=C(c2ncns2)CC[NH2+]C1 ; [Cl-]]

A mixture of tert-butyl 4-(1,2,4-thiadiazol-5-yl)-3,6-dihydro-2H-pyridine-1-carboxylate (5 g) and hydrochloric acid (4M in dioxane, 26 mL) was stirred at room temperature for 1.5 hours. The mixture was concentrated, azeotroped with toluene and the resulting residue was washed with ethyl acetate (2×40 mL) and dried to give 5-(1,2,3,6-tetrahydropyridin-4-yl)-1,2,4-thiadiazole hydrochloride as a pale pink solid.

¹H NMR (400 MHz, D₂O) 8.63 (s, 1H), 6.80 (tt, 1H), 3.90 (q, 2H), 3.46 (t, 2H), 2.85 (qt, 2H)

### Step 3: Preparation of sodium [4-(1,2,4-thiadiazol-5-yl)-3,6-dihydro-2H-pyridin-1-yl]methanesulfonate

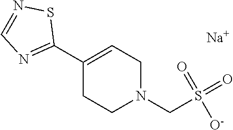 [SMILES: O=S(=O)([O-])CN1CC=C(c2ncns2)CC1 ; [Na+]]

To a solution of 5-(1,2,3,6-tetrahydropyridin-4-yl)-1,2,4-thiadiazole hydrochloride (2.5 g) in water (12 mL) was added sodium hydroxide (0.54 g). Sodium hydroxymethanesulfonate (formaldehyde sodium bisulfite adduct, 1.6 g) was added and the mixture was stirred at room temperature for 1 hour, periodically checking the pH of the solution was maintained between pH 10 and 11 by the addition of further aqueous 2M sodium hydroxide. The mixture was freeze dried to give sodium [4-(1,2,4-thiadiazol-5-yl)-3,6-dihydro-2H-pyridin-1-yl]methanesulfonate as an off-white solid, which was used without further purification.

¹H NMR (400 MHz, D₂O) 8.61 (s, 1H), 6.86 (td, 1H), 3.90 (s, 2H), 3.66 (q, 2H), 3.15 (t, 2H), 2.67-2.62 (m, 2H)

### Step 4: Preparation of [4-(1,2,4-thiadiazol-5-yl)pyridin-1-ium-1-yl]methanesulfonate A4

To a mixture of sodium [4-(1,2,4-thiadiazol-5-yl)-3,6-dihydro-2H-pyridin-1-yl]methanesulfonate (2.452 g) in dry acetonitrile (15.8 mL), under nitrogen atmosphere, was added 2,3-dichloro-5,6-dicyano-1,4-benzoquinone (3.67 g) portion wise over 3 minutes. The mixture was stirred at 25° C. for 18 hours. To the mixture was added trimethylsilyl bromide (0.718 mL). After 15 minutes stirring tetrahydrofuran (47.4 mL) was added and the mixture stirred for a further 20 minutes. The solid was filtered off, washed with tetrahydrofuran and dried. The solid was purified by preparative reverse phase HPLC to give [4-(1,2,4-thiadiazol-5-yl)pyridin-1-ium-1-yl]methanesulfonate as an off-white solid.

¹H NMR (400 MHz, D₂O) 9.25-9.02 (m, 3H), 8.81-8.66 (m, 2H), 5.85-5.74 (m, 2H)

### Example 7: Preparation of 2-[4-(thiadiazol-4-yl)pyridin-1-ium-1-yl]ethyl sulfate A65

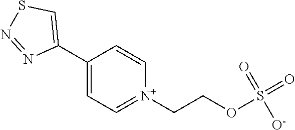 [SMILES: O=S(=O)([O-])OCC[n+]1ccc(-c2csnn2)cc1]

A mixture of 4-(4-pyridyl)thiadiazole (0.1 g), 1,3,2-dioxathiolane 2,2-dioxide (0.087 g) and 1,2-dichloroethane (6 mL) was heated at 85° C. for 18 hours. The resulting precipitate was filtered off and air dried to give 2-[4-(thiadiazol-4-yl)pyridin-1-ium-1-yl]ethyl sulfate as a white solid.

¹H NMR (400 MHz, DMSO-d₆) 10.23-10.36 (m, 1H), 9.09-9.24 (m, 2H), 8.73-8.96 (m, 2H), 4.73-4.94 (m, 2H), 4.18-4.34 (m, 2H)

### Example 8: Preparation of N-methyl-5-(4-pyridyl)isoxazole-3-carboxamide

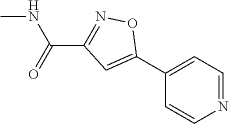

To a solution of 5-(4-pyridyl)isoxazole-3-carboxylic acid (0.5 g) in dichloromethane (14 mL) was added 1-chloro-N,N,2-trimethyl-prop-1-en-1-amine (0.35 mL) drop wise. After stirring for 30 minutes a solution of methanamine (2M in tetrahydrofuran, 2.63 mL) and N,N′-diisopropylethylamine (0.504 mL) in dichloromethane was added drop wise. The resulting mixture was stirred overnight at room temperature. The mixture was partitioned between dichloromethane and water. The organic layer was concentrated to give N-methyl-5-(4-pyridyl)isoxazole-3-carboxamide as a yellow solid.

¹H NMR (400 MHz, DMSO-d₆) 8.90-8.81 (m, 1H), 8.81-8.75 (m, 2H), 7.95-7.87 (m, 2H), 7.67 (s, 1H), 2.81 (d, 3H)

### Example 9: Preparation of 5-(4-pyridyl)-1,2,4-oxadiazole

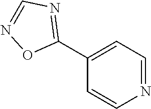 [SMILES: c1cc(-c2ncno2)ccn1]

### Step 1: Preparation of N-(dimethylaminomethylene)pyridine-4-carboxamide

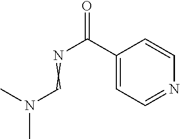

A mixture of pyridine-4-carboxamide (5 g) and 1,1-dimethoxy-N,N-dimethyl-methanamine (5.46 mL) were stirred together at room temperature for 4 hours. The mixture was concentrated and purified by silica gel chromatography eluting with 0 to 50% methanol in acetonitrile to give N-(dimethylaminomethylene)pyridine-4-carboxamide as a white solid.

¹H NMR (400 MHz, CDCl₃) 8.77-8.70 (m, 2H), 8.69-8.65 (m, 1H), 8.08-8.02 (m, 2H), 3.24 (d, 6H)

### Step 2: Preparation of 5-(4-pyridyl)-1,2,4-oxadiazole

To N-(dimethylaminomethylene)pyridine-4-carboxamide (3.99 g) was added a solution of dioxane (27 mL), hydroxylamine (50% aqueous solution, 2.07 mL) and acetic acid (32 mL). The mixture was heated at 90° C. for 1 hour. The reaction mixture was concentrated and partitioned between saturated aqueous sodium bicarbonate solution and dichloromethane. The organic layer was concentrated and purified by preparative reverse phase HPLC to give 5-(4-pyridyl)-1,2,4-oxadiazole as a white solid.

¹H NMR (400 MHz, CD₃OD) 8.95-8.88 (m, 3H), 8.30-8.25 (m, 2H)

### Example 10: Preparation of 4-(2-methyltetrazol-5-yl)pyridine

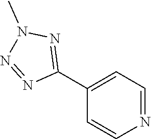

To a mixture of 4-(1H-tetrazol-5-yl)pyridine (0.4 g) and N,N-dimethylformamide (3.5 mL) was added dimethyl carbonate (2.2 mL) and 1,4-diazabicyclo[2.2.2]octane (0.032 g) and the mixture was heated at 130° C. overnight. The reaction mixture was cooled to room temperature and 0.25M aqueous sodium hydroxide (11 mL) was added and the mixture extracted with ethyl acetate (×3). The combined organic layers were washed with water and concentrated to give 4-(2-methyltetrazol-5-yl)pyridine.

¹H NMR (400 MHz, CDCl₃) 8.80-8.75 (m, 2H), 8.04-7.99 (m, 2H), 4.45 (s, 3H)

The material is isolated as a mixture with 4-(I-methyltetrazol-5-yl)pyridine.

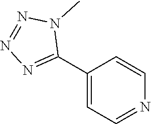

¹H NMR (400 MHz, CDCl₃) 8.91-8.85 (m, 2H), 7.72-7.68 (m, 2H), 4.26 (s, 3H) Separation of the isomers occurred after alkylation of the pyridine.

### Example 11: Preparation of methoxy-[[4-(1,2,4-thiadiazol-5-yl)pyridin-1-ium-1-yl]methyl]phosphinate A30

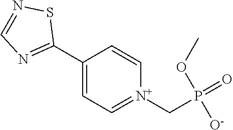 [SMILES: COP(=O)([O-])C[n+]1ccc(-c2ncns2)cc1]

### Step 1: Preparation of dimethoxyphosphorylmethanol

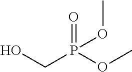

To a solution of methoxyphosphonoyloxymethane (8 g) in methanol (40 mL) was added paraformaldehyde (2.18 g) and potassium carbonate (0.502 g) at room temperature. The reaction mixture was stirred at room temperature for 1 hour. The reaction mixture was filtered through celite and washed with dichloromethane (50 mL). The filtrate was concentrated then purified by silica gel chromatography eluting with a mixture of ethyl acetate in n-hexanes to give dimethoxyphosphorylmethanol as colourless liquid.

¹H NMR (400 MHz, CDCl₃) 3.96-3.94 (d, 2H), 3.83-3.80 (d, 6H) (OH proton missing)

### Step 2: Preparation of dimethoxyphosphorylmethyl trifluoromethanesulfonate

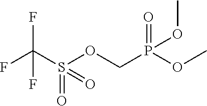

To a solution of dimethoxyphosphorylmethanol (5 g) in dichloromethane (50 mL) at −78° C., under nitrogen atmosphere, was added 2,6-lutidine (6.94 mL) and triflic anhydride (6.0 mL). The resulting reaction mixture was allowed to warm to room temperature and stirred at room temperature for 1 hour.

The reaction mixture was poured into water and extracted with dichloromethane (2×50 mL). The combined organic layers were washed with 1M aqueous hydrochloric acid (50 mL), dried over sodium sulfate and concentrated to afford dimethoxyphosphorylmethyl trifluoromethanesulfonate as pale yellow liquid.

¹H NMR (400 MHz, CDCl₃) 4.67-4.64 (d, 2H), 3.90-3.87 (d, 6H)

### Step 3: Preparation of 5-[1-(dimethoxyphosphorylmethyl)pyridin-1-ium-4-yl]-1,2,4-thiadiazole trifluoromethanesulfonate A77

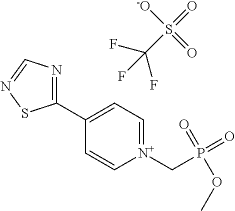

To a solution of 5-(4-pyridyl)-1,2,4-thiadiazole (1.5 g) in tetrahydrofuran (20 mL) was added dimethoxyphosphorylmethyl trifluoromethanesulfonate (3.56 g) at room temperature. The resulting mixture was heated at 60° C. for 16 hours. The reaction mixture was concentrated and the residue was dissolved in water (25 mL) and washed with dichloromethane (2×25 mL). The water layer was concentrated and purified by reverse phase HPLC (100% water) to afford 5-[1-(dimethoxyphosphorylmethyl)pyridin-1-ium-4-yl]-1,2,4-thiadiazole trifluoromethanesulfonate as a light brown solid.

¹H NMR (400 MHz, DMSO-d₆) 9.33 (s, 1H), 9.22-9.17 (m, 2H), 8.88-8.83 (m, 2H), 5.54 (d, 2H), 3.83-3.76 (m, 6H)

### Step 4: Preparation of methoxy-[[4-(1,2,4-thiadiazol-5-yl)pyridin-1-ium-1-yl]methyl]phosphinate A30

To a mixture of 5-[1-(dimethoxyphosphorylmethyl)pyridin-1-ium-4-yl]-1,2,4-thiadiazole trifluoromethanesulfonate (0.3 g) in dichloromethane (10 mL) was added bromotrimethylsilane (0.1 mL) at room temperature. The reaction mixture was stirred at room temperature for 2 hours. The reaction mixture was concentrated and the residue was dissolved in water (25 mL) and washed with dichloromethane (2×25 mL). The water layer was concentrated and purified by reverse phase HPLC (100% water) to give methoxy-[[4-(1,2,4-thiadiazol-5-yl)pyridin-1-ium-1-yl]methyl]phosphinate as an off-white solid.

¹H NMR (400 MHz, D₂O) 8.96-8.88 (m, 3H), 8.54 (d, 2H), 4.90-4.83 (m, 2H), 3.54 (d, 3H)

### Example 12: Preparation of hydroxy-[[4-(1,2,4-thiadiazol-5-yl)pyridin-1-ium-1-yl]methyl]phosphinate A32

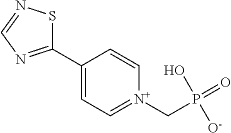 [SMILES: O=P([O-])(O)C[n+]1ccc(-c2ncns2)cc1]

To a solution of 5-[1-(dimethoxyphosphorylmethyl)pyridin-1-ium-4-yl]-1,2,4-thiadiazole trifluoromethanesulfonate (0.3 g) in dichloromethane (10 ml) was added bromotrimethylsilane (0.5 mlx) at room temperature. The reaction mixture was stirred at room temperature for 4 hours. The reaction mixture was concentrated and the residue was dissolved in water (25 ml) and washed with dichloromethane (2×25 mL). The water layer was concentrated and purified by reverse phase HPLC (100% water) to give hydroxy-[[4-(1,2,4-thiadiazol-5-yl)pyridin-1-ium-1-yl]methyl]phosphinate as a colourless gum.

¹H NMR (400 MHz, D₂O) 9.01-8.86 (m, 3H), 8.54 (d, 2H), 4.84-4.78 (m, 2H) (POH proton missing)

### Example 13: Preparation of methyl-[[4-(1,2,4-thiadiazol-5-yl)pyridin-1-ium-1-yl]methyl]phosphinate A10

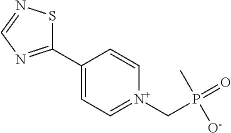 [SMILES: CP(=O)([O-])C[n+]1ccc(-c2ncns2)cc1]

### Step 1: Preparation of [ethoxy(methyl)phosphoryl]methanol

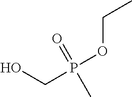

To a solution of 1-[ethoxy(methyl)phosphoryl]oxyethane (3 g) in acetonitrile (30 mL) was added triethylamine (0.668 g), paraformaldehyde (0.727 g) and water (0.436 mL) at room temperature. The reaction mixture was stirred at room temperature for 16 hours. The reaction mixture was concentrated and purified by silica gel chromatography eluting with a mixture of methanol in dichloromethane to give [ethoxy(methyl)phosphoryl]methanol as a colourless liquid.

¹H NMR (400 MHz, CDCl₃) 5.31-5.17 (m, 1H), 4.12-4.08 (m, 2H), 3.87-3.82 (m, 2H), 1.55-1.51 (d, 3H), 1.35-1.31 (t, 3H)

### Step 2: Preparation of [ethoxy(methyl)phosphoryl]methyl trifluoromethanesulfonate

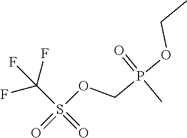

To a solution of [ethoxy(methyl)phosphoryl]methanol (2 g) in dichloromethane (30 mL) at −78° C., under nitrogen atmosphere, was added 2,6-lutidine (2.68 g) and triflic anhydride (2.8 mL). The resulting reaction mixture was allowed to warm to room temperature and stirred at room temperature for 3 hours. The reaction mixture was poured into water (50 mL) and extracted with dichloromethane (3×50 mL). The combined organic layers were washed with 1M aqueous hydrochloric acid (50 mL), dried over sodium sulfate and concentrated to afford [ethoxy(methyl)phosphoryl]methyl trifluoromethanesulfonate as light orange oil.

¹H NMR (400 MHz, CDCl₃) 4.71-4.55 (m, 2H), 4.24-4.10 (m, 2H), 1.68-1.64 (d, 3H), 1.40-1.37 (t, 3H)

### Step 3: Preparation of 5-[1-[[ethoxy(methyl)phosphoryl]methyl]pyridin-1-ium-4-yl]-1,2,4-thiadiazole trifluoromethanesulfonate A47

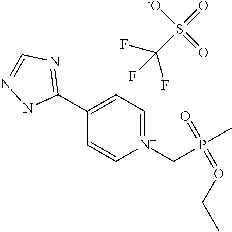

To a solution of 5-(4-pyridyl)-1,2,4-thiadiazole (1 g) in tetrahydrofuran (20 mL) was added [ethoxy(methyl)phosphoryl]methyl trifluoromethanesulfonate (1.67 g) at room temperature. The resulting mixture was heated at 65° C. for 16 hours. The reaction mixture was concentrated and the residue was dissolved in water (25 mL) and washed with dichloromethane (2×50 mL). The water layer was concentrated and purified by reverse phase HPLC (100% water) to afford 5-[1-[[ethoxy(methyl)phosphoryl]methyl]pyridin-1-ium-4-yl]-1,2,4-thiadiazole trifluoromethanesulfonate as a brown solid.

¹H NMR (400 MHz, DMSO-d₆) 9.33 (s, 1H), 9.16 (d, 2H), 8.85 (d, 2H), 5.37 (d, 2H), 4.14-4.03 (m, 2H), 1.69 (d, 3H), 1.27-1.19 (m, 3H)

### Step 4: Preparation of methyl-[[4-(1,2,4-thiadiazol-5-yl)pyridin-1-ium-1-yl]methyl]phosphinate A10

To a stirred solution of 5-[1-[[ethoxy(methyl)phosphoryl]methyl]pyridin-1-ium-4-yl]-1,2,4-thiadiazole trifluoromethanesulfonate (0.3 g) in dichloromethane (15 mL) was added bromotrimethylsilane (0.361 g) at room temperature. The reaction mixture was stirred at room temperature for 16 hours. The reaction mixture was concentrated and purified by reverse phase HPLC (100% water) to give methyl-[[4-(1,2,4-thiadiazol-5-yl)pyridin-1-ium-1-yl]methyl]phosphinate as a light green gum.

¹H NMR (400 MHz, CD₃OD) 9.08 (s, 3H), 8.77 (br d, 2H), 5.12 (br s, 2H), 1.71-1.46 (m, 3H)

### Example 14: Preparation of 3-[4-[5-(trifluoromethyl)-1,2,4-oxadiazol-3-yl]pyridin-1-ium-1-yl]propane-1-sulfonate A15

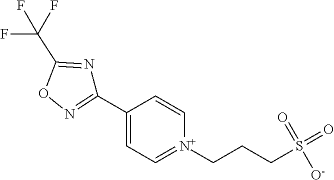 [SMILES: O=S(=O)([O-])CCC[n+]1ccc(-c2noc(C(F)(F)F)n2)cc1]

A mixture of 1,3-propanesultone (0.082 g) and 3-(4-pyridyl)-5-(trifluoromethyl)-1,2,4-oxadiazole (0.095 g) in 1,4-dioxane (2.2 mL) was heated at 100° C. for 20 hours. The resulting precipitate was filtered off and washed with acetone to give 3-[4-[5-(trifluoromethyl)-1,2,4-oxadiazol-3-yl]pyridin-1-ium-1-yl]propane-1-sulfonate as a colourless solid.

¹H NMR (400 MHz, D₂O) 9.09 (d, 2H), 8.65 (br d, 2H), 4.78-4.84 (m, 2H), 2.95 (t, 2H), 2.44 (br t, 2H)

### Example 15: Preparation of 3-[4-[3-(trifluoromethyl)-1,2,4-thiadiazol-5-yl]pyridin-1-ium-1-yl]propanoic acid acetate A48

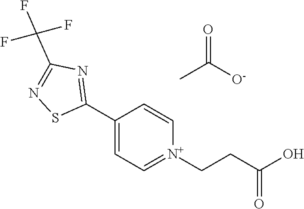

Step 1: Preparation of ethyl 3-[4-(4,4,5,5-tetramethyl-1,3,2-dioxaborolan-2-yl)-3,6-dihydro-2H-pyridin-1-yl]propanoate

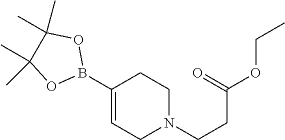 [SMILES: CCOC(=O)CCN1CC=C(B2OC(C)(C)C(C)(C)O2)CC1]

To a solution of 4-(4,4,5,5-tetramethyl-1,3,2-dioxaborolan-2-yl)-1,2,3,6-tetrahydropyridine hydrochloride (3 g) in acetonitrile (60 mL) was added potassium carbonate (5.065 g) followed by ethyl 3-bromopropanoate (1.644 mL). After heating at reflux for 16 hours the mixture was concentrated. The resulting residue was stirred in tert-butyl methyl ether, then filtered. The filtrate was concentrated to give ethyl 3-[4-(4,4,5,5-tetramethyl-1,3,2-dioxaborolan-2-yl)-3,6-dihydro-2H-pyridin-1-yl]propanoate, which was used without further purification.

### Step 2: Preparation of ethyl 3-[4-[3-(trifluoromethyl)-1,2,4-thiadiazol-5-yl]-3,6-dihydro-2H-pyridin-1-yl]propanoate

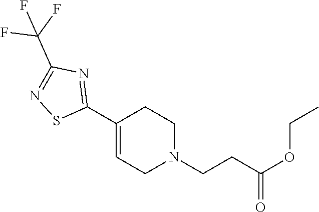 [SMILES: CCOC(=O)CCN1CC=C(c2nc(C(F)(F)F)ns2)CC1]

A mixture of ethyl 3-[4-(4,4,5,5-tetramethyl-1,3,2-dioxaborolan-2-yl)-3,6-dihydro-2H-pyridin-1-yl]propanoate (0.81 g), [1,1′-bis(diphenylphosphino)ferrocene]dichloropalladium(II) (0.175 g), 5-chloro-3-(trifluoromethyl)-1,2,4-thiadiazole (0.45 g), sodium carbonate (1.012 g), 1,4-dioxane (8.35 mL) and water (8.35 mL) was degassed with nitrogen then heated at 120° C. under microwave irradiation for 1 hour. The reaction mixture was cooled to room temperature then filtered through diatomaceous earth. The filtrate was concentrated then purified by silica gel chromatography eluting with a mixture of ethyl acetate in cyclohexane to give ethyl 3-[4-[3-(trifluoromethyl)-1,2,4-thiadiazol-5-yl]-3,6-dihydro-2H-pyridin-1-yl]propanoate.

### Step 3: Preparation of 3-[4-[3-(trifluoromethyl)-1,2,4-thiadiazol-5-yl]pyridin-1-ium-1-yl]propanoic acid acetate A48

To a solution of ethyl 3-[4-[3-(trifluoromethyl)-1,2,4-thiadiazol-5-yl]-3,6-dihydro-2H-pyridin-1-yl]propanoate (0.2 g) in 1,4-dioxane (4.78 mL) was added 1-bromopyrrolidine-2,5-dione (0.19 g). The reaction mixture was stirred at room temperature for 1 hour. To this was added cyclohexane (20 mL) and the mixture was stirred for 10 minutes. The solvent was decanted from a residue, which was washed with ethyl acetate. The residue was purified by preparative reverse phase HPLC to give the ester. The ester was dissolved in 1:1 acetic acid and water and heated at 80° C. for 18 hours. The reaction mixture was concentrated and the residue triturated with acetone to give 3-[4-[3-(trifluoromethyl)-1,2,4-thiadiazol-5-yl]pyridin-1-ium-1-yl]propanoic acid acetate.

¹H NMR (400 MHz, D₂O) 9.20 (d, 2H), 8.67 (d, 2H), 4.98 (t, 2H), 3.19 (t, 2H), 2.02 (s, 3H) (CO₂H proton missing)

Also isolated from this reaction and hydrolysed in a similar way was 3-[3-bromo-4-[3-(trifluoromethyl)-1,2,4-thiadiazol-5-yl]pyridin-1-ium-1-yl]propanoic acid acetate A79

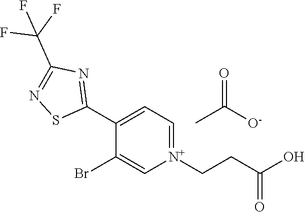

¹H NMR (400 MHz, D₂O) 9.68 (s, 1H), 9.21 (dd, 1H), 8.96 (d, 1H), 4.99 (t, 2H), 3.25 (t, 2H), 2.04 (s, 3H) (CO₂H proton missing)

### Example 16: Preparation of 3-[4-(3-hydroxy-1,2,4-thiadiazol-5-yl)pyridin-1-ium-1-yl]propanoic acid bromide A97

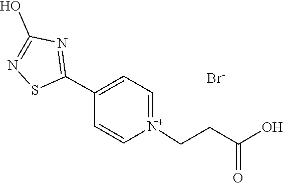 [SMILES: O=C(O)CC[n+]1ccc(-c2nc(O)ns2)cc1 ; [Br-]]

### Step 1: Preparation of 5-(4-pyridyl)-1,2,4-thiadiazol-3-ol

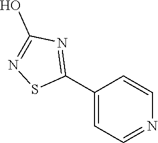 [SMILES: Oc1nsc(-c2ccncc2)n1]

To a solution of 3-bromo-5-(4-pyridyl)-1,2,4-thiadiazole (0.25 g) in N,N-dimethylformamide (1.9 mL) was added cesium carbonate (0.673 g) followed by (E)-benzaldehyde oxime (0.126 g). The reaction mixture was heated at 40° C. overnight. It was then heated at 80° C. for 30 hours. The reaction was concentrated and the residue was washed with tert-butyl methyl ether (×3) and dried to give crude 5-(4-pyridyl)-1,2,4-thiadiazol-3-ol, which was used without further purification.

¹H NMR (400 MHz, DMSO-d6) 8.63-8.67 (m, 2H), 7.64-7.67 (m, 2H) (OH proton missing)

### Step 2: Preparation of [5-(4-pyridyl)-1,2,4-thiadiazol-3-yl]-2,2-dimethylpropanoate

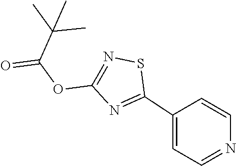 [SMILES: CC(C)(C)C(=O)Oc1nsc(-c2ccncc2)n1]

A solution of 5-(4-pyridyl)-1,2,4-thiadiazol-3-ol (0.05 g) in dichloromethane (2.5 mL) was cooled to ˜0° C. then triethylamine (0.024 mL) and 2,2-dimethylpropanoyl chloride (0.021 mL) were added. The reaction was stirred at ˜0° C. for 2 hours. The reaction mixture was partitioned between water and dichloromethane. The aqueous layer was extracted with further dichloromethane (×2). The combined organic phases were dried over sodium sulfate and concentrated to give [5-(4-pyridyl)-1,2,4-thiadiazol-3-yl]-2,2-dimethylpropanoate, which was used without further purification.

### Step 3: Preparation of 3-[4-(3-hydroxy-1,2,4-thiadiazol-5-yl)pyridin-1-ium-1-yl]propanoic acid bromide A97

A mixture of [5-(4-pyridyl)-1,2,4-thiadiazol-3-yl]-2,2-dimethylpropanoate (0.5 g), acetonitrile (10 mL) and 3-bromopropanoic acid (0.36 g) was heated at 60° C. for 12 hours. The reaction was concentrated and the residue washed with tert-butyl methyl ether and dried to give 3-[4-(3-hydroxy-1,2,4-thiadiazol-5-yl)pyridin-1-ium-1-yl]propanoic acid bromide.

¹H NMR (400 MHz, D₂O) 9.07 (d, 2H), 8.45 (d, 2H), 4.90 (t, 2H), 3.18 (t, 2H) (CO₂H and OH protons missing)

### Example 17: Preparation of 3-[3-methylsulfonyl-4-(1,2,4-thiadiazol-5-yl)pyridin-1-ium-1-yl]propanoic acid bromide A93

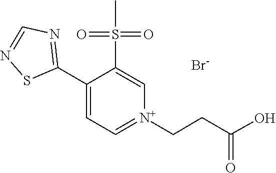 [SMILES: CS(=O)(=O)c1c[n+](CCC(=O)O)ccc1-c1ncns1 ; [Br-]]

### Step 1: Preparation of 3-methylsulfonylpyridine-4-carbonitrile

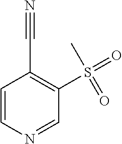

To a solution of 3-chloropyridine-4-carbonitrile (0.1 g) in N,N-dimethylformamide (1 mL) was added sodium methanesulfinate (0.174 g) and the mixture was heated at 140° C. for 4 hours. The reaction mixture was cooled to room temperature and partitioned between ethyl acetate (20 mL) and water (10 mL). The aqueous layer was extracted with further ethyl acetate (2×20 mL). The combined organic layers were dried over sodium sulfate, concentrated and purified by silica gel chromatography eluting with 0 to 50% ethyl acetate in cyclohexane to give 3-methylsulfonylpyridine-4-carbonitrile as a yellow solid.

¹H NMR (400 MHz, CDCl₃) 9.30 (br s, 1H), 9.02 (br s, 1H), 7.75 (br s, 1H), 3.25 ppm (br s, 3H)

### Step 2: Preparation of 3-methylsulfonylpyridine-4-carbothioamide

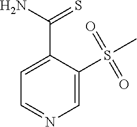

To a solution of 3-methylsulfonylpyridine-4-carbonitrile (1.7 g) in pyridine (1.7 mL) was added triethylamine (1.2 mL) and ammonium polysulfide (48%, 3.4 mL) and the mixture was heated at 60° C. for 2 hours. The reaction mixture was cooled to room temperature and partitioned between ethyl acetate (120 mL) and water (30 mL). The aqueous layer was extracted with further ethyl acetate (2×100 mL). The combined organic layers were dried over sodium sulfate and concentrated. The residue was triturated with tert-butyl methyl ether (30 mL) to give 3-methylsulfonylpyridine-4-carbothioamide as a light yellow solid.

¹H NMR (400 MHz, DMSO-d₆) 10.47 (br s, 1H), 10.09 (br s, 1H), 8.99 (s, 1H), 8.85 (d, 1H), 7.39 (d, 1H), 3.47 ppm (s, 3H)

### Step 3: Preparation of N-(dimethylaminomethylene)-3-methylsulfonyl-pyridine-4-carbothioamide

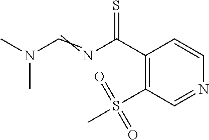 [SMILES: CN(C)C=NC(=S)c1ccncc1S(C)(=O)=O]

A mixture of 3-methylsulfonylpyridine-4-carbothioamide (1.45 g) and 1,1-dimethoxy-N,N-dimethyl-methanamine (7.25 mL) was stirred at room temperature for 3 hours. The reaction mixture was concentrated and the residue was triturated with tert-butyl methyl ether (40 mL) and dried to give N-(dimethylaminomethylene)-3-methylsulfonyl-pyridine-4-carbothioamide, which was used without further purification.

### Step 4: Preparation of 5-(3-methylsulfonyl-4-pyridyl)-1,2,4-thiadiazole

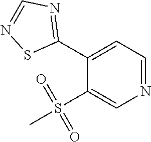

To a mixture of N-(dimethylaminomethylene)-3-methylsulfonyl-pyridine-4-carbothioamide (1.52 g), pyridine (0.906 mL) and methanol (15.2 mL), at room temperature, was added a solution of amino hydrogen sulfate (0.697 g) in methanol (7.6 mL). The reaction mixture was stirred at room temperature for 1 hour. The reaction mixture was quenched with sat. aqueous sodium bicarbonate solution and extracted with ethyl acetate (20 mL). The organic layer was dried over sodium sulfate, concentrated and purified by silica gel chromatography eluting with a mixture of ethyl acetate in cyclohexane to give 5-(3-methylsulfonyl-4-pyridyl)-1,2,4-thiadiazole.

¹H NMR (400 MHz, CDCl₃) 9.45 (s, 1H), 9.04 (d, 1H), 8.84 (s, 1H), 7.62 (d, 1H), 3.46 (s, 3H)

### Step 5: Preparation of 3-[3-methylsulfonyl-4-(1,2,4-thiadiazol-5-yl)pyridin-1-ium-1-yl]propanoic acid bromide A93

A mixture of 5-(3-methylsulfonyl-4-pyridyl)-1,2,4-thiadiazole (0.2 g) and 3-bromopropanoic acid (0.14 g) in acetonitrile (2 mL) was heated at 80° C. for 40 hours. The reaction mixture was concentrated and the residue was triturated with tert-butyl methyl ether (40 mL) and dried to give 3-[3-methylsulfonyl-4-(1,2,4-thiadiazol-5-yl)pyridin-1-ium-1-yl]propanoic acid bromide.

¹H NMR (400 MHz, D₂O) 9.85 (s, 1H), 9.50 (d, 1H), 9.07 (s, 1H), 8.60 (d, 1H), 5.13 (t, 2H), 3.67 (s, 3H), 3.30 (t, 2H) (CO₂H proton missing)

### Example 18: Preparation of 2-[4-(5-phenyl-1,3,4-oxadiazol-2-yl)pyridin-1-ium-1-yl]ethanol 2,2,2-trifluoroacetate A69

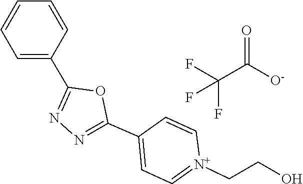 [SMILES: OCC[n+]1ccc(-c2nnc(-c3ccccc3)o2)cc1 ; O=C([O-])C(F)(F)F]

A mixture of 2-phenyl-5-(4-pyridyl)-1,3,4-oxadiazole (0.1 g), 1,2-dichloroethane (6 mL) and 1,3,2-dioxathiolane 2,2-dioxide (0.064 g) was heated at 85° C. overnight. The resulting precipitate is filtered off and the filtrate was concentrated and purified by preparative reverse phase HPLC (trifluoroacetic acid was present in the eluent) to afford 2-[4-(5-phenyl-1,3,4-oxadiazol-2-yl)pyridin-1-ium-1-yl]ethanol 2,2,2-trifluoroacetate.

¹H NMR (400 MHz, DMSO-d₆) 9.16-9.33 (m, 2H), 8.75-8.88 (m, 2H), 8.17-8.32 (m, 2H), 7.55-7.80 (m, 3H), 4.68-4.83 (m, 2H), 3.82-4.00 (m, 2H) (OH proton missing)

Additional compounds in Table A (below) were prepared by analogues procedures, from appropriate starting materials. The skilled person would understand that the compounds of formula (I) may exist as an agronomically acceptable salt, a zwitterion or an agronomically acceptable salt of a zwitterion as described hereinbefore. Where mentioned the specific counterion is not considered to be limiting, and the compound of formula (I) may be formed with any suitable counter ion.

NMR spectra contained herein were recorded on either a 400 MHz Bruker AVANCE III HD equipped with a Bruker SMART probe unless otherwise stated. Chemical shifts are expressed as ppm downfield from TMS, with an internal reference of either TMS or the residual solvent signals. The following multiplicities are used to describe the peaks: s=singlet, d=doublet, t=triplet, dd=double doublet, dt=double triplet, q=quartet, quin=quintet, m=multiplet. Additionally br. is used to describe a broad signal and app. is used to describe and apparent multiplicity.

TABLE A
Physical Data for Compounds of the Invention

| Compound | | |
|---|---|---|
| Number | Structure | ¹H NMR |
| A1 | 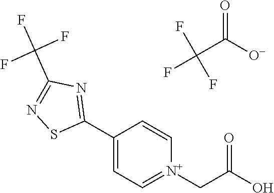 [SMILES: O=C(O)C[n+]1ccc(-c2nc(C(F)(F)F)ns2)cc1 ; O=C([O-])C(F)(F)F] | (400 MHz, D₂O) 9.06 (d, 2H), 8.69 (d, 2H), 5.41 (s, 2H) (CO₂H proton missing) |
| A2 | 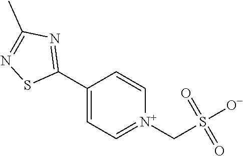 [SMILES: Cc1nsc(-c2cc[n+](CS(=O)(=O)[O-])cc2)n1] | (400 MHz, D₂O) 9.02-9.20 (m, 2H) 8.55-8.68 (m, 2H) 5.58-5.81 (m, 2H) 2.65-2.82 (m, 3H) |
| A3 | 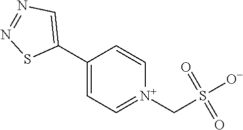 [SMILES: O=S(=O)([O-])C[n+]1ccc(-c2cnns2)cc1] | (400 MHz, D₂O) 9.41-9.49 (m, 1H) 8.95-9.04 (m, 2H) 8.37-8.49 (m, 2H) 5.68 (br s, 2H) |
| A4 | 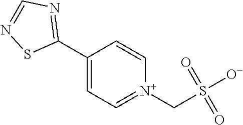 [SMILES: O=S(=O)([O-])C[n+]1ccc(-c2ncns2)cc1] | (400 MHz, D₂O) 9.25-9.02 (m, 3H), 8.81-8.66 (m, 2H), 5.85-5.74 (m, 2H) |
| A5 | 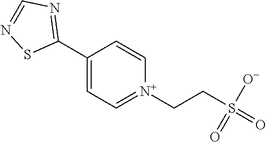 [SMILES: O=S(=O)([O-])CC[n+]1ccc(-c2ncns2)cc1] | (400 MHz, D₂O) 9.02-9.11 (m, 2H) 8.87-8.99 (m, 1H) 8.47-8.60 (m, 2H) 4.94-5.05 (m, 2H) 3.48-3.61 (m, 2H) |
| A6 | 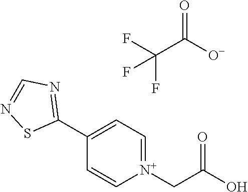 [SMILES: O=C(O)C[n+]1ccc(-c2ncns2)cc1 ; O=C([O-])C(F)(F)F] | (400 MHz, D₂O) 8.96-8.88 (m, 3H), 8.58-8.54 (m, 2H), 5.40 (s, 2H) |
| A7 | 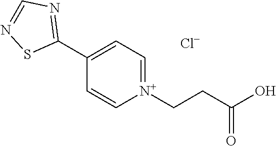 [SMILES: O=C(O)CC[n+]1ccc(-c2ncns2)cc1 ; [Cl-]] | (400 MHz, D₂O) 9.05 (d, 2H), 8.92 (s, 1H), 8.51 (d, 2H), 4.87 (t, 2H), 3.15 (t, 2H) (CO₂H proton missing) |
| A8 | 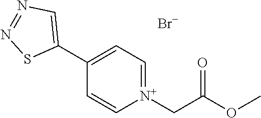 [SMILES: COC(=O)C[n+]1ccc(-c2cnns2)cc1 ; [Br-]] | (400 MHz, D₂O) 9.39-9.45 (m, 1H) 8.85-8.93 (m, 2H) 8.35-8.44 (m, 2H) 5.55 (s, 2H) 3.73-3.84 (m, 3H) |
| A9 | 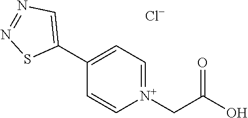 [SMILES: O=C(O)C[n+]1ccc(-c2cnns2)cc1 ; [Cl-]] | (400 MHz, D₂O) 9.38-9.43 (m, 1H) 8.83-8.90 (m, 2H) 8.33-8.40 (m, 2H) 5.40 (s, 2H) (CO₂H proton missing) |
| A10 | 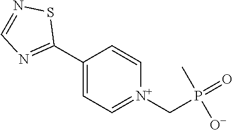 [SMILES: CP(=O)([O-])C[n+]1ccc(-c2ncns2)cc1] | (400 MHz, CD₃OD) 9.08 (s, 3H), 8.77 (br d, 2H), 5.12 (br s, 2H), 1.71-1.46 (m, 3H) |
| A11 | 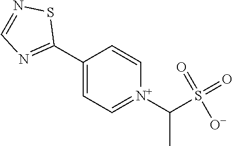 [SMILES: CC([n+]1ccc(-c2ncns2)cc1)S(=O)(=O)[O-]] | (400 MHz, D₂O) 9.13 (d, 2H), 8.98 (s, 1H), 8.62 (d, 2H), 5.86 (d, 1H), 2.02 (d, 3H) |
| A12 | 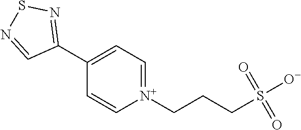 [SMILES: O=S(=O)([O-])CCC[n+]1ccc(-c2cnsn2)cc1] | (400 MHz, D₂O) 9.41 (s, 1H), 9.01 (d, 2H), 8.66 (d, 2H), 4.72-4.79 (m, 2H), 2.99-3.02 (m, 2H), 2.47-2.53 (m, 2H) |
| A13 | 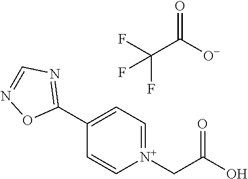 [SMILES: O=C(O)C[n+]1ccc(-c2ncno2)cc1 ; O=C([O-])C(F)(F)F] | (400 MHz, D₂O) 9.06-9.00 (m, 2H), 8.91 (s, 1H), 8.68 (d, 2H), 5.48 (s, 2H) (CO₂H Proton missing) |
| A14 | 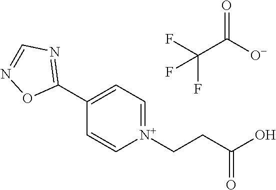 [SMILES: O=C(O)CC[n+]1ccc(-c2ncno2)cc1 ; O=C([O-])C(F)(F)F] | (400 MHz, D₂O) 9.08-9.19 (m, 2H), 8.86-8.96 (m, 1H), 8.64 (d, 2H), 4.85- 4.96 (m, 2H), 3.15 (t, 2H) (CO₂H proton missing) |
| A15 | 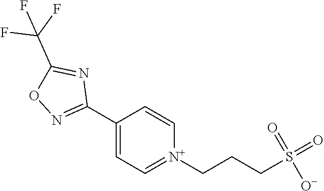 [SMILES: O=S(=O)([O-])CCC[n+]1ccc(-c2noc(C(F)(F)F)n2)cc1] | (400 MHz, D₂O) 9.09 (d, 2H), 8.65 (br d, 2H), 4.78-4.84 (m, 2H), 2.95 (t, 2H), 2.44 (br t, 2H) |
| A16 | 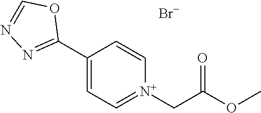 [SMILES: COC(=O)C[n+]1ccc(-c2nnco2)cc1 ; [Br-]] | (400 MHz, D₂O) 9.17 (s, 1 H), 9.03 (d, 2H), 8.66 (d, 2H), 5.62 (s, 2H), 3.79 (s, 3H) |
| A17 | 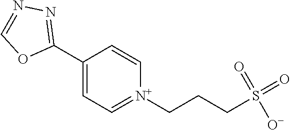 [SMILES: O=S(=O)([O-])CCC[n+]1ccc(-c2nnco2)cc1] | (400 MHz, D₂O) 9.15 (d, 1H), 9.09 (d, 2H), 8.61 (br d, 2H), 4.80 (t, 2H), 2.90- 2.98 (m, 2H), 2.39-2.49 (m, 2H) |
| A18 | 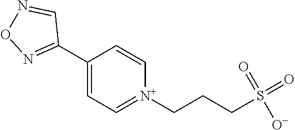 [SMILES: O=S(=O)([O-])CCC[n+]1ccc(-c2cnon2)cc1] | (400 MHz, D₂O) 8.89 (br d, 2H), 8.30 (br d, 2H), 2.91 (t, 2H), 2.45-2.34 (m, 2H) (one CH₂ underwater peak, oxadiazole proton exchanged) |
| A19 | 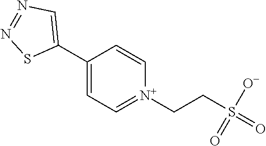 [SMILES: O=S(=O)([O-])CC[n+]1ccc(-c2cnns2)cc1] | (400 MHz, D₂O) 9.35-9.41 (m, 1H), 8.89-8.97 (m, 2H), 8.32 (d, 2H), 4.89- 5.01 (m, 2H), 3.52 (s, 1H), 3.55 (d, 1H) |
| A20 | 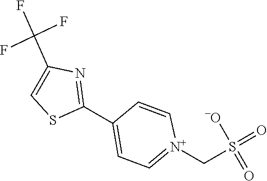 | (400 MHz, D₂O) 9.08 (d, 2H), 8.66 (d, 2H), 8.63 (s, 1H), 5.75 (s, 2H) |
| A21 | 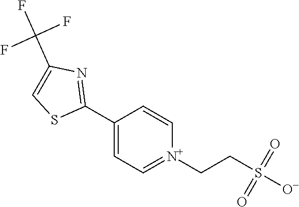 [SMILES: O=S(=O)([O-])CC[n+]1ccc(-c2nc(C(F)(F)F)cs2)cc1] | (400 MHz, D₂O) 9.06 (d, 2H), 8.54- 8.62 (m, 3H), 5.07 (t, 2H), 3.65 (t, 2H) |
| A22 | 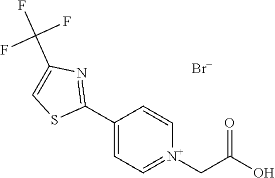 [SMILES: O=C(O)C[n+]1ccc(-c2nc(C(F)(F)F)cs2)cc1 ; [Br-]] | (400 MHz, D₂O) 8.95 (d, 2H), 8.62 (t, 3H), 5.40 (s, 2H) (CO₂H proton missing) |
| A23 | 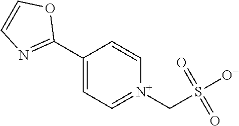 [SMILES: O=S(=O)([O-])C[n+]1ccc(-c2ncco2)cc1] | (400 MHz, D₂O) 8.98 (d, 2H), 8.54 (d, 2H), 8.18 (d, 1H), 7.54 (d, 1H), 5.65 (s, 2H) |
| A24 | 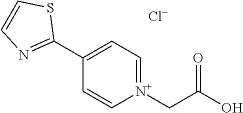 [SMILES: O=C(O)C[n+]1ccc(-c2nccs2)cc1 ; [Cl-]] | (400 MHz, D₂O) 8.83 (d, 2H), 8.51 (d, 2H), 8.17 (d, 1H), 8.04 (d, 1H), 5.33 (s, 2H) (CO₂H proton missing) |
| A25 | 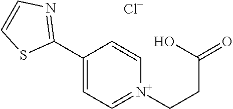 [SMILES: O=C(O)CC[n+]1ccc(-c2nccs2)cc1 ; [Cl-]] | (400 MHz, D₂O) 8.95 (d, 2H), 8.46 (d, 2H), 8.14 (d, 1H), 8.01 (d, 1H), 4.86 (t, 2H), 3.18 (t, 2H) (CO₂H proton missing) |
| A26 | 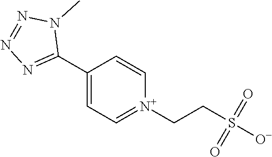 [SMILES: Cn1nnnc1-c1cc[n+](CCS(=O)(=O)[O-])cc1] | (400 MHz, D₂O) 9.04-9.21 (m, 2H), 8.43-8.55 (m, 2H), 5.00-5.13 (m, 2H), 4.23-4.35 (m, 3H), 3.49-3.68 (m, 2H) |
| A27 | 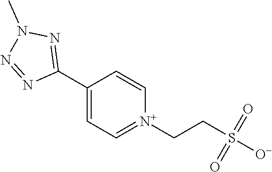 [SMILES: Cn1nnc(-c2cc[n+](CCS(=O)(=O)[O-])cc2)n1] | (400 MHz, D₂O) 8.97-9.10 (m, 2H), 8.54-8.66 (m, 2H), 4.95-5.06 (m, 2H), 4.41-4.53 (m, 3H), 3.50-3.63 (m, 2H) |
| A28 | 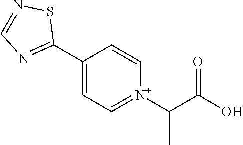 [SMILES: CC(C(=O)O)[n+]1ccc(-c2ncns2)cc1] | (400 MHz, D₂O) 9.03-8.90 (m, 3H), 8.52 (d, 2H), 5.39-5.32 (m, 1H), 1.84 (d, 3H) (CO₂H proton missing) |
| A29 | 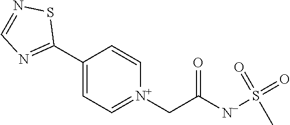 [SMILES: CS(=O)(=O)[N-]C(=O)C[n+]1ccc(-c2ncns2)cc1] | (400 MHz, D₂O) 8.96 (s, 1H), 8.92 (d, 2H), 8.59 (d, 2H), 5.52 (s, 2H), 3.17 (s, 3H) |
| A30 | 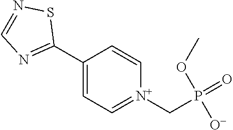 [SMILES: COP(=O)([O-])C[n+]1ccc(-c2ncns2)cc1] | (400 MHz, D₂O) 8.96-8.88 (m, 3H), 8.54 (d, 2H), 4.90-4.83 (m, 2H), 3.54 (d, 3H) |
| A31 | 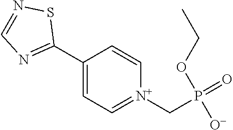 [SMILES: CCOP(=O)([O-])C[n+]1ccc(-c2ncns2)cc1] | (400 MHz, D₂O) 8.95-8.87 (m, 3H), 8.56-8.51 (m, 2H), 4.86-4.79 (m, 2H), 3.90-3.82 (m, 2H), 1.12 (t, 3H) |
| A32 | 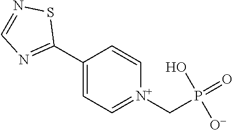 [SMILES: O=P([O-])(O)C[n+]1ccc(-c2ncns2)cc1] | (400 MHz, D₂O) 9.01-8.86 (m, 3H), 8.54 (d, 2H), 4.84-4.78 (m, 2H) (POH proton missing) |
| A33 | 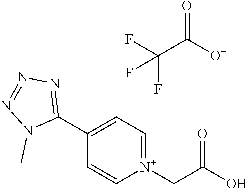 [SMILES: Cn1nnnc1-c1cc[n+](CC(=O)O)cc1 ; O=C([O-])C(F)(F)F] | (400 MHz, D₂O) 9.03 (d, 2H), 8.50 (d, 2H), 5.44 (s, 2H), 4.28 (s, 3H) (CO₂H proton missing) |
| A34 | 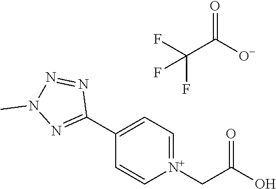 [SMILES: Cn1nnc(-c2cc[n+](CC(=O)O)cc2)n1 ; O=C([O-])C(F)(F)F] | (400 MHz, D₂O) 8.91-8.85 (m, 2H), 8.63-8.59 (m, 2H), 5.35 (s, 2H), 4.44 (s, 3H) (CO₂H proton missing) |
| A35 | 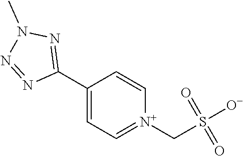 [SMILES: Cn1nnc(-c2cc[n+](CS(=O)(=O)[O-])cc2)n1] | (400 MHz, D₂O) 9.13-9.05 (m, 2H), 8.78-8.67 (m, 2H), 5.77-5.70 (m, 2H), 4.56-4.48 (m, 3H) |
| A36 | 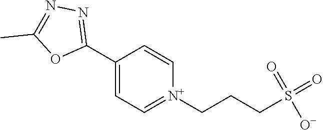 [SMILES: Cc1nnc(-c2cc[n+](CCCS(=O)(=O)[O-])cc2)o1] | (400 MHz, D₂O) 9.05 (d, 2H), 8.52 (d, 2H), 4.78 (t, 2H), 2.94 (t, 2H), 2.63 (s, 3H), 2.43 (t, 2H) |
| A37 | 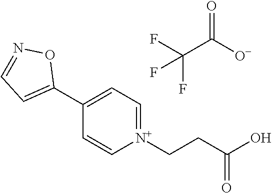 [SMILES: O=C(O)CC[n+]1ccc(-c2ccno2)cc1 ; O=C([O-])C(F)(F)F] | (400 MHz, D₂O) 9.02 (d, 2H), 8.67 (s, 1H), 8.43 (d, 2H), 7.39 (d, 1H), 4.90 (t, 2H), 3.20 (t, 2H) (CO₂H proton missing) |
| A38 | 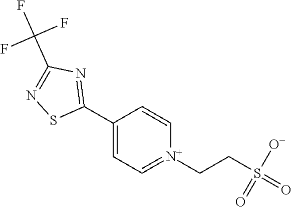 [SMILES: O=S(=O)([O-])CC[n+]1ccc(-c2nc(C(F)(F)F)ns2)cc1] | (400 MHz, D₂O) 9.14 (d, 2H), 8.63 (d, 2H), 5.06 (t, 2H), 3.60 (t, 2H) |
| A39 | 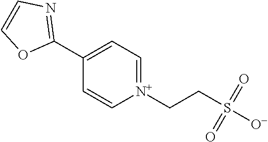 [SMILES: O=S(=O)([O-])CC[n+]1ccc(-c2ncco2)cc1] | (400 MHz, D₂O) 8.97 (d, 2H), 8.47 (d, 2H), 8.14 (d, 1H), 7.50 (d, 1H), 3.55 (t, 2H) 4.97 (t, 2H) |
| A40 | 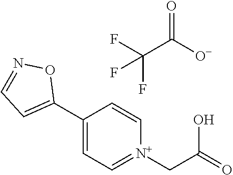 [SMILES: O=C(O)C[n+]1ccc(-c2ccno2)cc1 ; O=C([O-])C(F)(F)F] | (400 MHz, D₂O) 8.89 (d, 2H), 8.67 (d, 1H), 8.46 (d, 2H), 7.42 (d, 1H), 5.37 (s, 2H) (CO₂H proton missing) |
| A41 | 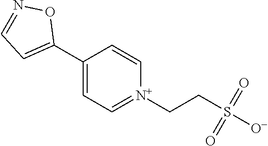 [SMILES: O=S(=O)([O-])CC[n+]1ccc(-c2ccno2)cc1] | (400 MHz, D₂O) 9.03 (d, 2H), 8.67 (d, 1H), 8.45 (d, 2H), 7.41 (d, 1H), 5.01- 5.07 (m, 2H), 3.60-3.64 (m, 2H) |
| A42 | 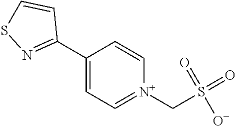 [SMILES: O=S(=O)([O-])C[n+]1ccc(-c2ccsn2)cc1] | (400 MHz, DMSO-d₆) 9.39 (d, 1H), 9.08 (d, 2H), 8.72 (d, 2H), 8.37 (d, 1H), 5.45 (s, 2H) |
| A43 | 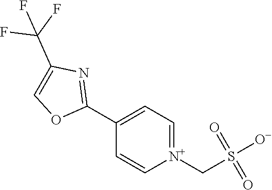 [SMILES: O=S(=O)([O-])C[n+]1ccc(-c2nc(C(F)(F)F)co2)cc1] | (400 MHz, D₂O) 9.13 (d, 2H), 8.75 (d, 1H), 8.66 (d, 2H), 5.75 (s, 2H) |
| A44 | 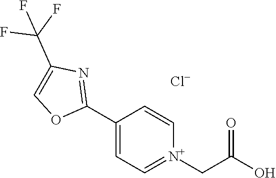 [SMILES: O=C(O)C[n+]1ccc(-c2nc(C(F)(F)F)co2)cc1 ; [Cl-]] | (400 MHz, D₂O) 8.97 (d, 2H), 8.70- 8.76 (m, 1H), 8.60 (d, 2H), 5.38 (s, 2H) (CO₂H proton missing) |
| A45 | 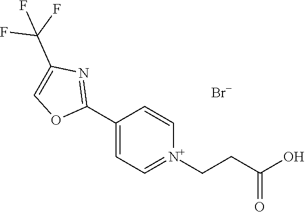 [SMILES: O=C(O)CC[n+]1ccc(-c2nc(C(F)(F)F)co2)cc1 ; [Br-]] | (400 MHz, D₂O) 9.11 (d, 2H), 8.65- 8.79 (m, 1H), 8.56 (d, 2H), 4.93 (t, 2H) 3.20 (t, 2H) (CO₂H proton missing) |
| A46 | 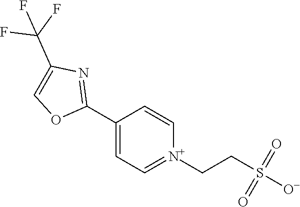 [SMILES: O=S(=O)([O-])CC[n+]1ccc(-c2nc(C(F)(F)F)co2)cc1] | (400 MHz, D₂O) 9.09 (d, 2H), 8.71 (d, 1H), 8.57 (d, 2H), 5.11-5.02 (m, 2H), 3.56-3.66 (m, 2H) |
| A47 | 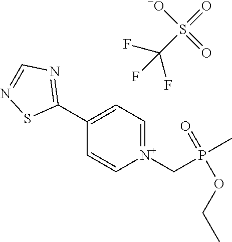 | (400 MHz, DMSO-d₆) 9.33 (s, 1H), 9.16 (d, 2H), 8.85 (d, 2H), 5.37 (d, 2H), 4.14-4.03 (m, 2H), 1.69 (d, 3H), 1.27- 1.19 (m, 3H) |
| A48 | 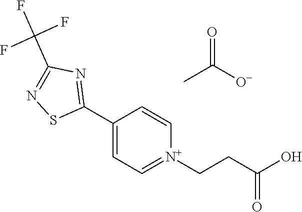 | (400 MHz, D₂O) 9.20 (d, 2H), 8.67 (d, 2H), 4.98 (t, 2H), 3.19 (t, 2H), 2.02 (s, 3H) (CO₂H proton missing) |
| A49 | 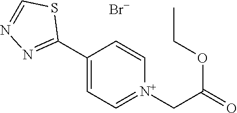 [SMILES: CCOC(=O)C[n+]1ccc(-c2nncs2)cc1 ; [Br-]] | (400 MHz, DMSO-d₆) 9.71 (s, 1H), 9.25 (d, 2H), 8.81 (d, 2H), 5.75 (s, 2H), 4.27 (q, 2H), 1.27 (t, 3H) |
| A50 | 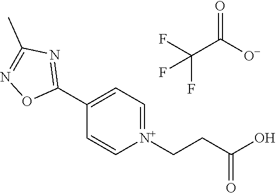 [SMILES: Cc1noc(-c2cc[n+](CCC(=O)O)cc2)n1 ; O=C([O-])C(F)(F)F] | (400 MHz, D₂O) 9.14 (d, 2H), 8.58 (d, 2H), 4.91 (t, 2H), 3.16 (t, 2H), 2.45 (s, 3H) (CO₂H proton missing) |
| A51 | 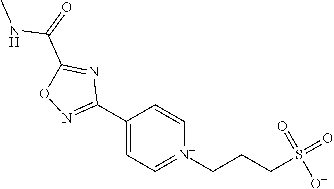 [SMILES: CNC(=O)c1nc(-c2cc[n+](CCCS(=O)(=O)[O-])cc2)no1] | (400 MHz, D₂O) 9.06 (d, 2H), 8.64 (d, 2H), 4.80 (t, 2H), 2.97-2.90 (m, 5H), 2.49-2.39 (m, 2H) (NH proton missing) |
| A52 | 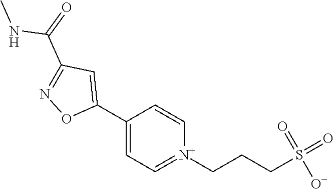 [SMILES: CNC(=O)c1cc(-c2cc[n+](CCCS(=O)(=O)[O-])cc2)on1] | (400 MHz, D₂O) 8.88-9.03 (m, 2H), 8.36-8.48 (m, 2H), 7.53-7.66 (m, 1H), 4.64-4.82 (m, 2H), 2.93 (t, 2H), 2.87 (s, 3H), 2.42 (quin, 2H) (NH proton missing) |
| A53 | 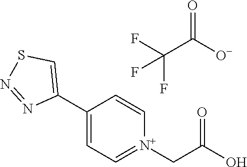 [SMILES: O=C(O)C[n+]1ccc(-c2csnn2)cc1 ; O=C([O-])C(F)(F)F] | (400 MHz, D₂O) 9.80 (s, 1H), 8.84 (d, 2H), 8.62 (d, 2H), 5.39 (s, 2H) (CO₂H proton missing) |
| A54 | 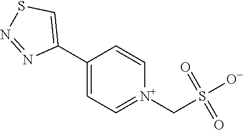 [SMILES: O=S(=O)([O-])C[n+]1ccc(-c2csnn2)cc1] | (400 MHz, DMSO-d₆) 10.32 (s, 1H), 9.09-9.20 (m, 2H), 8.88 (d, 2H), 5.43- 5.52 (m, 2H) |
| A55 | 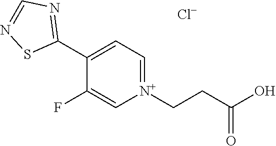 [SMILES: O=C(O)CC[n+]1ccc(-c2ncns2)c(F)c1 ; [Cl-]] | (400 MHz, D₂O) 9.34-9.45 (m, 1H), 8.94-9.10 (m, 2H), 8.74-8.83 (m, 1H), 4.88-5.03 (m, 2H), 3.11-3.25 (m, 2H) (CO₂H proton missing) |
| A56 | 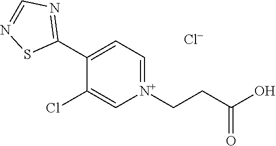 [SMILES: O=C(O)CC[n+]1ccc(-c2ncns2)c(Cl)c1 ; [Cl-]] | (400 MHz, D₂O) 9.40-9.52 (m, 1H), 8.98-9.11 (m, 2H), 8.82-8.92 (m, 1H), 4.87-4.96 (m, 2H), 3.08-3.26 (m, 2H) (CO₂H proton missing) |
| A57 | 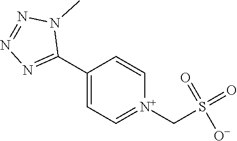 [SMILES: Cn1nnnc1-c1cc[n+](CS(=O)(=O)[O-])cc1] | (400 MHz, DMSO-d₆) 9.34-9.15 (m, 2H), 8.78-8.60 (m, 2H), 5.59 (br s, 2H), 4.37 (br s, 3H) |
| A58 | 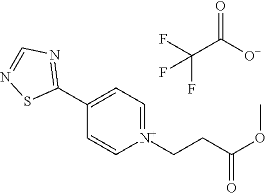 [SMILES: COC(=O)CC[n+]1ccc(-c2ncns2)cc1 ; O=C([O-])C(F)(F)F] | (400 MHz, D₂O) 9.07 (d, 2H), 8.94 (s, 1H), 8.54 (d, 2H), 4.90 (t, 2H), 3.60 (s, 3H), 3.18 (t, 2H) |
| A59 | 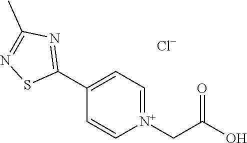 [SMILES: Cc1nsc(-c2cc[n+](CC(=O)O)cc2)n1 ; [Cl-]] | (400 MHz, D₂O) 8.93-8.89 (m, 2H), 8.51-8.46 (m, 2H), 5.37 (s, 2H), 2.67 (s, 3H) (CO₂H proton missing) |
| A60 | 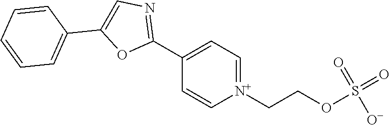 [SMILES: O=S(=O)([O-])OCC[n+]1ccc(-c2ncc(-c3ccccc3)o2)cc1] | (400 MHz, DMSO-d₆) 9.18-9.10 (m, 2H), 8.69 (d, 2H), 8.21 (s, 1H), 8.01 (d, 2H), 7.61-7.47 (m, 3H), 4.91-4.82 (m, 2H), 4.34-4.21 (m, 2H) |
| A61 | 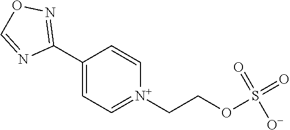 [SMILES: O=S(=O)([O-])OCC[n+]1ccc(-c2ncon2)cc1] | (400 MHz, DMSO-d₆) 10.02 (s. 1H), 9.28-9.19 (m, 2H), 8.72 (br d, 2H), 4.99-4.88 (m, 2H), 4.28 (br s, 2H) |
| A62 | 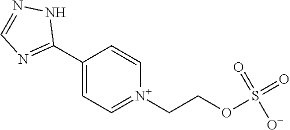 [SMILES: O=S(=O)([O-])OCC[n+]1ccc(-c2ncn[nH]2)cc1] | (400 MHz, DMSO-d₆) 14.94 (br s, 1H), 9.07 (d, 2H), 8.93 (s, 1H), 8.60 (d, 2H), 4.90-4.77 (m, 2H), 4.30-4.19 (m, 2H) |
| A63 | 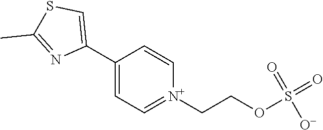 [SMILES: Cc1nc(-c2cc[n+](CCOS(=O)(=O)[O-])cc2)cs1] | (400 MHz, DMSO-d₆) 8.94-9.08 (m, 2H), 8.79-8.90 (m, 1H), 8.47-8.61 (m, 2H), 4.69-4.85 (m, 2H), 4.14- 4.31 (m, 2H), 2.69-2.84 (m, 3H) |
| A64 | 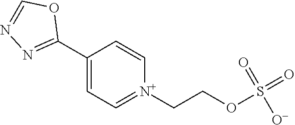 [SMILES: O=S(=O)([O-])OCC[n+]1ccc(-c2nnco2)cc1] | (400 MHz, DMSO-d₆) 9.60-9.79 (m, 1H), 9.17-9.31 (m, 2H), 8.64-8.81 (m, 2H), 4.83-5.02 (m, 2H), 4.19- 4.36 (m, 2H) |
| A65 | 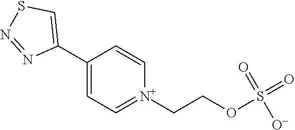 [SMILES: O=S(=O)([O-])OCC[n+]1ccc(-c2csnn2)cc1] | (400 MHz, DMSO-d₆) 10.23-10.36 (m, 1H), 9.09-9.24 (m, 2H), 8.73- 8.96 (m, 2H), 4.73-4.94 (m, 2H), 4.18- 4.34 (m, 2H) |
| A66 | 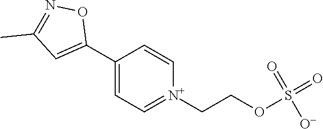 [SMILES: Cc1cc(-c2cc[n+](CCOS(=O)(=O)[O-])cc2)on1] | (400 MHz, DMSO-d₆) 9.16 (d, 2H), 8.56 (d, 2H), 7.61-7.57 (m, 1H), 4.89- 4.78 (m, 2H), 4.32-4.19 (m, 2H), 2.39 (s, 3H) |
| A67 | 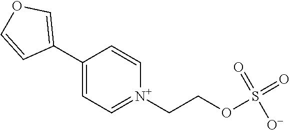 [SMILES: O=S(=O)([O-])OCC[n+]1ccc(-c2ccoc2)cc1] | (400 MHz, DMSO-d₆) 8.95 (d, 2H), 8.76-8.91 (m, 1H), 8.36 (d, 2H), 7.98 (s, 1H), 7.22-7.41 (m, 1H), 4.61-4.84 (m, 2H), 4.12-4.34 (m, 2H) |
| A68 | 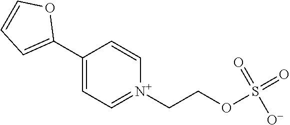 [SMILES: O=S(=O)([O-])OCC[n+]1ccc(-c2ccco2)cc1] | (400 MHz, DMSO-d₆) 8.83-9.01 (m, 2H), 8.24-8.36 (m, 2H), 8.13-8.23 (m, 1H), 7.75-7.90 (m, 1H), 6.89 (dd, 1H), 4.62-4.87 (m, 2H), 4.08-4.30 (m, 2H) |
| A69 | 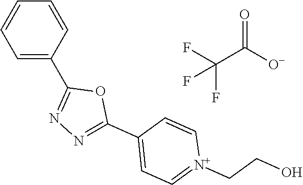 [SMILES: OCC[n+]1ccc(-c2nnc(-c3ccccc3)o2)cc1 ; O=C([O-])C(F)(F)F] | (400 MHz, DMSO-d₆) 9.16-9.33 (m, 2H), 8.75-8.88 (m, 2H), 8.17-8.32 (m, 2H), 7.55-7.80 (m, 3H), 4.68- 4.83 (m, 2H), 3.82-4.00 (m, 2H) (OH proton missing) |
| A70 | 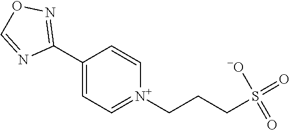 | (400 MHz, D₂O) 9.45 (s, 1H), 9.08 (d, 2H) 8.65 (d, 2H), 4.80-4.83 (m, 2H), 2.98 (t, 2H), 2.48-2.53 (m, 2H) |
| A71 | 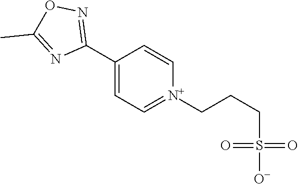 [SMILES: Cc1nc(-c2cc[n+](CCCS(=O)(=O)[O-])cc2)no1] | (400 MHz, D₂O) 9.09 (d, 2H) 8.60 (d, 2H), 4.83-4.87 (m, 2H), 3.01 (t, 2H), 2.74 (s, 3H), 2.49-2.53 (m, 2H) |
| A72 | 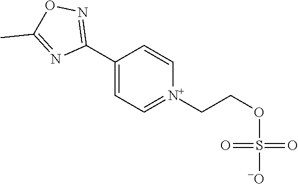 | (400 MHz, D₂O) 9.07 (d, 2H) 8.61 (d, 2H), 4.98-5.02 (m, 2H), 4.53-4.56 (m, 2H), 2.74 (s, 3H) |
| A73 | 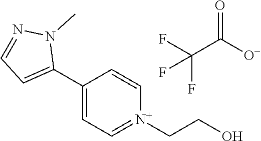 | (400 MHz, DMSO-d₆) 9.02-9.13 (m, 2H), 8.32-8.45 (m, 2H), 7.60-7.73 (m, 1H), 6.89-7.14 (m, 1H), 4.63- 4.75 (m, 2H), 4.04-4.17 (m, 2H), 3.82- 3.97 (m, 3H) (OH proton missing) |
| A74 | 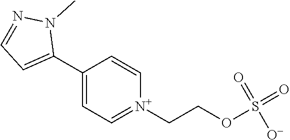 [SMILES: Cn1nccc1-c1cc[n+](CCOS(=O)(=O)[O-])cc1] | (400 MHz, DMSO-d₆) 8.99-9.16 (m, 2H), 8.30-8.45 (m, 2H), 7.61-7.70 (m, 1H), 7.02-7.13 (m, 1H), 4.78- 4.91 (m, 2H), 4.22-4.33 (m, 2H), 4.05-4.16 (m, 3H) |
| A75 | 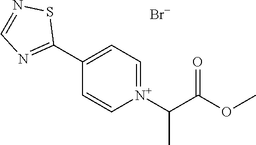 [SMILES: COC(=O)C(C)[n+]1ccc(-c2ncns2)cc1 ; [Br-]] | (400 MHz, D₂O) 9.12 (d, 2H), 9.02 (s, 1H), 8.65 (d, 2H), 5.83 (q, 1H), 3.80 (s, 3H), 1.99 (d, 3H) |
| A76 | 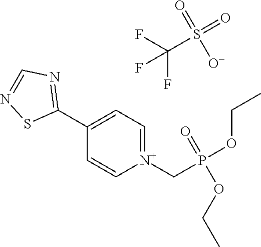 [SMILES: CCOP(=O)(C[n+]1ccc(-c2ncns2)cc1)OCC ; O=S(=O)([O-])C(F)(F)F] | (400 MHz, DMSO-d₆) 9.33 (s, 1H), 9.18 (br d, 2H), 8.87 (d, 2H), 5.54- 5.46 (m, 2H), 4.21-4.10 (m, 4H), 1.28- 1.19 (m, 6H) |
| A77 | 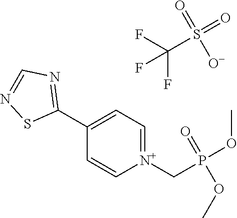 [SMILES: COP(=O)(C[n+]1ccc(-c2ncns2)cc1)OC ; O=S(=O)([O-])C(F)(F)F] | (400 MHz, DMSO-d₆) 9.33 (s, 1H), 9.22-9.17 (m, 2H), 8.88-8.83 (m, 2H), 5.54 (d, 2H), 3.83-3.76 (m, 6H) |
| A78 | 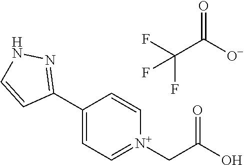 [SMILES: O=C(O)C[n+]1ccc(-c2cc[nH]n2)cc1 ; O=C([O-])C(F)(F)F] | (400 MHz, D₂O) 8.64 (d, 2H), 8.19 (d, 2H), 7.79 (d, 1H), 6.98 (d, 1H), 5.34 (s, 2H) (CO₂H and NH protons missing) |
| A79 | 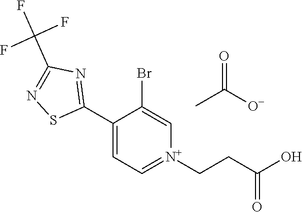 | (400 MHz, D₂O) 9.68 (s, 1H), 9.21 (dd, 1H), 8.96 (d, 1H), 4.99 (t, 2H), 3.25 (t, 2H), 2.04 (s, 3H) (CO₂H proton missing) |
| A80 | 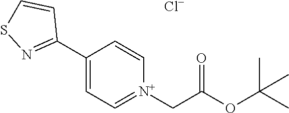 [SMILES: CC(C)(C)OC(=O)C[n+]1ccc(-c2ccsn2)cc1 ; [Cl-]] | (400 MHz, D₂O) 9.15 (d, 1H), 8.87 (d, 2H), 8.60 (d, 2H), 8.12 (d, 1H), 5.46 (s, 2H), 1.48 (s, 9H) |
| A81 | 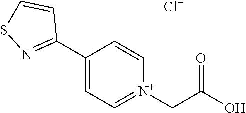 [SMILES: O=C(O)C[n+]1ccc(-c2ccsn2)cc1 ; [Cl-]] | (400 MHz, D₂O) 9.10 (d, 1H), 8.82 (d, 2H), 8.53 (d, 2H), 8.07 (d, 1H), 5.37 (s, 2H) (CO₂H proton missing) |
| A82 | 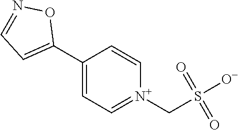 [SMILES: O=S(=O)([O-])C[n+]1ccc(-c2ccno2)cc1] | (400 MHz, D₂O) 9.02 (d, 2H), 8.66 (d, 1H), 8.50 (d, 2H), 7.43 (d, 1H), 5.69 (s, 2H) |
| A83 | 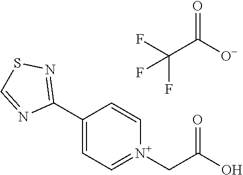 [SMILES: O=C(O)C[n+]1ccc(-c2ncsn2)cc1 ; O=C([O-])C(F)(F)F] | (400 MHz, D₂O) 10.33 (s, 1H), 8.93 (d, 2H), 8.81 (d, 2H), 5.36 (s, 2H) (CO₂H proton missing) |
| A84 | 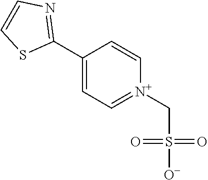 [SMILES: O=S(=O)([O-])C[n+]1ccc(-c2nccs2)cc1] | (400 MHz, D₂O) 9.02 (d, 2H), 8.62 (d, 2H), 8.25 (d, 1H), 8.13 (d, 1H), 5.73 (s, 2H) |
| A85 | 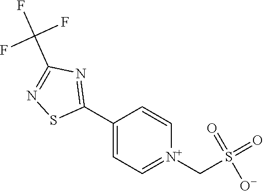 [SMILES: O=S(=O)([O-])C[n+]1ccc(-c2nc(C(F)(F)F)ns2)cc1] | (400 MHz, D₂O) 9.25 (d, 2H), 8.81 (d, 2H), 5.84 (s, 2H) |
| A86 | 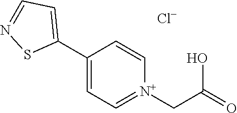 [SMILES: O=C(O)C[n+]1ccc(-c2ccns2)cc1 ; [Cl-]] | (400 MHz, D₂O) 8.87 (d, 2H), 8.68 (d, 1H), 8.36 (d, 2H), 8.05 (d, 1H), 5.44 (s, 2H) (CO₂H proton missing) |
| A87 | 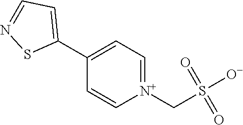 [SMILES: O=S(=O)([O-])C[n+]1ccc(-c2ccns2)cc1] | (400 MHz, D₂O) 8.99 (d, 2H), 8.71 (d, 1H), 8.42 (d, 2H), 8.10 (d, 1H), 5.71 (s, 2H) |
| A88 | 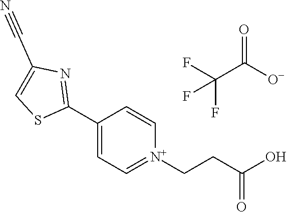 [SMILES: N#Cc1csc(-c2cc[n+](CCC(=O)O)cc2)n1 ; O=C([O-])C(F)(F)F] | (400 MHz, D₂O) 9.08 (d, 2H), 8.86 (s, 1H), 8.59 (d, 2H), 4.93 (t, 2H), 3.22 (t, 2H) (CO₂H proton missing) |
| A89 | 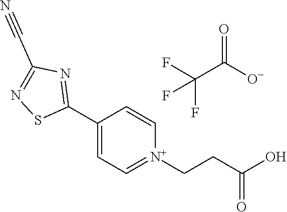 [SMILES: N#Cc1nsc(-c2cc[n+](CCC(=O)O)cc2)n1 ; O=C([O-])C(F)(F)F] | (400 MHz, D₂O) 9.17 (d, 2H), 8.63 (d, 2H), 4.95 (t, 2H), 3.21 (t, 2H) (CO₂H proton missing) |
| A90 | 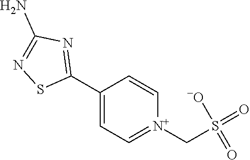 | (400 MHz, D₂O) 9.05 (d, 2H), 8.52 (d, 2H), 5.70 (s, 2H) (NH protons missing) |
| A91 | 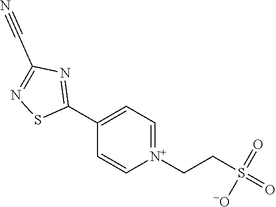 | (400 MHz, D₂O) 9.20 (d, 2H), 8.69 (d, 2H), 5.12 (t, 2H), 3.66 (t, 2H) |
| A92 | 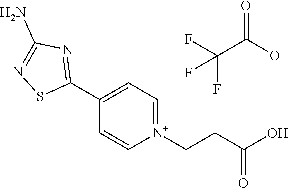 [SMILES: Nc1nsc(-c2cc[n+](CCC(=O)O)cc2)n1 ; O=C([O-])C(F)(F)F] | (400 MHz, D₂O) 9.04 (d, 2H), 8.44 (d, 2H), 4.89 (t, 2H), 3.16 (t, 2H) (CO₂H and NH protons missing) |
| A93 | 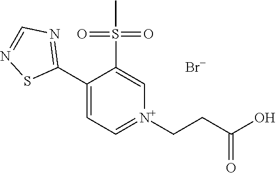 [SMILES: CS(=O)(=O)c1c[n+](CCC(=O)O)ccc1-c1ncns1 ; [Br-]] | (400 MHz, D₂O) 9.85 (s, 1H), 9.50 (d, 1H), 9.07 (s, 1H), 8.60 (d, 1H), 5.13 (t, 2H), 3.67 (s, 3H), 3.30 (t, 2H) (CO₂H proton missing) |
| A94 | 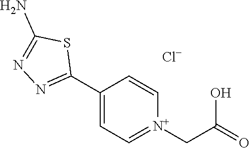 [SMILES: Nc1nnc(-c2cc[n+](CC(=O)O)cc2)s1 ; [Cl-]] | (400 MHz, D₂O) 8.88 (d, 2H), 8.36 (d, 2H), 5.49 (s, 2H) (CO₂H and NH protons missing) |
| A95 | 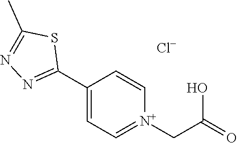 [SMILES: Cc1nnc(-c2cc[n+](CC(=O)O)cc2)s1 ; [Cl-]] | (400 MHz, D₂O) 8.92 (d, 2H), 8.53 (d, 2H), 5.38 (s, 2H), 2.87 (s, 3H) (CO₂H proton missing) |
| A96 | 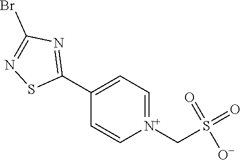 [SMILES: O=S(=O)([O-])C[n+]1ccc(-c2nc(Br)ns2)cc1] | (400 MHz, D₂O) 9.16 (d, 2H), 8.66 (d, 2H), 5.77 (s, 2H) |
| A97 | 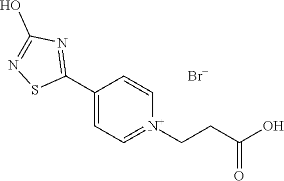 [SMILES: O=C(O)CC[n+]1ccc(-c2nc(O)ns2)cc1 ; [Br-]] | (400 MHz, D₂O) 9.07 (d, 2H), 8.45 (d, 2H), 4.90 (t, 2H), 3.18 (t, 2H) (CO₂H and OH protons missing) |
| A98 | 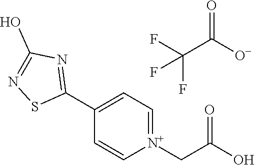 [SMILES: O=C(O)C[n+]1ccc(-c2nc(O)ns2)cc1 ; O=C([O-])C(F)(F)F] | (400 MHz, D₂O) 8.90 (d, 2H), 8.46 (d, 2H), 5.33 (s, 2H) (CO₂H and OH protons missing) |
| A99 | 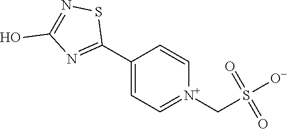 [SMILES: O=S(=O)([O-])C[n+]1ccc(-c2nc(O)ns2)cc1] | (400 MHz, D₂O) 9.08 (d, 2H) 8.56 (d, 2H) 5.72 (s, 2H) (OH proton missing) |
| A100 | 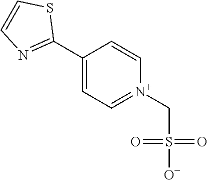 [SMILES: O=S(=O)([O-])C[n+]1ccc(-c2nccs2)cc1] | (400 MHz, D₂O) 9.02 (d, 2H), 8.62 (d, 2H), 8.25 (d, 1H), 8.13 (d, 1H), 5.73 (s, 2H) |
| A101 | 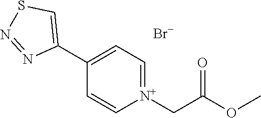 [SMILES: COC(=O)C[n+]1ccc(-c2csnn2)cc1 ; [Br-]] | (400 MHz, D₂O) 9.84 (s, 1H), 8.87 (d, 2H), 8.67 (d, 2H), 5.55 (s, 2H), 3.79 (s, 3H) |

### BIOLOGICAL EXAMPLES

### Post-Emergence Efficacy

### Method A

Seeds of a variety of test species were sown in standard soil in pots. After cultivation for 14 days (post-emergence) under controlled conditions in a glasshouse (at 24/16° C., day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted to required concentration using 0.25% or 1% Empicol ESC70 (Sodium lauryl ether sulphate)+1% ammonium sulphate as diluent. The test plants were then grown in a glasshouse under controlled conditions (at 24/16° C., day/night; 14 hours light; 65% humidity) and watered twice daily. After 13 days the test was evaluated (100=total damage to plant; 0=no damage to plant).

### Method B

Seeds of a variety of test species were sown in standard loam based soil in pots. Plants were cultivated from between 21 and 28 days (post-emergence) under controlled conditions in a glasshouse (at 24/16° C., day/night; 14 hours light; 65% humidity) for warm climate species and (at 20/16° C. day/night; 15 hours light; 65% humidity) for cool climate species.

The plants were sprayed with an aqueous spray solution derived from dissolving the technical active ingredient formula in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted to required concentration using 0.5% or 1% Empicol ESC70 (Sodium lauryl ether sulphate)+1% ammonium sulphate as diluent.

The delivery of the aqueous spray solution was via a laboratory track sprayer which delivered the aqueous spray composition at a rate of 200 litres per hectare, using a flat fan nozzle (Teejet 11002VS) and an application volume of 200 litre/ha (at 2 bar).

The test plants were then grown in a glasshouse under controlled conditions (at 24/16° C., day/night; 14 hours light; 65% humidity) for warm climate species and (at 20/16° C. day/night; 15 hours light; 65% humidity) for cool climate species and watered twice daily. After 7 and 14 days the test was evaluated (100=total damage to plant; 0=no damage to plant).

The results from method A are shown in Table B (below). A value of n/a indicates that this combination of weed and test compound was not tested/assessed.

### Test Plants for Method A:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaria faberi (SETFA)

### Test Plants for Method B:

Brassica napus (BRSNN), Solanum turberosum (SOLTU), Glycine Max (GLXMA), and Helianthus annus (HELAN)

TABLE B
Control of weed species by compounds of formula (I) after post-emergence application

| Compound | Application | | | | | | | | | |
|---|---|---|---|---|---|---|---|---|---|---|
| Number | Rate g/Ha | AMAPA | CHEAL | EPHHL | IPOHE | ELEIN | LOLPE | DIGSA | SETFA | ECHCG |
| A1 | 500 | 100 | 90 | n/a | 40 | 90 | 70 | 70 | 90 | 60 |
| A2 | 500 | 100 | 90 | n/a | 70 | 80 | 60 | 90 | 70 | 60 |
| A3 | 500 | 100 | 90 | 80 | 80 | 50 | 20 | 70 | 80 | 40 |
| A4 | 500 | 100 | 100 | 100 | 100 | 80 | 80 | 70 | 80 | 70 |
| A5 | 500 | 100 | 90 | 100 | 70 | 80 | 70 | 70 | 60 | 40 |
| A6 | 500 | 100 | 100 | 100 | 80 | 90 | 30 | 70 | 80 | 90 |
| A7 | 500 | 100 | 90 | 80 | 30 | 70 | 30 | 90 | 70 | 50 |
| A8 | 500 | n/a | 70 | 60 | 10 | 30 | 0 | 50 | 30 | 20 |
| A9 | 500 | 30 | 0 | 10 | 20 | n/a | 0 | n/a | 0 | n/a |
| A10 | 500 | 90 | 90 | n/a | 50 | 90 | 40 | 90 | 100 | 90 |
| A11 | 500 | 100 | 90 | n/a | 80 | 90 | 60 | 80 | 60 | 100 |
| A12 | 1000 | 0 | 0 | 20 | 0 | 20 | 10 | 20 | 0 | 0 |
| A13 | 500 | 100 | 50 | 70 | 30 | 50 | 10 | 60 | 70 | 50 |
| A14 | 500 | 100 | 70 | 30 | 20 | 40 | 10 | 50 | 60 | 50 |
| A15 | 500 | 0 | 0 | n/a | 20 | 10 | 0 | 30 | 30 | 30 |
| A16 | 500 | 0 | 0 | 20 | 20 | 10 | 0 | 40 | 30 | 20 |
| A17 | 500 | 70 | 70 | 50 | 20 | 10 | 0 | 40 | 40 | 30 |
| A18 | 500 | 100 | 50 | 50 | 20 | 30 | 10 | 30 | 40 | 50 |
| A19 | 500 | 100 | 50 | 50 | 50 | 30 | 30 | 30 | 40 | 40 |
| A21 | 500 | 70 | 0 | n/a | 10 | 80 | 20 | 70 | 70 | 90 |
| A22 | 500 | 10 | 10 | 40 | 0 | n/a | 0 | n/a | 0 | n/a |
| A23 | 500 | 0 | 20 | n/a | 10 | 0 | 0 | 20 | 0 | 0 |
| A24 | 500 | 0 | 40 | 40 | 20 | 0 | 0 | 10 | 0 | 0 |
| A25 | 500 | 0 | 10 | 0 | 10 | 10 | 0 | 10 | 0 | 0 |
| A26 | 500 | 0 | 10 | 20 | 10 | 10 | 10 | 20 | 10 | 10 |
| A27 | 500 | 10 | 10 | 20 | 10 | 0 | 0 | 0 | 0 | 0 |
| A28 | 500 | 100 | 80 | 20 | 70 | n/a | 30 | n/a | 80 | n/a |
| A29 | 500 | 90 | 30 | n/a | 90 | 50 | 40 | 90 | 50 | 90 |
| A30 | 500 | 90 | 70 | n/a | 50 | 90 | 30 | 90 | 90 | 90 |
| A31 | 500 | 70 | 70 | n/a | 30 | 80 | 20 | 80 | 40 | 70 |
| A32 | 500 | 60 | 20 | n/a | 0 | 40 | 10 | 50 | 20 | 70 |
| A33 | 500 | 90 | 40 | 40 | 0 | 20 | 0 | 20 | 0 | 0 |
| A34 | 500 | 70 | 0 | 0 | 0 | 0 | 0 | 0 | 0 | 0 |
| A35 | 500 | 70 | 60 | 50 | 40 | 20 | n/a | 20 | 30 | 40 |
| A36 | 500 | 70 | 60 | 60 | 30 | 10 | 0 | 30 | 10 | 10 |
| A38 | 500 | 90 | 60 | n/a | 60 | 80 | 70 | 50 | 40 | 60 |
| A39 | 500 | 40 | 30 | n/a | 20 | 0 | 0 | 0 | 0 | 0 |
| A40 | 500 | 10 | 0 | n/a | 20 | 0 | 0 | 0 | 0 | 0 |
| A41 | 500 | 10 | 0 | n/a | 0 | 0 | 0 | 10 | 10 | 10 |
| A42 | 500 | 90 | 20 | n/a | 10 | 0 | 0 | 10 | 10 | 10 |
| A43 | 500 | 40 | 40 | n/a | 30 | 60 | 10 | 30 | 20 | 60 |
| A44 | 500 | 30 | 10 | n/a | 0 | 10 | 10 | 20 | 0 | 10 |
| A45 | 500 | 0 | 0 | n/a | 0 | 50 | 0 | 20 | 0 | 10 |
| A46 | 500 | 30 | 0 | 0 | 0 | 0 | 0 | 0 | 0 | 0 |
| A47 | 500 | 60 | 60 | 10 | 10 | 10 | 0 | 10 | 10 | 10 |
| A48 | 125 | 90 | 10 | 30 | 0 | 0 | 10 | 0 | 20 | 0 |
| A49 | 500 | 10 | 50 | n/a | 10 | 20 | 20 | 50 | 20 | 10 |
| A50 | 500 | 40 | 70 | n/a | 30 | 40 | 50 | 90 | 80 | 20 |
| A51 | 500 | 10 | 60 | n/a | 10 | 10 | 20 | 50 | 30 | 30 |
| A52 | 500 | 20 | 30 | n/a | 10 | 10 | 10 | 20 | 10 | 10 |
| A53 | 500 | 10 | 10 | 20 | 20 | 0 | 0 | 0 | 10 | 10 |
| A54 | 500 | 20 | 20 | n/a | 20 | 20 | 0 | 10 | 0 | 0 |
| A55 | 500 | 50 | 40 | 10 | 30 | n/a | 30 | n/a | 20 | n/a |
| A56 | 500 | 60 | 30 | 100 | 30 | n/a | 10 | n/a | 30 | n/a |
| A57 | 500 | 100 | 80 | 20 | 0 | 20 | 0 | 30 | 0 | 60 |
| A64 | 500 | 50 | 30 | 30 | 20 | 20 | 10 | 30 | 20 | 50 |
| A65 | 500 | 30 | 20 | 20 | 20 | 20 | 10 | 30 | 10 | 20 |
| A69 | 500 | 20 | 30 | 20 | 20 | 30 | 10 | 10 | 10 | 20 |
| A71 | 500 | 20 | 50 | 40 | 30 | 20 | 0 | 30 | 10 | 20 |
| A74 | 500 | 10 | 40 | 20 | 10 | 20 | 10 | 30 | 20 | 20 |
| A79 | 500 | 90 | 40 | 60 | 40 | 90 | 30 | 80 | 70 | 80 |
| A81 | 500 | 70 | 40 | 20 | 0 | 40 | 0 | 10 | 10 | 10 |
| A83 | 500 | 20 | 30 | 50 | 10 | 10 | 10 | 20 | 10 | 0 |
| A84 | 500 | 70 | 60 | 50 | 10 | 50 | 10 | 10 | 10 | 30 |
| A85 | 500 | 100 | 90 | 100 | 100 | 100 | 90 | 100 | 100 | 100 |
| A86 | 500 | 40 | 10 | 40 | 10 | 20 | 0 | 10 | 10 | 20 |
| A87 | 500 | 0 | 10 | 20 | 0 | 0 | 0 | 0 | 20 | 20 |
| A88 | 500 | 10 | 20 | 90 | 0 | 0 | 0 | 0 | 0 | 0 |
| A89 | 500 | 30 | 30 | 20 | 10 | 80 | 10 | 90 | 80 | 60 |
| A90 | 500 | 100 | 80 | 90 | 50 | 90 | 60 | 90 | 60 | 50 |
| A92 | 500 | 100 | 60 | 80 | 20 | 20 | 10 | 80 | 60 | 60 |
| A93 | 500 | 60 | 30 | 30 | 30 | 30 | 10 | n/a | 30 | 40 |
| A94 | 500 | 30 | 40 | 30 | 10 | 40 | 10 | 30 | 10 | 20 |
| A95 | 500 | 40 | 30 | 10 | 10 | 60 | 0 | 10 | 10 | 20 |
| A96 | 500 | 100 | 100 | 100 | 30 | 90 | 100 | 100 | 80 | 100 |
| A97 | 500 | 0 | 50 | 10 | 10 | 10 | 10 | 0 | 10 | 0 |
| A98 | 500 | 50 | 40 | 30 | 10 | 10 | 0 | 70 | 20 | 40 |
| A100 | 500 | 70 | 60 | 50 | 10 | 50 | 10 | 10 | 10 | 30 |

The results from method B are shown in Table C (below). A value of n/a indicates that this combination of weed and test compound was not tested/assessed.

TABLE C
Control of weed species by compounds of formula (I) after
post-emerdence application

| Compound | Application | | | | |
|---|---|---|---|---|---|
| Number | Rate g/Ha | BRSNN | SOLTU | GLXMA | HELAN |
| A4 | 50 | 23 | 15 | 45 | |
| A4 | 150 | 32 | 30 | 83 | |
| A4 | 600 | 43 | 82 | 93 | |
| A4 | 50 | 43 | 50 | 52 | 97 |
| A4 | 150 | 57 | 60 | 62 | 100 |
| A4 | 600 | 80 | 67 | 85 | 100 |

1. A method of controlling unwanted plant growth, comprising applying a compound of formula (I): 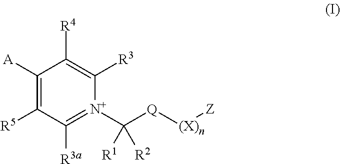
wherein
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O; and
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR¹⁵a —NH₂—NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³, R^{3a}, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
A is a 5-membered heteroaryl attached to the rest of the molecule via a ring carbon atom, which comprises 1, 2, 3 or 4 heteroatoms independently selected from the group consisting of N, O and S, and wherein the heteroaryl may, where feasible, be optionally substituted by 1, 2 or 3 R⁸ substituents, which may be the same or different, and wherein when A is substituted on one or more ring carbon atoms, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;
and/or
when A is substituted on a ring nitrogen atom, R⁸ is selected from the group consisting of —OR⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy and C₃-C₆alkynyloxy; and
each R⁹ is independently selected from the group consisting of halogen, cyano, —OH, —N(R⁶)₂, C₁-C₄alkyl, C₁-C₄alkoxy, C₁-C₄haloalkyl and C₁-C₄haloalkoxy;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;
n is 0 or 1;
Z is selected from the group consisting of —C(O)OR¹⁰, —CH₂OH, —CHO, —C(O)NHOR¹¹, —C(O)NHCN, —OC(O)NHOR¹¹, —OC(O)NHCN, —NR⁶C(O)NHOR¹¹, —NR⁶C(O)NHCN, —C(O)NHS(O)₂R¹², —OC(O)NHS(O)₂R¹², —NR⁶C(O)NHS(O)₂R¹², —S(O)₂OR¹⁰, —OS(O)₂OR¹⁰, —NR⁶S(O)₂OR¹⁰, —NR⁶S(O)OR¹⁰, —NHS(O)₂R¹⁴, —S(O)OR¹⁰, —OS(O)OR¹⁰, —S(O)₂NHCN, —S(O)₂NHC(O)R¹⁸, —S(O)₂NHS(O)₂R¹², —OS(O)₂NHCN, —OS(O)₂NHS(O)₂R¹², —OS(O)₂NHC(O)R¹⁸, —NR⁶S(O)₂NHCN, —NR⁶S(O)₂NHC(O)R¹⁸, —N(OH)C(O)R¹⁵, —ONHC(O)R¹⁵, —NR⁶S(O)₂NHS(O)₂R¹², —P(O)(R¹³)(OR¹⁰), —P(O)H(OR¹⁰), —OP(O)(R¹³)(OR¹⁰), —NR⁶P(O)(R¹³)(OR¹⁰) and tetrazole;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹¹ is selected from the group consisting of hydrogen, C₁-C₆alkyl and phenyl, and wherein said phenyl is optionally substituted by 1, 2 or 3 R⁹ substituents, which may be the same or different;
R¹² is selected from the group consisting of C₁-C₆alkyl, C₁-C₆haloalkyl, C₁-C₆alkoxy, —OH, —N(R⁶)₂ and phenyl, and wherein said phenyl is optionally substituted by 1, 2 or 3 R⁹ substituents, which may be the same or different;
R¹³ is selected from the group consisting of —OH, C₁-C₆alkyl, C₁-C₆alkoxy and phenyl;
R¹⁴ is C₁-C₆haloalkyl;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
R¹⁸ is selected from the group consisting of hydrogen, C₁-C₆alkyl, C₁-C₆haloalkyl, C₁-C₆alkoxy, —N(R⁶)₂ and phenyl, and wherein said phenyl is optionally substituted by 1, 2 or 3 R⁹ substituents, which may be the same or different;
and
r is 0, 1 or 2,
to the unwanted plants or to the locus thereof.

2. A compound of formula (I) or an agronomically acceptable salt or zwitterionic species thereof, as defined in claim 1, with the proviso that:
i) in the compound of formula (I), A is not selected from the group consisting of formula A-Ib to A-IIIb below 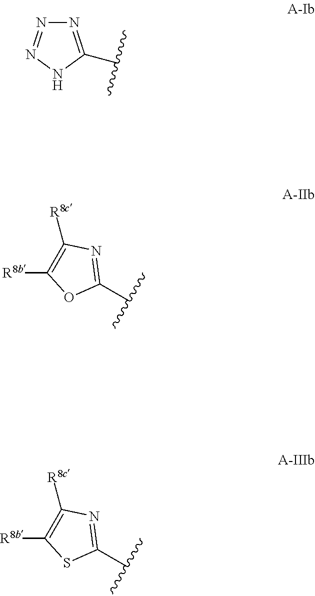
wherein each R^{8b′} is independently selected from the group consisting of phenyl, 4-methoxyphenyl, 4-butoxyphenyl, 4-fluorophenyl and methoxy, and each R^{8c′}, is independently hydrogen or methyl;
or
ii) the compound of formula (I) is not selected from the group consisting of:

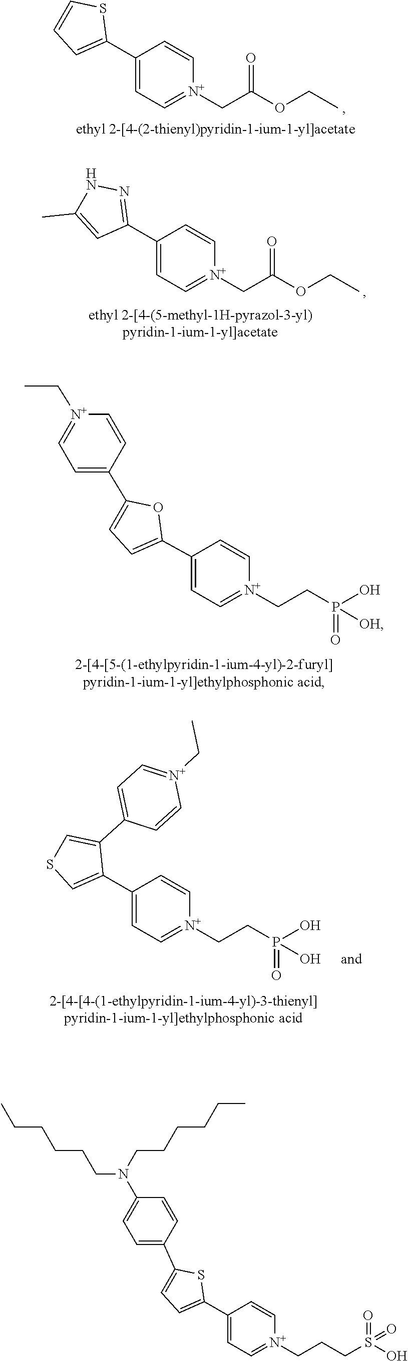

3. The compound of formula (I) according to claim 2, wherein R¹ and R² are independently selected from the group consisting of hydrogen and C₁-C₆alkyl.

4. The compound of formula (I) according to claim 2, wherein R¹ and R² are hydrogen.

5. The compound of formula (I) according to claim 2, wherein each R^{1a} and R^{2b} are independently selected from the group consisting of hydrogen, C₁-C₆alkyl, —OH and —NH₂.

6. The compound of formula (I) according to claim 2, wherein each R^{1a} and R^{2b} are hydrogen.

7. The compound of formula (I) according to claim 2, wherein m is 0, 1 or 2.

8. The compound of formula (I) according to claim 2, wherein R³, R^{3a}, R⁴ and R⁵ are independently selected from the group consisting of hydrogen, halogen, cyano, C₁-C₆alkyl and C₁-C₆fluoroalkyl.

9. The compound of formula (I) according to claim 2, wherein R³, R^{3a}, R⁴ and R⁵ are hydrogen.

10. The compound of formula (I) according to claim 2, wherein A is selected from the group consisting of formula A-I to A-XXXII below 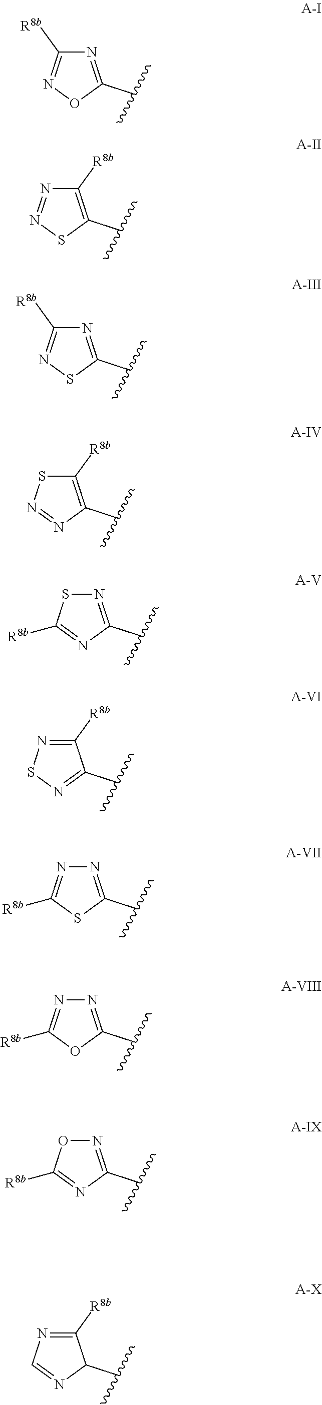 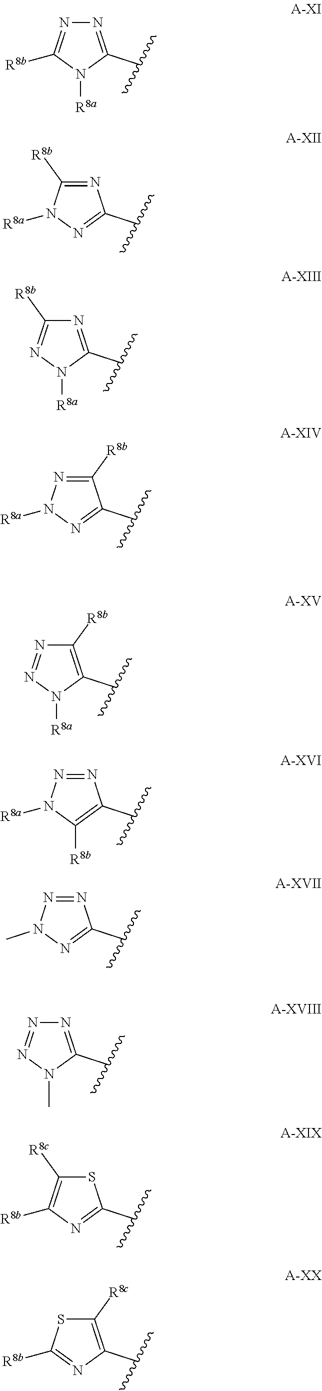 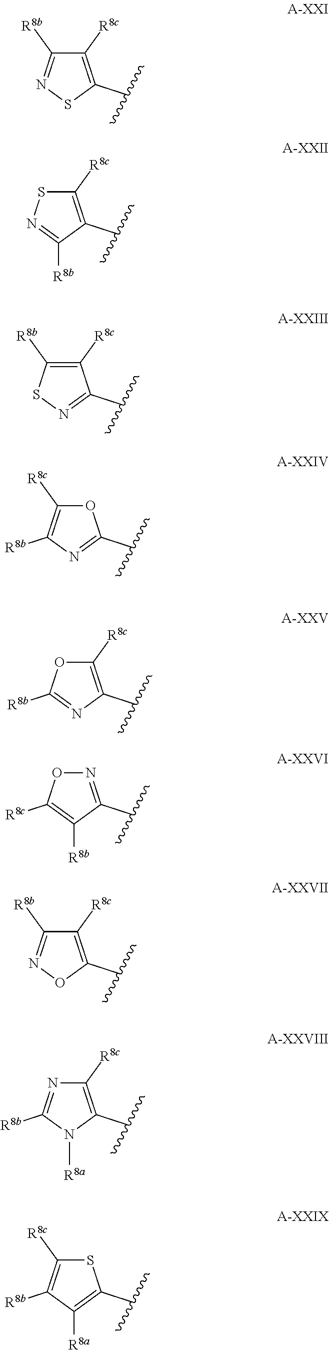 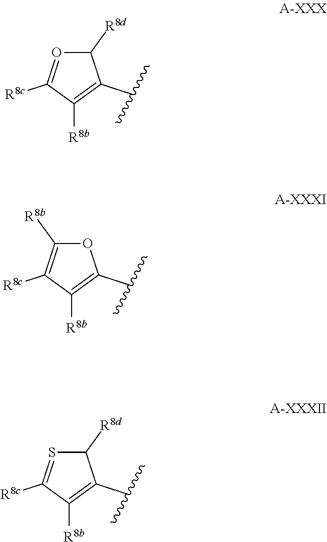
wherein the jagged line defines the point of attachment to a compound of formula (I);
R^{8a} is selected from the group consisting of hydrogen, C₁-C₆alkyl and C₁-C₆haloalkyl;
each R^{8b}, R^{8c} and R^{8d} are independently selected from the group consisting of hydrogen, halogen, nitro, cyano, —NH₂, —S(O)ᵣR¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl;
and R¹⁰, R¹⁵, R¹⁶, R¹⁷ and r are as defined in claim 1.

11. The compound of formula (I) according to claim 2, wherein A is selected from the group consisting of formula A-I to A-III below 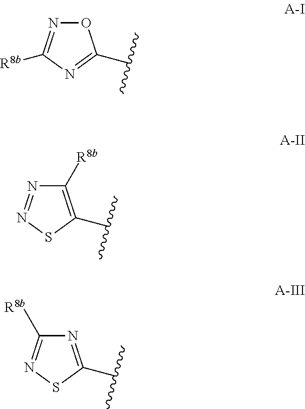
wherein the jagged line defines the point of attachment to a compound of formula (I);
each R^{8b} is independently selected from the group consisting of hydrogen, halogen, cyano, —NH₂, —C(O)NR¹⁶R¹⁷, C₁-C₆alkyl and C₁-C₆haloalkyl;
and R¹⁶ and R¹⁷ are as defined in claim 1.

12. The compound of formula (I) according to claim 2, wherein A is selected from the group consisting of formula A-Ia to A-Xa below

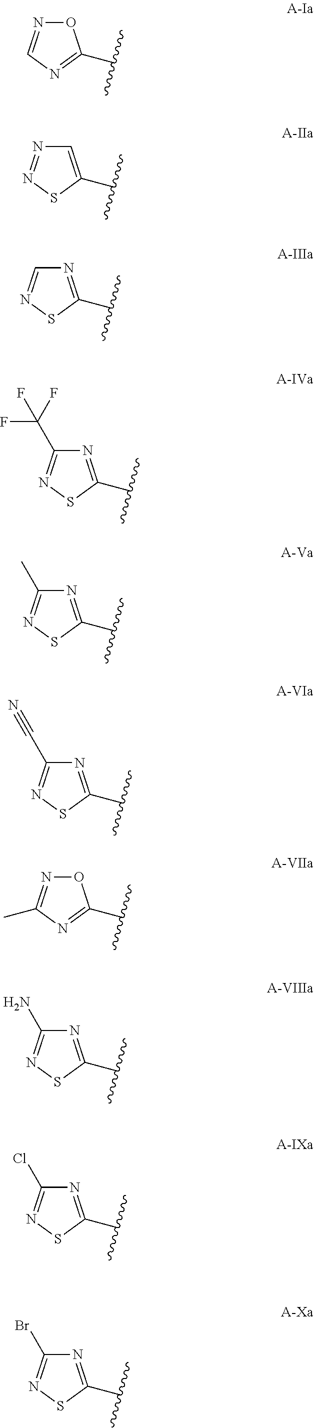

13. The compound of formula (I) according to claim 2, wherein Z is selected from the group consisting of —C(O)OR¹⁰, —C(O)NHS(O)₂R¹², —OS(O)₂OR¹⁰, —S(O)₂OR¹⁰, and —P(O)(R¹³)(OR¹⁰).

14. The compound according to claim 2, wherein Z is —C(O)OH or —S(O)₂OH.

15. The compound according to claim 2, wherein n is 0.

16. (canceled)

17. The method of claim 1, wherein the applying is pre-harvest desiccation in crops.

18. An agrochemical composition comprising a herbicidally effective amount of a compound of formula (I) as defined in claim 1 and an agrochemically-acceptable diluent or carrier.

19. A method of controlling unwanted plant growth, comprising applying a compound of formula (I) as defined in claim 2, to the unwanted plants or to the locus thereof.
